(19) United States
(12) Patent Application Publication
(10) Pub. No.: US 2026/0325673 A1
(43) Pub. Date: Oct. 22, 2026

(54) LOCO-REGIONAL PERFUSION OF A KIDNEY FOR LOCALIZED GENE THERAPY

(71) Applicant: DINAQOR AG, Schlieren (CH)

(72) Inventors: Josef EL ANDARI, Zürich (CH); Mark DEHDASHTIAN, Irvine, CA (US); Éduard AYUSO LÓPEZ, Zürich (CH); Goutham Kumar GANJAM, Oberengstringen (CH); Christopher P. RUSCONI, Durham, NC (US)

(21) Appl. No.: 19/117,544

(22) PCT Filed: Oct. 2, 2023
(86) PCT No.: PCT/EP2023/077263

§ 371 Date: Apr. 1, 2025

Related U.S. Application Data
(60) Provisional application No. 63/412,796, filed on Oct. 3, 2022.

(51) Int. Cl. A61K 48/00; A61K 38/17; A61M 1/16; A61M 1/36; A61M 25/04; A61M 25/10; C12N 15/86

(52) U.S. Cl. CPC A61K 48/0033; A61K 38/177; A61K 48/0058; A61K 48/0075; A61M 1/16; A61M 1/3659; A61M 25/1011; C12N 15/86; A61M 25/04; A61M 2025/1052; C12N 2750/14143; C12N 2830/008

(57) ABSTRACT

Disclosed is a method for treating a renal condition by loco-regional perfusion of one or both of a patient's kidneys. A closed circuit may be formed with a perfusion catheter positioned in the renal artery of the kidney, a recovery catheter positioned in the renal vein of the kidney, and an external membrane oxygenator disposed therebetween. A perfusate containing, for example, a gene therapy drug may be circulated through the closed circuit while isolating the closed circuit from the patient's systemic circulation.

### CROSS-REFERENCE TO RELATED APPLICATION(S)

This application claims the benefit of priority of U.S. Provisional Patent Application No. 63/412,796, filed Oct. 3, 2022, the disclosure of which is hereby incorporated by reference herein in its entirety.

### TECHNICAL FIELD

The present invention relates to treatment of renal diseases, and, in particular, to localized delivery of therapeutic agents to a patient's kidney.

### BACKGROUND

Gene therapy and cell therapy techniques in the treatment of various renal conditions, such as chronic kidney disease, have attracted increased attention due to their potential to be uniquely tailored and efficacious in addressing the root cause pathogenesis of various renal conditions. Nevertheless, issues related to delivery, including vector efficiency, dose, specificity, and safety remain. As such, there is a need for further research directed to ways of achieving a more targeted, homogenous delivery to the kidney of drugs suitable for treatment of various renal conditions that are also effective, well tolerated, and minimally invasive.

### SUMMARY

The following presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect, a gene therapy vector adapted for transduction of renal cells of a human subject comprises: an adeno-associated virus (AAV) vector; and a polynucleotide sequence packaged in the AAV vector, the polynucleotide sequence encoding a therapeutic protein having at least 80% sequence identity to a sequence selected from the group consisting of SEQ ID NO: 3, SEQ ID NO: 5, and SEQ ID NO: 7.

In at least one embodiment, the polynucleotide sequence encodes for a therapeutic protein having at least 80% sequence identity to SEQ ID NO: 3.

In at least one embodiment, the polynucleotide sequence encodes for a therapeutic protein having at least 80% sequence identity to SEQ ID NO: 5.

In at least one embodiment, the polynucleotide sequence encodes for a therapeutic protein having at least 80% sequence identity to SEQ ID NO: 7.

In at least one embodiment, the polynucleotide sequence further comprises a promoter sequence operatively linked to the polynucleotide sequence encoding for the therapeutic protein.

In at least one embodiment, the promoter sequence is selected from the group consisting of: SEQ ID NO: 9, SEQ ID NO: 10, SEQ ID NO: 11, SEQ ID NO: 12, SEQ ID NO: 13, and SEQ ID NO: 14.

In at least one embodiment, the promoter sequence is selected from the group consisting of: SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28.

In at least one embodiment, a serotype of the AAV vector is selected from the group consisting of AAV1, AAV2, AAV3, AAV4, AAV5, AAV6, AAV7, AAV8, AAV9, AAV10, AAV11, AAV12, and AAV13. In at least one embodiment, a serotype of the AAV vector is AAV5.

In another aspect, a gene therapy drug comprises: the gene therapy vector of any of the preceding embodiments; and a pharmaceutically acceptable carrier.

In another aspect, method of treating a kidney-related disease comprises administering to a patient in need thereof a therapeutic dose of the gene therapy drug of the preceding embodiment.

In another aspect, a method of performing gene replacement of a mutated gene comprises administering to a patient in need thereof a therapeutic dose of the gene therapy drug of a preceding embodiment.

In another aspect, a method of treating autosomal dominant polycystic kidney disease (ADPKD) in a subject comprises: administering to the subject a therapeutic dose of a drug comprising the gene therapy vector of any of the preceding embodiments and a pharmaceutically acceptable carrier.

In another aspect, a method of performing localized delivery of a polynucleotide sequence to renal cells in a kidney of a mammalian subject comprises: positioning a perfusion catheter in the renal artery of the kidney; positioning a recovery catheter in the renal vein of the kidney, wherein the perfusion catheter and the recovery catheter together with a membrane oxygenation device form a closed perfusion circuit through the kidney; and causing a perfusate to flow through the closed circuit, wherein the perfusate comprises the gene therapy drug of a preceding embodiment, and wherein the closed circuit substantially isolates perfusion through the kidney from the systemic circulation of the subject.

In at least one embodiment, the renal cells comprise tubular cells.

In at least one embodiment, a dose of the AAV vector is delivered via the closed circuit and maintained at a concentration of at least about 5×10⁹ of vector genome per milliliter (mL) of plasma) during perfusion, and wherein the vector present leaking into systemic circulation of the subject remains less than 5×10⁷ of vector genome per mL of plasma during perfusion, wherein the perfusion is maintained for a total of about 30 minutes to about 90 minutes.

In at least one embodiment, positioning the perfusion catheter in the renal artery comprises positioning the perfusion catheter via the arteria femoralis.

In at least one embodiment, positioning the recovery catheter in the renal vein comprises positioning the perfusion catheter via percutaneous access through the vena femoralis or via the jugular vein.

In at least one embodiment, positioning the recovery catheter in the renal vein comprises positioning the perfusion catheter via non-percutaneous cut-down access.

In at least one embodiment, causing the perfusate to flow through the closed circuit comprises: causing the perfusate to pass through the membrane oxygenation device prior to entering the renal artery via the perfusion catheter.

In at least one embodiment, the method further comprises: adding additional perfusate to the closed circuit or diluting the perfusate by about 5% to about 50% v/v of a saline solution to account for bladder excretion volume.

In at least one embodiment, the closed circuit maintains a flow rate of the perfusate at about 500 mL/min/1.73 m² of body surface area per kidney to about 650 mL/min/1.73 m² of body surface area per kidney for about 15 min to about 4 hours.

In at least one embodiment, the closed circuit maintains a flow rate of the perfusate at about 150 mL/min/1.73 m² of body surface area per kidney to about 700 mL/min/1.73 m² of body surface area per kidney for about 15 min to about 4 hours.

In at least one embodiment, the method further comprises: applying negative pressure at the recovery catheter, wherein the negative pressure ranges from about −100 mmHg to 120 mmHg.

In at least one embodiment, one or more of the perfusion catheter and the recovery catheter are introduced percutaneously or non-percutaneously.

In at least one embodiment, less than about 20% v/v, less than about 15% v/v, less than about 10% v/v, less than about 5% v/v, less than about 4% v/v, less than about 3% v/v, less than about 2% v/v, less than about 1% v/v, less than about 0.5% v/v, or substantially no (0% v/v) perfusate circulated through the closed circuit leaks outside of the closed circuit.

In at least one embodiment, one or more of the perfusion catheter or the recovery catheter is a balloon catheter.

In another aspect, a method of delivering a therapeutic composition to a subject in need thereof, comprises locally delivering the therapeutic composition to a kidney of the subject while substantially avoiding introduction of the therapeutic composition into the systemic circulation or other organs, the therapeutic composition comprising the gene therapy drug of a preceding embodiment.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 illustrates a schematic of a first exemplary recovery catheter having a single balloon structure in accordance with at least one embodiment;

FIG. 2 is a photograph of a recovery catheter produced according to an embodiment of the first exemplary recovery catheter;

FIG. 3 illustrates deployment of the first exemplary recovery catheter in accordance with at least one embodiment;

FIG. 4 illustrates deployment of a second exemplary recovery catheter having a single balloon structure in accordance with at least one embodiment;

FIG. 5 illustrates deployment of a third exemplary recovery catheter and a fourth exemplary recovery catheter each having a single balloon structure in accordance with at least one embodiment;

FIG. 6 illustrates deployment of a fifth exemplary recovery catheter having a single balloon structure and a sixth exemplary recovery catheter without a balloon structure in accordance with at least one embodiment;

FIG. 7 illustrates deployment of a seventh exemplary recovery catheter having multiple balloon structures in accordance with at least one embodiment;

FIG. 8 illustrates deployment of an eighth exemplary recovery catheter having a partially covered and recapturable stent structure in accordance with at least one embodiment;

FIG. 9 illustrates deployment of a ninth exemplary recovery catheter having a deployable and retractable stent structure and a balloon structure in accordance with at least one embodiment;

FIG. 10 illustrates deployment of a tenth exemplary recovery catheter having a covered disk-shaped stent structure in accordance with at least one embodiment;

FIG. 11A is a schematic of a first exemplary perfusion catheter having a single balloon structure in accordance with at least one embodiment;

FIG. 11B is a schematic of the balloon structure of the first exemplary perfusion catheter in an expanded state in accordance with at least one embodiment;

FIG. 11C is a schematic of the balloon structure of the first exemplary perfusion catheter in a retracted state in accordance with at least one embodiment;

FIG. 12A is a schematic of a second exemplary perfusion catheter having distal plug in accordance with at least one embodiment;

FIG. 12B is a schematic of the plug of the second exemplary perfusion catheter in accordance with at least one embodiment;

FIG. 12C is a schematic of the plug of the second exemplary perfusion catheter in an extended state in accordance with at least one embodiment;

FIG. 13A is a schematic of a third exemplary perfusion catheter having a distal wedge in accordance with at least one embodiment;

FIG. 13B is a schematic of the wedge of the third exemplary perfusion catheter in accordance with at least one embodiment;

FIG. 13C is a further schematic of the distal end of the third exemplary perfusion catheter in an extended state in accordance with at least one embodiment;

FIG. 14A illustrates deployment of a fourth exemplary perfusion catheter having a partially covered and recapturable stent structure in accordance with at least one embodiment;

FIG. 14B illustrates the stent structure of the fourth exemplary perfusion catheter in a retracted state in accordance with at least one embodiment;

FIG. 14C illustrates the stent structure of the fourth exemplary perfusion catheter in a deployed state in accordance with at least one embodiment;

FIG. 15A illustrates deployment of a fifth exemplary perfusion catheter having a releasable covered braided disk in accordance with at least one embodiment;

FIG. 15B illustrates the braided disk of the fifth exemplary perfusion catheter in a deployed state in accordance with at least one embodiment;

FIG. 16A is a schematic of a sixth exemplary perfusion catheter having a tapered lumen shaft in accordance with at least one embodiment;

FIG. 16B illustrates deployment of the sixth exemplary perfusion catheter in accordance with at least one embodiment;

FIG. 16C illustrates a pre-shaped lumen shaft of the sixth exemplary perfusion catheter in accordance with at least one embodiment;

FIG. 17 illustrates exemplary pre-formed lumen shafts for the exemplary catheters according to the various embodiments;

FIG. 18 depicts an exemplary loco-regional perfusion system in accordance with embodiments of the present disclosure;

FIG. 19 is a schematic of an exemplary loco-regional perfusion device in accordance with embodiments of the present disclosure;

FIG. 20 includes radiographs showing placement of arterial and venous catheters in the renal artery and renal vein, respectively, of a porcine kidney before (upper image) and after (lower image) venous injection of a contrast agent;

FIG. 21 is a plot showing kidney transduction and biodistribution after 60 min of kidney LRP performed in accordance with embodiments of the present disclosure;

FIG. 22A shows vector genome per mL of plasma measured at various time points during a 60-minute kidney LRP procedure with a high vector genome dose;

FIG. 22B shows vector genome per mL of plasma measured at various time points during a 45-minute kidney LRP procedure with a low vector genome dose;

FIG. 23A is a plot of C3a levels for several days post kidney LRP treatment for two different animals;

FIG. 23B is a plot of % transduction inhibition for various sample dilutions;

FIG. 24A is a plot of flow rate during kidney LRP;

FIG. 24B is a plot of pump speed during the kidney LRP; and

FIG. 25 is a plot showing vector genome per mL of plasma measured at various time points during a 60-minute kidney LRP procedure in the LRP closed circuit versus systemic circulation for a perfusate comprising multiple AAV serotypes;

FIG. 26 is a plot showing biodistribution of the multiple AAV serotypes in the kidney tissue after being perfused simultaneously for 60 minutes;

FIG. 27 is a plot showing biodistribution in the kidney compared to other the liver after perfusion for 60 minutes with the multiple AAV serotypes;

FIG. 28 is a plot showing relative quantification of transgene mRNA levels in the treated kidney sections for AAV5 versus cumulative AAV in the kidney;

FIG. 29 is a plot showing vector genome per mL of plasma measured at various time points during a 60-minute kidney LRP procedure in the LRP closed circuit versus systemic circulation for AAV5 delivery;

FIG. 30 is a plot showing biodistribution in a treated kidney compared to the liver and the untreated kidney after perfusion in the treated kidney for 60 minutes with AAV5.

FIG. 31 is a plot showing vector genome per mL of plasma measured at various time points during a 52-minute kidney LRP procedure in the LRP closed circuit versus systemic circulation for AAV5 delivery;

FIG. 32 is a plot showing biodistribution in a treated kidney compared to the liver, the untreated kidney, and other regions after perfusion in the treated kidney for 52 minutes with AAV5;

FIG. 33A is a schematic of an exemplary perfusion catheter having a balloon in a retracted state in accordance with at least one embodiment;

FIG. 33B is a schematic of the exemplary perfusion catheter having its balloon in a deployed state in accordance with at least one embodiment;

FIG. 33C is a photograph of an exemplary perfusion catheter with its balloon in the deployed state in accordance with at least one embodiment;

FIG. 34A is a schematic of an exemplary recovery catheter having a balloon in a retracted state in accordance with at least one embodiment;

FIG. 34B is a schematic of the exemplary recovery catheter having its balloon in a deployed state in accordance with at least one embodiment;

FIG. 34C is a photograph of an exemplary recovery catheter with its balloon in the deployed state in accordance with at least one embodiment;

FIG. 35A illustrates deployment of a single perfusion catheter deployed within the renal artery of the left kidney in accordance with at least one embodiment;

FIG. 35B illustrates deployment of a pair of perfusion catheters deployed within the renal artery of the left kidney in accordance with at least one embodiment;

FIG. 36A illustrates deployment of a single recovery catheter deployed within the renal vein of the left kidney in accordance with at least one embodiment;

FIG. 36B illustrates deployment of a pair of recovery catheters deployed within the renal vein of the left kidney in accordance with at least one embodiment;

FIG. 37 is a plot showing shedding analysis (vector genome per mL of plasma or urine) detected in the LRP circulation, systemic circulation, and urine of an LRP-treated animal;

FIG. 38 is a plot showing total vector genomes detected in urine of the LRP-treated animal;

FIG. 39 is a plot showing biodistribution analysis of the LRP-treated kidney divided by kidney sections compared to the untreated kidney and liver;

FIG. 40 is a plot showing GFP protein in the LRP-treated kidney compared to untreated kidney and liver;

FIG. 41 is a plot showing shedding analysis detected in the LRP circuit, systemic circulation, and urine of an LRP-treated animal;

FIG. 42 is a plot showing shedding analysis detected in the systemic circulation and urine of an animal treated via systemic administration;

FIG. 43 is a plot showing total vector genome present in the urine of the LRP-treated animal;

FIG. 44 is a plot showing total vector genome present in the urine of the animal treated via systemic administration;

FIG. 45 is a plot modeling concentration vs. time for AAV5 in the LRP circuit of the LRP-treated kidney;

FIG. 46 is plot modeling concentration vs. time for AAV5 in systemic circulation of animal treated via systemic administration;

FIG. 47 is a plot showing biodistribution analysis measured in kidney sections compared to other organs from the LRP-treated animal;

FIG. 48 is a plot showing biodistribution analysis measured in kidney sections compared to other organs from the animal treated via systemic administration;

FIG. 49 illustrates a first exemplary construct for texting gene expression in renal cells in accordance with at least one embodiment;

FIG. 50 illustrates a second exemplary construct for texting gene expression in renal cells in accordance with at least one embodiment;

FIG. 51 illustrates a third exemplary construct for texting gene expression in renal cells in accordance with at least one embodiment;

FIG. 52 illustrates a fourth exemplary construct for texting gene expression in renal cells in accordance with at least one embodiment;

FIG. 53 illustrates a fifth exemplary construct for texting gene expression in renal cells in accordance with at least one embodiment;

FIG. 54 illustrates a sixth exemplary construct for texting gene expression in renal cells in accordance with at least one embodiment; and

FIG. 55 illustrates a seventh exemplary construct for texting gene expression in renal cells in accordance with at least one embodiment.

### DEFINITIONS

As used herein, the singular forms “a,” “an,” and “the” include plural references unless the context clearly indicates otherwise. Thus, for example, reference to “a drug” includes a single drug as well as a mixture of two or more different drugs; and reference to a “viral vector” includes a single viral vector as well as a mixture of two or more different viral vectors, and the like.

Also as used herein, “about,” when used in connection with a measured quantity, refers to the normal variations in that measured quantity, as expected by one of ordinary skill in the art in making the measurement and exercising a level of care commensurate with the objective of measurement and the precision of the measuring equipment. In at least one embodiment, the term “about” includes the recited number±10%, such that “about 10” would include from 9 to 11.

Also as used herein, “polynucleotide” has its ordinary and customary meaning in the art and includes any polymeric nucleic acid such as DNA or RNA molecules, as well as chemical derivatives known to those skilled in the art. Polynucleotides include not only those encoding a therapeutic protein, but also include sequences that can be used to decrease the expression of a targeted nucleic acid sequence using techniques known in the art (e.g., antisense, interfering, or small interfering nucleic acids). Polynucleotides can also be used to initiate or increase the expression of a targeted nucleic acid sequence or the production of a targeted protein within cells of the cardiovascular system. Targeted nucleic acids and proteins include, but are not limited to, nucleic acids and proteins normally found in the targeted tissue, derivatives of such naturally occurring nucleic acids or proteins, naturally occurring nucleic acids or proteins not normally found in the targeted tissue, or synthetic nucleic acids or proteins. One or more polynucleotides can be used in combination, administered simultaneously and/or sequentially, to increase and/or decrease one or more targeted nucleic acid sequences or proteins.

Also as used herein, “perfusion,” “perfused,” and “perfusing” have their ordinary and customary meaning in the art and refer to administration for a time period (typically a minute or more) that is substantially longer than the art recognized term of “injection” or “bolus injection” (typically less than a minute). The flow rate of the perfusion will depend at least in part on the volume administered.

Also as used herein, “exogenous” nucleic acids or genes are those that do not occur in nature in the vector utilized for nucleic acid transfer; e.g., not naturally found in the viral vector, but the term is not intended to exclude nucleic acids encoding a protein or polypeptide that occurs naturally in the patient or host.

Also as used herein, “renal cell” includes any cell of a kidney that is involved in maintaining a structure or providing a function of the kidney. Examples of renal cells, without limitation, include renal tubular epithelial cells (or “tubular cells”) and podocytes.

Also as used herein, “isolated,” “substantially isolated,” “largely isolated,” and their variants are terms that do not require complete or absolute isolation of the renal or systemic circulation; rather, they are intended to mean that a majority, preferably the major part or even substantially all of the specified circulation is isolated. Also as used herein, “partially isolated” refers to any nontrivial portion of the specified circulation being isolated.

Also as used herein, “non-naturally restricted” includes any method of restricting the flow of fluid through a blood vessel, e.g., balloon catheter, sutures, etc., but does not include naturally occurring restriction, e.g., plaque build-up (stenosis). Non-natural restriction includes substantial or total isolation of, for example, the renal circulation.

Also as used herein, “minimally invasive” is intended to include any procedure that does not require open surgical access to the kidney or vessels closely associated with the kidney. Such procedures include the use of endoscopic means to access the kidney, and also catheter-based means relying on access via large arteries and veins.

Also as used herein, “adeno-associated virus” or “AAV” encompasses all subtypes, serotypes, and pseudotypes, as well as naturally occurring and recombinant forms. A variety of AAV serotypes and strains are known in the art and are publicly available from sources, such as the ATCC and academic or commercial sources. Alternatively, sequences from AAV serotypes and strains which are published and/or available from a variety of databases may be synthesized using known techniques.

Also as used herein, “serotype” refers to an AAV which is identified by and distinguished from other AAVs based on capsid protein reactivity with defined antisera. There are at least twelve known serotypes of human AAV, including AAV1 through AAV13, however additional serotypes continue to be discovered, and use of newly discovered serotypes are contemplated.

Also as used herein, “pseudotyped” AAV refers to an AAV that contains capsid proteins from one serotype and a viral genome including 5′ and 3′ inverted terminal repeats (ITRs) of a different or heterologous serotype. A pseudotyped recombinant AAV (rAAV) would be expected to have cell surface binding properties of the capsid serotype and genetic properties consistent with the ITR serotype. A pseudotyped rAAV may comprise AAV capsid proteins, including VP1, VP2, and VP3 capsid proteins, and ITRs from any serotype AAV, including any primate AAV serotype from AAV1 through AAV13, as long as the capsid protein is of a serotype heterologous to the serotype(s) of the ITRs. In a pseudotyped rAAV, the 5′ and 3′ ITRs may be identical or heterologous. Pseudotyped rAAV are produced using standard techniques described in the art.

Also as used herein, a “chimeric” rAAV vector encompasses an AAV vector comprising heterologous capsid proteins; that is, a rAAV vector may be chimeric with respect to its capsid proteins VP1, VP2, and VP3, such that VP1, VP2, and VP3 are not all of the same serotype AAV. A chimeric AAV as used herein encompasses AAV such that the capsid proteins VP1, VP2, and VP3 differ in serotypes, including for example but not limited to capsid proteins from AAV1 and AAV2; are mixtures of other parvo virus capsid proteins or comprise other virus proteins or other proteins, such as for example, proteins that target delivery of the AAV to desired cells or tissues. A chimeric rAAV as used herein also encompasses an rAAV comprising chimeric 5′ and 3′ ITRs. A chimeric rAAV as used herein may also comprise capsids generated from non-AAV sequences, such as those obtained via peptide display screening.

Also as used herein, a “pharmaceutically acceptable excipient” or “pharmaceutically acceptable carrier” refers to any inert ingredient in a composition that is combined with an active agent in a formulation. A pharmaceutically acceptable excipient/carrier can include, but is not limited to, carbohydrates (such as glucose, sucrose, or dextrans), antioxidants (such as ascorbic acid or glutathione), chelating agents, low-molecular weight proteins, high-molecular weight polymers, gel-forming agents, or other stabilizers and additives. Other examples of a pharmaceutically acceptable excipient/carrier include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

Also as used herein, a “patient” refers to a subject, particularly a human (but could also encompass a non-human), who has presented a clinical manifestation of a particular symptom or symptoms suggesting the need for treatment, who is treated prophylactically for a condition, or who has been diagnosed with a condition to be treated.

Also as used herein, a “subject” encompasses the definition of the term “patient” and does not exclude individuals who are otherwise healthy.

Also as used herein, “treatment of” and “treating” include the administration of a drug with the intent to lessen the severity of or prevent a condition, e.g., a renal condition or renal disease.

Also as used herein, “prevention of” and “preventing” include the avoidance of the onset of a condition, e.g., a renal condition or renal disease.

Also as used herein, a “condition” or “conditions” refers to those medical conditions, such as a renal disease, that can be treated, mitigated, or prevented by administration to a subject of an effective amount of a drug.

Also as used herein, an “effective amount” refers to the amount of a drug that is sufficient to produce a beneficial or desired effect at a level that is readily detectable by a method commonly used for detection of such an effect. In some embodiments, such an effect results in a change of at least 10% from the value of a basal level where the drug is not administered. In other embodiments, the change is at least 20%, 50%, 80%, or an even higher percentage from the basal level. As will be described below, the effective amount of a drug may vary from subject to subject, depending on age, general condition of the subject, the severity of the condition being treated, the particular drug administered, and the like. An appropriate “effective” amount in any individual case may be determined by one of ordinary skill in the art by reference to the pertinent texts and literature and/or by using routine experimentation.

Also as used herein, an “active agent” refers to any material that is intended to produce a therapeutic, prophylactic, or other intended effect, whether or not approved by a government agency for that purpose.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

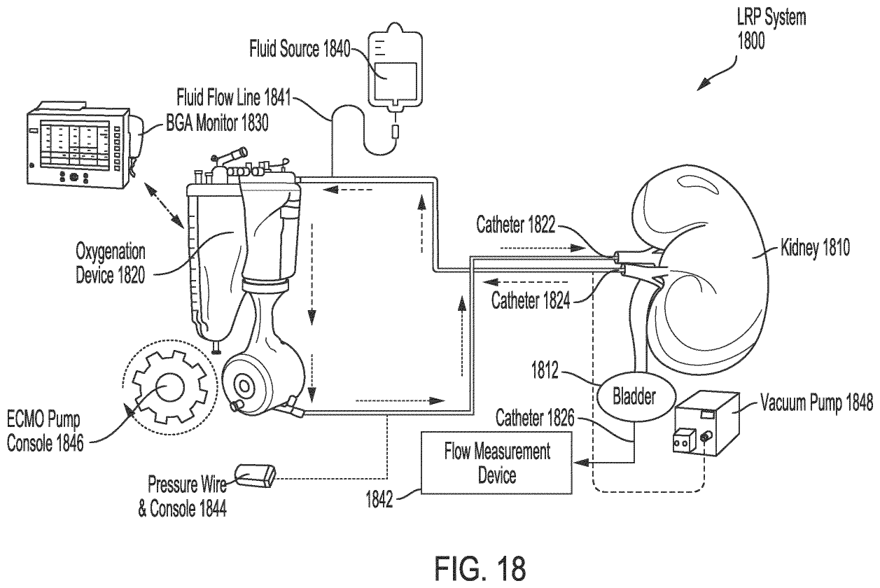

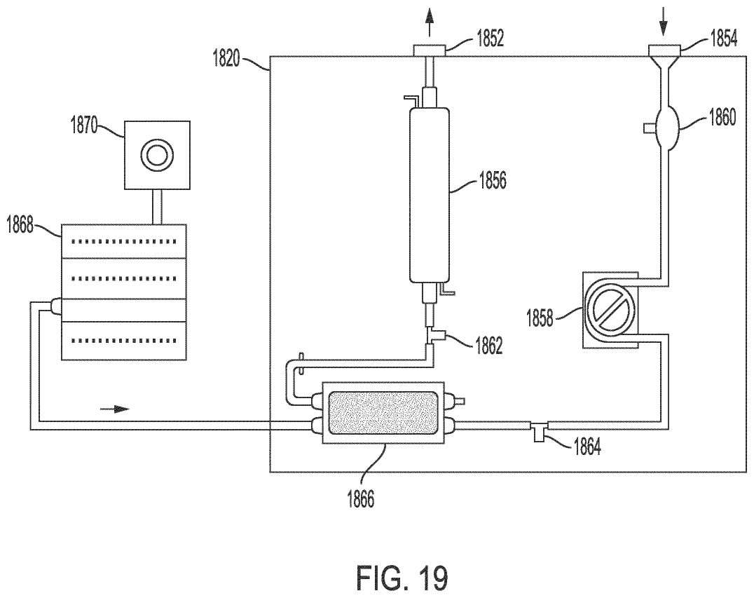

Certain embodiments of the present disclosure are directed to systems and methods for treating a renal condition in a minimally invasive manner. Certain other embodiments of the present disclosure relate to organ-selective gene delivery to the kidney using a minimally-invasive percutaneous delivery system. An exemplary method may comprise isolating a patient's renal circulation from the patient's systemic circulation and perfusing a fluid, such as a drug-containing fluid, into the patient's isolated or substantially isolated renal circulation. The perfusion may be performed in one or both kidneys, and may be used to deliver one or more drugs, including, but not limited to, gene therapy vectors, exosomes, nanoparticles, antibodies, chemotherapy, genetic medicines (e.g., mRNA, siRNA, antisense RNA), etc., without exposing the systemic circulation and, thus, other organs to the drug(s) chosen. The methods may also be used to isolate the renal circulation to allow administration, for example, of a nephrotoxic drug to the patient's systemic circulation in order to protect the kidneys from adverse effects. Isolation of the patient's renal circulation is described in more detail below with reference to FIGS. 18 and 19.

Renal conditions or diseases that may be treated by the methods disclosed herein may include, without limitations, nephronophthisis, particularly caused by autosomal recessive mutations in the NPHP1 gene, and autosomal dominant polycystic kidney disease (ADPKD), particularly caused by haploinsufficiency of the PKD1 and PKD2 genes. For example, the methods may be used to treat inherited and acquired glomerulonephritis and polycystic disease.

Nephronophthisis is an autosomal recessive kidney disease leading to end stage kidney failure. The most common form is caused by mutations, most commonly bi-allelic deletions, of NPHP1 (Hildebrandt, F. et al., Nature Genetics, vol. 17, 149-153, 1997; Saunier, S. et al., Human Molecular Genetics, vol. 6, no. 13, 2317-2323, 1997). The NPHP1 gene results in the 733 amino acid protein, nephrocystin-1 (cDNA of 2199 bases in length), located at adherens junctions and focal adhesions of renal epithelial cells that can be vectorized in an AAV. It is contemplated that substitution of nephrocystin-1 to the target tissue can alleviate or correct nephronophthisis type 1.

ADPKD affects between 1 in 400 to 1 in 1000 people and is characterized by progressive development of multiple bilateral cysts in the kidney resulting in destruction of surrounding kidney tissue and ultimately kidney failure. ADPKD accounts for 4-10% of kidney failure worldwide. ADPKD also has several systemic manifestations, including liver and pancreatic cysts, cerebral aneurysms, and cardiovascular abnormalities. Current management is limited to lifestyle changes, dialysis, and renal transplantation. Treatment with tolvaptan can slow disease progression, but is associated with significant side effects such as liver dysfunction and aquauresis.

Alport syndrome (AS) is a glomerular disease that is generally associated with sensorineural hearing loss and ocular abnormalities, and has an incidence of 1 in 5000 to 1 in 10000, with 85% of cases being associated with a mutation in the COL4A5 gene. AS can present with progressive loss of kidney function with proteinuria and hematuria, early onset hypertension, sensory deafness and ocular abnormalities. Current management approaches are limited to dialysis and renal transplantation. In AS, pathogenic variants in the genes COL4A3, COL4A4, and COL4A5 produce defective type IV collagen chains, thus hampering the assembly of glomerular basement membrane (GMB). COL4A3 and COL4A4 are located on chromosome 2, while COL4A5 is located on the X chromosome. AS is referred to as X-linked AS when the abnormality arises from a mutation in COL4A5. Although directly associated with AS, deficits of one of these three collagen genes is believed to lead to indications other than AS.

APOL1 nephropathy is a disease associated with APOL1 risk variants expression in podocytes. Patients with 2 risk alleles have been found to exhibit a more rapid decline in estimated glomerular filtration rate (eGFR). The diseases manifests as focal segmental glomerulosclerosis and hypertensive kidney diseases. Current supportive therapies are suboptimal, and include aggressive hypertension control, renin-angiotensin system blockade, steroids, interferon blockade, and conventional immunosuppressive agents.

Embodiments of the present disclosure provide for therapeutic compositions for the treatment of prevention of one or more of these diseases, discussed in greater detail below. The embodiments further provide for methods of localized transduction of the kidney with such therapeutic compositions. Transduction of solid organs by systemic administration of recombinant AAV vectors has been challenging because it requires high doses and has led to severe adverse events (SAE), particularly hepatotoxicity and thrombotic microangiopathy. Certain embodiments relate to a loco-regional delivery and perfusion system that enables the selective perfusion of solid organs. The embodiments demonstrate that targeted delivery of AAV vectors to one or both kidneys is possible without relevant discharge into the systemic circulation.

In an exemplary procedure to demonstrate the efficacy of the embodiments described herein, the left renal artery and vein of AAV-seronegative adult domestic pigs (approx. 90 kg) were catheterized percutaneously via internal jugular and femoral access. To isolate the kidney from the systemic circulation, a closed-loop was established using each animal's own heparinized blood priming (perfusate), and the loco-regional perfusion (LRP) was initiated using an extra-corporeal membrane oxygenation (ECMO) system. AAV vector with a CMV-EGFP transgene cassette was injected into the closed-loop LRP system and the loco-regional perfusion of the kidney was performed for up to 2 hours. Blood samples were collected longitudinally for safety evaluation, and vector titration and immunological assessment (e.g. complement activation, anti-AAV antibodies) was performed prior to, during, and after the procedure. After completion of the procedure, the vector-containing perfusate was withdrawn, and the catheters were removed. Animals were assessed for 2 weeks before they were euthanized and harvested for tissue processing. Quantitative PCR (qPCR) was used to detect the presence of vector genomes, and transgene expression was assessed by qPCR, Western blot, and immunohistochemistry. The procedure was successful in all animals and no peri-procedural complications occurred. The animals made a swift recovery without any clinical signs of renal injury or impairment. Vector concentration remained high and stable in the perfusate of the closed-loop throughout the procedure, with no relevant leakage to the systemic circulation or the urine. AAV particles were evenly distributed in the treated renal tissue. Green fluorescent protein (GFP) was expressed homogeneously in the perfused kidney. No vector was detected in the untreated contralateral kidney, the liver, or other organs. Anti-AAV neutralizing antibodies only mildly increased compared to baseline and no complement activation was detected. Further testing is discussed in greater detail below.

In some embodiments, the system includes an arterial access catheter that may be inserted, for example, via the arteria femoralis and sealed within the renal artery with a flow rate appropriate to perfuse and oxygenate the kidney for the duration of the procedure, typically 500-600 mL/min/kidney in a 70 kg adult (or 1000-1200 mL/1.73 m²). In some embodiments, the system includes a venous recovery catheter that may be inserted, for example, via the vena femoralis and sealed within the renal vein with a flow rate appropriate for recovery of the venous flow. In some embodiments, the system includes an extracorporeal membrane oxygenator system that fluidly connects the venous blood flow from the kidney to the arterial blood flow of the kidney, and is capable of oxygenizing the venous blood.

In some embodiments, the system includes one or more additional access lines allowing for drug administration or fluid addition. In some embodiments, a balloon catheter may be inserted into the patient's bladder to measure urine excretion during the procedure. In other embodiments, individual ureter catheters are placed in each of both ureters to differentially measure the excretion of both kidneys. In some embodiments, the system is adapted to replace a fluid volume of the perfusate that is lost due to bladder excretion. For example, in some embodiments, additional perfusate (e.g., blood) and/or other physiologically acceptable solutions (e.g., plasma or saline solution) may be used to replace about 5% v/v to about 50% v/v of the lost perfusate volume to account for bladder excretion.

In some embodiments, the system and method allow for loco-regional perfusion of one kidney with a target drug for a duration such as 15 minutes, 30 minutes, 45 minutes, one hour, 2 hours, 3 hours, 4 hours, or for any range defined therebetween. In some embodiments, the system and method allow for selective drug-targeting of one kidney or both kidneys with zero or minimal exposure of the systemic circulation and other organs to the drug. In some embodiments, a gene therapy drug may be used to treat a renal condition, which may utilize a viral vector (e.g., an adeno-associated virus), naked or encapsulated DNA or RNA molecules, synthetic DNA or RNA analogs (e.g., antisense). In some embodiments, chemotherapy may be used to target a renal tumor. In some embodiments, other drugs or biologics/antibodies may be used. In some embodiments, a combination of the aforementioned drugs may be used.

There are a number of advantages to isolating the renal circulation of the patient from the systemic circulation of the patient when treating a renal condition. These advantages include, but are not limited to: (1) loco-regional delivery of the drug, minimal leakage of the drug to other organs, and reduced overall drug dose; (2) increased targeted drug dose; (3) reduced risks and side-effects; and (4) the possibility to re-dose select patients or to dose patient populations that were not suitable therapy candidates for certain therapies (such as gene therapy with viral vectors to patients who had antibodies to the viral vectors).

Other advantages should be readily apparent to those of ordinary skill in the art. Certain embodiments relate to methods for perfusing a drug in one or both kidneys of a patient in a minimally invasive manner. Certain embodiments provide methods for circulating a perfusate (which may contain one or more of blood or a drug) through one or both kidneys of a patient such that the perfusate is isolated from the patient's systemic circulation. Certain embodiments provide loco-regional delivery of pharmaco-gene therapy. Certain embodiments can be used to reduce the overall dose of a drug delivered to a patient for treating a renal condition. Certain embodiments can be used to reduce risks and/or adverse immune response to the administration of a drug suitable for treatment of a renal condition. Certain embodiments allow for re-dosing and/or dosing a pharmaco-gene therapy drug to patients who possess neutralizing antibodies, e.g., to a gene therapy vector, that would otherwise be unsuitable candidates for receiving such drugs. Certain embodiments can be used to circulate a perfusate through the kidneys and isolate the renal circulation from the patient's systemic circulation so as to allow a potentially nephrotoxic drug to be introduced into the systemic circulation while preventing or reducing exposure of the drug to the kidneys. Certain embodiments can be used to treat renal conditions such as autosomal dominant polycystic kidney disease and nephronopthisis.

Certain embodiments can be used to provide loco-regional delivery of pharmaco-gene therapy to treat gene mutations such as mutations in the PKD1 and PKD2 genes.

### Exemplary Catheter Embodiments

Exemplary recovery catheters and perfusion catheters are now described. The catheters can be configured for the anatomy of any target organ (e.g., a kidney), for which LRP is to be performed, as would be appreciated by those of ordinary skill in the art. Moreover, it is to be understood that any of the catheters described as “recovery catheters” could also be used as “perfusion catheters,” and vice versa. The embodiments described herein are not limited to LRP of a kidney, but may also be used to isolate the circulation of the kidney from the systemic circulation, for example, to reduce or prevent exposure of the kidney to a drug or other agent introduced into the systemic circulation that may have a deleterious effect on the kidney. Those of ordinary skill in the art would appreciate other uses of the catheter embodiments described herein, for example, in applications for which sealing of a blood vessel is desired.

Embodiments of exemplary catheters for use as recovery catheters in an LRP system are now described. In at least one embodiment, the recovery catheters are designed to support a liquid suction flow rate of about 400 mL/min or greater (e.g., about 700 mL/min or greater). For example, in at least one embodiment, an exemplary catheter can support an in vitro suction flow rate of about 800 mL/min at about −80 mmHg.

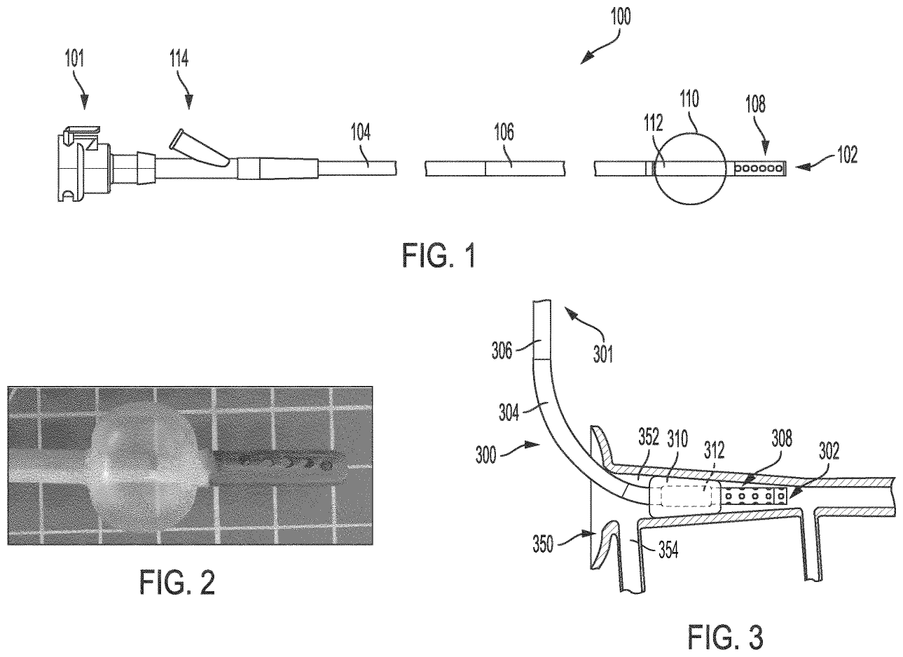

FIGS. 1-10 depict various catheter embodiments suitable for fluid recovery in an LRP system. Any of the catheters depicted in FIGS. 1-10 may be configured to support liquid flow rates (suction or perfusion) of at least about 400 mL/min, at least about 450 mL/min, at least about 500 mL/min, at least about 550 mL/min, at least about 600 mL/min, at least about 650 mL/min, at least about 700 mL/min, at least about 750 mL/min, at least about 800 mL/min, at least about 850 mL/min, at least about 900 mL/min, at least about 950 mL/min, or at least about 1000 mL/min. Each catheter may be compatible with a stearable introducer sheath, which provides stability and directs the distal end of the catheter, and allows for the catheter to create a directed push force. Each catheter may also have a pull wire integrated into its shaft assembly, allowing for sections proximal to the occlusion structure to bend at angles of up to 120° and achieve better tracking and centering of the occlusion structure.

In at least one embodiment, one or more of the catheters may be multi-lumen catheters, such as double-lumen catheters. In at least one embodiment, the multi-lumen catheters allow for liquid flow (e.g., a perfusate) and enable inflation of one or more balloons. In at least one embodiment, one or more of the catheters may be multi-balloon catheters having two or more balloons. In at least one embodiment, one or more of the balloons may be deployed or deflated independently.

FIG. 1 illustrates an exemplary catheter 100 having a lumen shaft 104/106 with a proximal end 101 and a distal end 102. The lumen shaft 104/106 can be formed from an outer lumen shaft 104 that at least partially encompasses an inner lumen shaft 106 to expose a distal portion of the inner lumen shaft 106 near the distal end 102. The proximal end 101 includes an outlet structure that can be fluidly coupled to an LRP system. One or more of the outer lumen shaft 104 or the inner lumen shaft 106 may be formed from a durable polymer material such as a polyether block amide (PEBA) material (e.g., commercially available as PEBAX®). In at least one embodiment, an innermost diameter (“inner diameter”) of the inner lumen shaft 106 is at least about 4 mm to provide a liquid flow path. In at least one embodiment, the catheter 100 may be designed to include additional lumen shafts.

The catheter 100 includes a tip portion 108 at the distal end 102 and an expandable balloon structure 110 disposed along a portion 112 of the inner lumen shaft 106. In at least one embodiment, the tip portion 108 includes an elongated shaft extending from the balloon structure 110 to the distal end 102. In at least one embodiment, the length of the elongated shaft of the tip portion is from about 2 mm to about 35 mm, about 5 mm to about 30 mm, about 10 mm to about 25 mm, about 15 mm to 25 mm, or within any subrange defined between (e.g., about 2 mm to about 5 mm). In at least one embodiment, the tip portion 108 includes an opening at the distal end 102 and one or more perforations along the elongated shaft. In at least one embodiment, the tip portion is formed from a compliant material that is more flexible than the material of the inner lumen shaft 106.

In at least one embodiment, the inner lumen shaft 106 includes a concentric inner flow path surrounding the liquid flow path. The concentric inner flow path provides a path for gas flow from the balloon structure 110 to a port 114, which can be used to inflate or deflate the balloon depending on the pressure applied at the port 114. In at least one embodiment, an outermost surface of the inner lumen shaft 106 at the portion 112 is removed such that the portion 112 is sealed by the balloon structure 110 to isolate gas flow from the concentric inner flow path to the balloon structure 110. In at least one embodiment, an expanded diameter of the balloon structure is from about 15 mm to about 30 mm, about 15 mm to about 20 mm, about 20 mm to about 25 mm, about 24 mm to about 28 mm, or about 25 mm to about 30 mm.

FIG. 2 is an image of a catheter having a similar structure to the catheter 100 with a balloon in its deployed state. The dimensions of the catheter include: a crossing profile of 19 Fr (6.3 mm); an innermost diameter of 12 Fr (4.0 mm); a usable length of 80 cm; a balloon diameter (when deployed) of 25 mm; and a tip portion length of 20 mm. The lumen shaft can be formed from a polymer material such as PEBAX® 63 that is supported by a strong stainless-steel braid. The balloon can be formed from a compliant thermoplastic/elastomeric material such as ChronoPrene™ 25A. The tip portion can be formed from a polymer material such as PEBAX® 35 and can be loaded with a radio marker or a radiopaque filler composition, such as BaSO₄.

FIG. 3 illustrates insertion of an exemplary catheter 300 into a vessel 352 via a larger vessel or chamber 350 (referred to herein as a “vessel”) according to at least one embodiment. In the anatomy depicted, blood flow from the vessels 352 and 354 drain into the vessel 350. The catheter 300 may be the same as or similar to the catheter 100, having a proximal end 301, a distal end 302, an inner lumen shaft 304, an outer lumen shaft 306, a tip portion 308, and a balloon structure 310 disposed on a portion 312 of the inner lumen shaft 304. The balloon structure 310 when deployed is compliant enough to adapt to the anatomy of the vessel 352 and occlude the blood flow through the vessel 352 into the vessel 350 without creating excessive force on the tissue. As illustrated in FIG. 3, the catheter 300 is inserted past the vessel 354 so as to avoid occluding the flow from the vessel 354 into the vessel 350.

It is noted that the vessel or chamber 350, the vessel 352, and the vessel 354 are illustrative of the anatomy of, respectively, the right atrium, the coronary sinus, and the middle cardiac vein of a heart to illustrate various types of occlusion techniques for which the exemplary catheters can be utilized. However, they are referred to herein as generic vessels as it is to be understood that the deployment of any of the catheters described herein may be adapted to specific anatomies for target organs (e.g., a kidney) in which LRP or occlusion is to be performed. For example, the vessel 350 and the vessel 352 may correspond, respectively, to the inferior vena cava and the renal vein of a kidney (without the presence of the vessel 354).

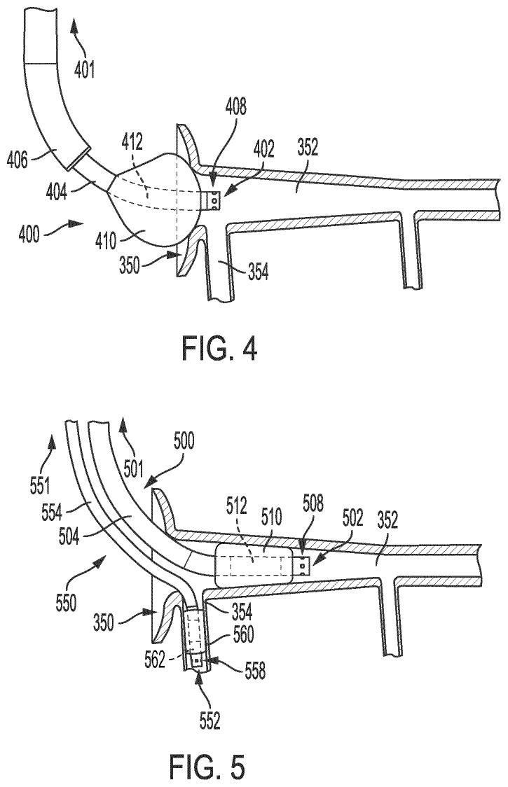

FIGS. 4-10 illustrate other occlusion techniques in accordance with various embodiments of the disclosure. The catheters depicted in FIGS. 4-10 may be similar in certain aspects to the catheters depicted in FIGS. 1-3, for example, in terms of dimensions, materials, or structures.

FIG. 4 illustrates a catheter 400 according to at least one embodiment that is only partially inserted into the vessel 352 such that it abuts the ostium of the vessel 352. The catheter 400 includes a proximal end 401, a distal end 402, an inner lumen shaft 404, an outer lumen shaft 406, a tip portion 408, and a balloon structure 410 disposed on a portion 412 of the inner lumen shaft 404. In at least one embodiment, a diameter of the balloon structure 410 is greater than about 15 mm, greater than about 20 mm, greater than about 25 mm, or greater than about 30 mm when deployed. The tip portion 408 may include, in addition to an opening at the distal end 402, one or more perforations to facilitate flow of blood from the vessel 352 and the vessel 354 into the catheter 400.

In at least one embodiment, during deployment, the outer lumen shaft 406 can be moved distally to abut against the deployed balloon structure 410, resulting in additional pressure by the balloon structure 410 against the ostium of the vessel 352 to further stabilize the position of the catheter 400. In at least another embodiment, a wire structure may be utilized to apply pressure to the balloon structure 410. The wire structure, for example, may have a sinusoidal shape that is deployable to an expanded flower-like structure extending radially from the outer lumen shaft 406 or the inner lumen shaft 404. When brought into contact with the balloon structure 410, the wire structure may produce a more even pressure profile across the surface of the balloon structure 410. Prior to deployment, the wire structure may be covered by the outer lumen shaft 406, or may be covered by an additional lumen outside of the outer lumen shaft 406.

FIG. 5 illustrates the use of a first catheter 500 and a second catheter 550 for separately occluding and draining the vessel 352 and the vessel 354, respectively, according to at least one embodiment. The first catheter 500 includes a proximal end 501, a distal end 502, a lumen shaft 504, a tip portion 508, and a balloon structure 510 disposed on a portion 512 of the lumen shaft 504. Similarly, the second catheter 550 includes a proximal end 551, a distal end 552, a lumen shaft 554, a tip portion 558, and a balloon structure 560 disposed on a portion 562 of the lumen shaft 554. In this configuration, the first catheter 500 is inserted into the vessel 352 such that the balloon structure 510 does not occlude the vessel 354, while the second catheter 550 is inserted directly into the vessel 354. The dimensions of the first catheter 500 and the second catheter 550 may be selected to provide safe and effective occlusion of the vessel 352 and the vessel 354, respectively.

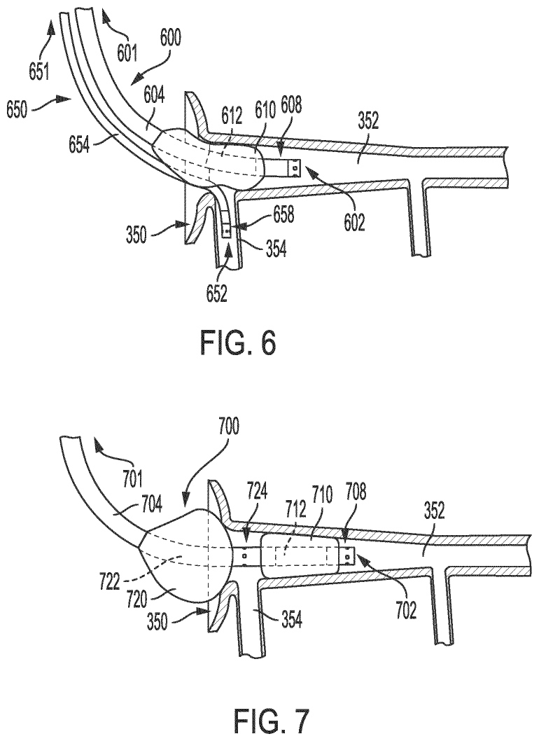

FIG. 6 illustrates a variation of FIG. 5, which uses two catheters with only one having a balloon structure according to at least one embodiment. A first catheter 600 includes a proximal end 601, a distal end 602, a lumen shaft 604, a tip portion 608, and a balloon structure 610 disposed on a portion 612 of the lumen shaft 604. A second catheter 650 includes a proximal end 651, a distal end 652, a lumen shaft 654, and a tip portion 658, and does not include a balloon structure. The first catheter 600 is inserted into the vessel 352 such that a portion of the balloon structure 610 occludes the vessel 354 and is partially within the vessel 350 and the vessel 352. The second catheter 650 is inserted directly into the vessel 354 and is disposed between the vessel wall and the balloon structure 610, which at least partially occludes the vessel 354.

FIG. 7 illustrates the use of a single catheter 700 which includes multiple balloons according to at least one embodiment. The catheter 700 includes a proximal end 701, a distal end 702, a lumen shaft 704, a tip portion 708, a first balloon structure 710 disposed on a first portion 712 of the lumen shaft 704, and a second balloon structure 720 disposed on a second portion 722 of the lumen shaft 704. In at least one embodiment, the catheter 700 is designed for insertion into the vessel 352 such that the first balloon structure 710 occludes the vessel 352, and the second balloon structure 720 abuts the ostium of the vessel 352 to occlude the vessel 354 (and further occlude the vessel 352). An intermediate portion 724 of the lumen shaft 704 between the first balloon structure 710 and the second balloon structure 720 includes one or more perforations to allow drainage of the vessel 354. In at least one embodiment, an expanded diameter of the second balloon structure 720 is greater than an expanded diameter of the first balloon structure 710. In at least one embodiment, the catheter 700 is a multi-lumen catheter designed to allow each balloon to be deployed and deflated independently of each other.

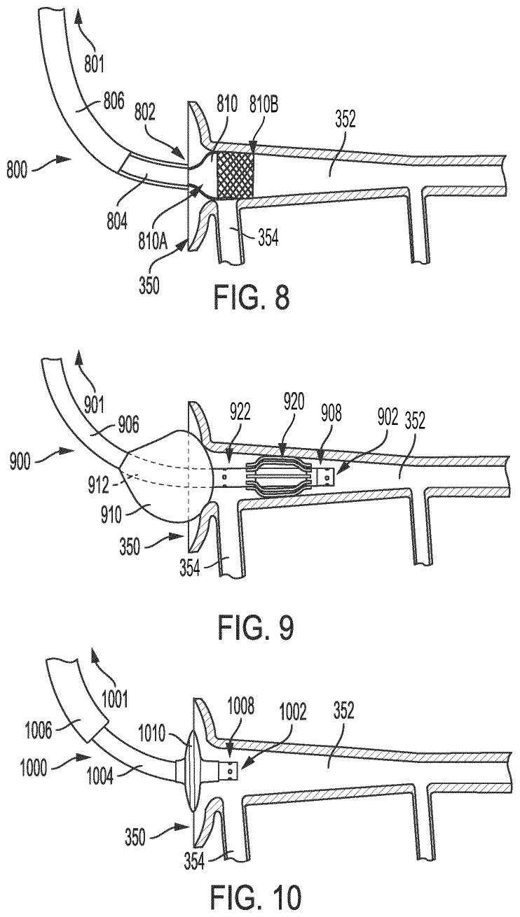

FIG. 8 illustrates a catheter 800 that includes a partially covered and recapturable stent structure 810 according to at least one embodiment. The catheter 800 includes a proximal end 801 and a distal end 802, an inner lumen shaft 804 coupled to the stent structure 810, and an outer lumen shaft 806. Part of the outer lumen shaft 806 is depicted as a cutaway view to illustrate the inner lumen shaft 804 within. The stent structure 810 is depicted in its deployed state, but can be contained within the outer lumen shaft 806 prior to deployment. The stent structure 810 is further depicted as having a proximal covered portion 810A, which may be formed from a flexible and durable polymer material, and a distal uncovered portion 810B. When inserted into the vessel 352, as shown, the covered portion 810A occludes blood flow out of the vessel 352, while the uncovered portion 810B provides structural support within the vessel 352 while allowing blood flow from both the vessel 352 and the vessel 354 directly into the catheter 800. In at least one embodiment, the catheter 800 can be used as a perfusion catheter connected to a supply line.

FIG. 9 illustrates a catheter 900 that includes a deployable and retractable stent structure 920 according to at least one embodiment. The catheter 900 further includes a proximal end 901, a distal end 902, a lumen shaft 906, a tip portion 908, and a balloon structure 910 disposed on a portion 912 of the lumen shaft 906. The catheter 900 can further include an outer lumen shaft (not shown) that substantially encapsulates the stent structure 920 and the balloon structure 910 prior to deployment. Deployment of the stent structure 920 can be performed by moving the outer lumen shaft in a proximal direction, and retraction of the stent structure 920 can be performed by moving the outer lumen shaft in a distal direction. The stent structure 920 may be formed from, for example, stainless-steel, and is disposed between the balloon structure 910 and the tip portion 908. In at least one embodiment, the lumen shaft 906 comprises at least one perforation along a portion 922 between the balloon structure 910 and the stent structure 920 to allow drainage of the vessel 354 into the catheter 900. When inserted into the vessel 352, the balloon structure 910 abuts the ostium of the vessel 352.

FIG. 10 illustrates a catheter 1000 that includes a covered disk-shaped stent structure 1010 according to at least one embodiment. The catheter 1000 further includes a proximal end 1001, a distal end 1002, an outer lumen shaft 1006, an inner lumen shaft 1004, and a tip portion 1008. The stent structure 1010 may be formed from, for example, a stainless-steel stent having a durable polymer covering. The outer lumen shaft 1006 can cover the stent structure 1010 prior to deployment. Once the catheter 1000 is properly positioned, the outer lumen shaft 1006 can be moved in the proximal direction to enable deployment of the stent structure 1010. In at least one embodiment, the stent structure 1010 is coupled to the tip portion 1008, which may be partially contained within the inner lumen shaft 1004 and can be actuatable (using a wire) to deploy the stent structure 1010 when moved in a proximal direction and retract the stent structure 1010 when moved in a distal direction. In at least one embodiment, the stent structure 1010, when deployed, is large enough to occlude the vessel 352 and the vessel 354 when abutted to the ostium of the vessel 352. In at least one embodiment, a diameter of the stent structure 1010 is from about 10 mm to about 30 mm.

Embodiments of exemplary catheters for use as perfusion catheters in an LRP system are now described. In at least one embodiment, the perfusion catheters are designed to support a liquid perfusion flow rate of about 400 mL/min or greater (e.g., about 700 mL/min or greater). In embodiments that utilize multiple perfusion catheters can support a combined flow capacity of 700 mL/min or greater.

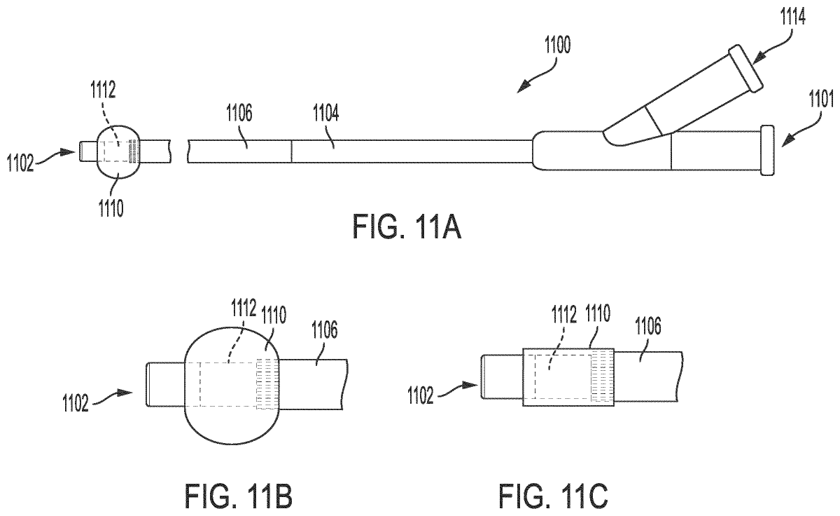

FIGS. 11-16 depict various catheter embodiments suitable for fluid perfusion in an LRP system. Any of the catheters depicted in FIGS. 11-16 may be configured to support liquid flow rates (suction or perfusion) of at least about 400 mL/min, at least about 450 mL/min, at least about 500 mL/min, at least about 550 mL/min, at least about 600 mL/min, at least about 650 mL/min, at least about 700 mL/min, at least about 750 mL/min, at least about 800 mL/min, at least about 850 mL/min, at least about 900 mL/min, at least about 950 mL/min, or at least about 1000 mL/min. Each catheter can be designed to have a smooth profile from a proximal catheter body to a low distal profile, for example, using one or more concentric lumen shafts. In addition, the catheters can be designed to have lumen shafts that are pre-shaped depending on the anatomy in which the LRP procedure is to be performed, which may improve overall stability during use.

In at least one embodiment, one or more of the catheters may be multi-lumen catheters, such as double-lumen catheters. In at least one embodiment, the multi-lumen catheters allow for liquid flow (e.g., a perfusate) and enable inflation of one or more balloons. In at least one embodiment, one or more of the catheters may be multi-balloon catheters having two or more balloons. In at least one embodiment, one or more of the balloons may be deployed or deflated independently.

FIGS. 11A-11C illustrate an exemplary catheter 1100 having a lumen shaft 1104/1106 with a proximal end 1101 and a distal end 1102 having an opening from which a perfusate can flow. The lumen shaft 1104/1106 can be formed from an outer lumen shaft 1104 that at least partially encompasses an inner lumen shaft 1106 to expose a distal portion of the inner lumen shaft 1106 near the distal end 1102. The proximal end 1101 includes an outlet structure that can be fluidly coupled to an LRP system. One or more of the outer lumen shaft 1104 or the inner lumen shaft 1106 may be formed from a durable polymer material such as a polyether block amide (PEBA) material (e.g., commercially available as PEBAX®). In at least one embodiment, an innermost diameter of the inner lumen shaft 1106 is at least about 2 mm, at least about 2.5 mm, at least about 3 mm, at least about 3.5 mm, at least about 4 mm, at least about 4.5 mm, or at least about 5 mm to provide a liquid flow path.

The catheter 1100 includes an expandable balloon structure 1110 disposed along a portion 1112 corresponding to the inner lumen shaft 1106 and a tip portion formed by an additional lumen. In at least one embodiment, the inner lumen shaft 1106 includes a concentric inner flow path surrounding the liquid flow path. The concentric inner flow path provides a path for gas flow from the balloon structure 1110 to a port 1114, which can be used to inflate or deflate the balloon structure 1110 depending on the pressure applied at the port 1114. In at least one embodiment, an outermost surface of the inner lumen shaft 1106 at the portion 1112 is removed such that the portion 1112 is sealed by the balloon structure 1110 to isolate gas flow from the concentric inner flow path to the balloon structure 1110. In at least one embodiment, an expanded diameter of the balloon structure 1110 is from about 15 mm to about 30 mm, about 15 mm to about 20 mm, about 20 mm to about 25 mm, about 24 mm to about 28 mm, about 25 mm to about 30 mm, or within any subrange defined therebetween (e.g., about 20 mm to about 28 mm). FIGS. 11B and 11C illustrate the balloon structure 1110 in its deployed and deflated states.

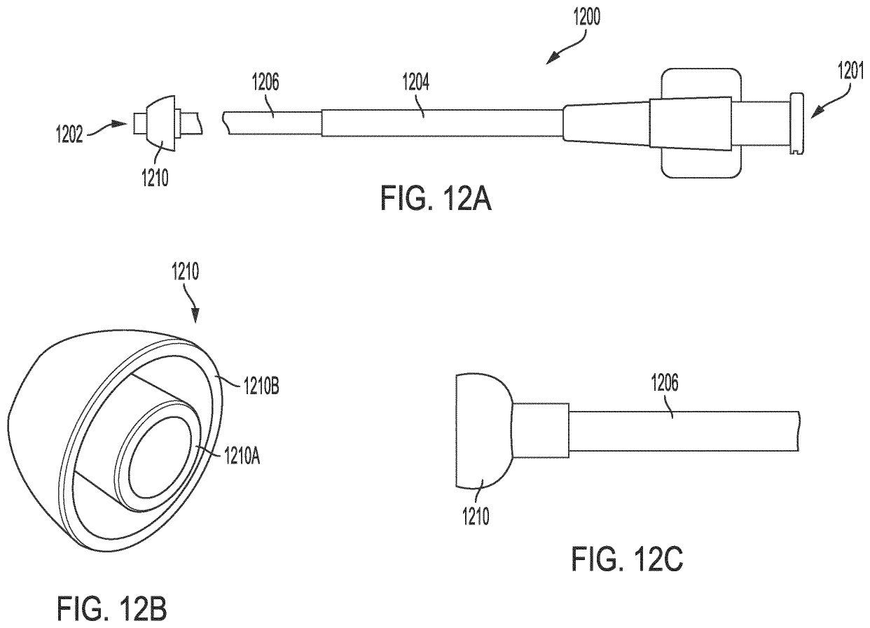

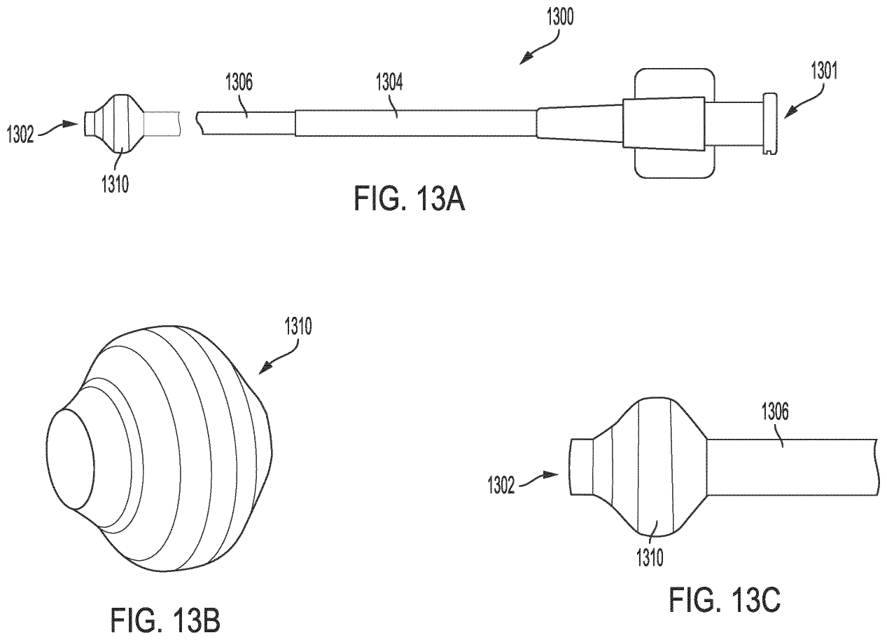

FIGS. 12 and 13 illustrate catheters that include plug and wedge occlusion structures, respectively, that advantageously adapt their shapes to a vessel or ostium, are formed from highly compressible and atraumatic materials for safe introduction and deployment, are shorter in length in comparison to a balloon structure, and do not require an additional lumen for inflation as would a balloon structure.

FIGS. 12A-12C illustrate an exemplary catheter 1200 having a lumen shaft 1204/1206 with a proximal end 1201 and a distal end 1202 having an opening from which a perfusate can flow. The lumen shaft 1204/1206 can be formed from an outer lumen shaft 1204 that at least partially encompasses an inner lumen shaft 1206 to expose a distal portion of the inner lumen shaft 1206 near the distal end 1202. The proximal end 1201 includes an outlet structure that can be fluidly coupled to an LRP system. One or more of the outer lumen shaft 1204 or the inner lumen shaft 1206 may be formed from a durable polymer material such as a polyether block amide (PEBA) material (e.g., commercially available as PEBAX®). In at least one embodiment, an innermost diameter of the inner lumen shaft 1206 is at least about 2 mm, at least about 2.5 mm, at least about 3 mm, at least about 3.5 mm, at least about 4 mm, at least about 4.5 mm, or at least about 5 mm to provide a liquid flow path.

The catheter 1200 further includes a plug 1210 near the distal end 1202. In at least one embodiment, the plug 1210 is formed from a flexible material, such as silicone or a foam material. In at least one embodiment, the plug 1210 includes an inner portion 1210A that fits onto the inner lumen shaft 1206 and a flexible outer portion 1210B shaped to be configurable between a retracted state (FIG. 12A) and an extended state (FIG. 12C) for which the outer portion 1210B extends distally from the distal end 1202. The plug 1210 in FIG. 12A is illustrated as tapering in a distal direction. In at least one embodiment, the plug 1210 may be reversed such that it tapers in a proximal direction. In at least one embodiment, the outer lumen shaft 1204 may be configured to cover the plug 1210 prior to deployment. When utilized as a perfusion catheter, the pressure of arterial blood flow into the hollow space between the inner portion 1210A and the outer portion 1210B of the plug 1210 can help improve the sealing of the catheter 1200 within the vessel in which it is deployed.

FIGS. 13A-13C illustrate an exemplary catheter 1300 having a lumen shaft 1304/1306 with a proximal end 1301 and a distal end 1302 having an opening from which a perfusate can flow. The lumen shaft 1304/1306 can be formed from an outer lumen shaft 1304 that at least partially encompasses an inner lumen shaft 1306 to expose a distal portion of the inner lumen shaft 1306 near the distal end 1302. The proximal end 1301 includes an outlet structure that can be fluidly coupled to an LRP system. One or more of the outer lumen shaft 1304 or the inner lumen shaft 1306 may be formed from a durable polymer material such as a polyether block amide (PEBA) material (e.g., commercially available as PEBAX®). In at least one embodiment, an innermost diameter of the inner lumen shaft 1306 is at least about 2 mm, at least about 2.5 mm, at least about 3 mm, at least about 3.5 mm, at least about 4 mm, at least about 4.5 mm, or at least about 5 mm to provide a liquid flow path.

The catheter 1300 further includes a wedge 1310 near the distal end 1302, which may be shaped to adapt to a vessel or ostium. In at least one embodiment, the wedge 1310 is formed from a flexible material, such as silicone or a foam material. In at least one embodiment, the outer lumen shaft 1304 may be configured to cover the wedge 1310 prior to deployment. When deployed in a vessel, the shape of the wedge can leverage back-up forces from the vessel wall to further enhance stability during occlusion and perfusion of the vessel.

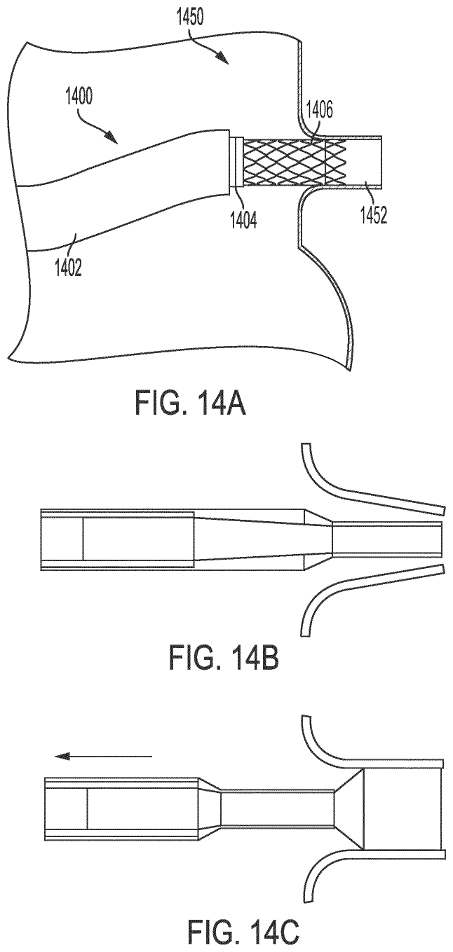

FIGS. 14A-14C illustrate an exemplary catheter 1400 that includes a partially covered and recapturable stent structure 1406 in accordance with at least one embodiment, similar to the catheter 800 described with respect to FIG. 8. The catheter 1400 is illustrated as being inserted into an arterial vessel 1452 via a vessel or chamber 1450. The catheter 1400 includes an outer lumen shaft 1402 and an inner lumen shaft 1404 that is coupled to the stent structure 1406 in at least one embodiment. The stent structure 1406 is further depicted as having a proximal covered portion, which may be formed from a flexible and durable polymer material, and a distal uncovered portion. FIGS. 14B and 14C illustrate placement and deployment, respectively, of the stent structure 1406 when inserted into the vessel 1452. Deployment of the stent structure 1406 is performed by moving the outer lumen shaft 1402 in the proximal direction.

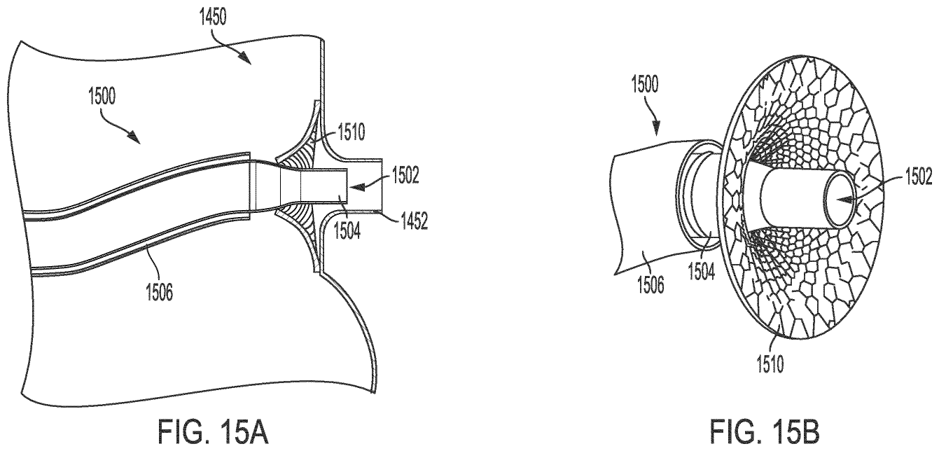

FIGS. 15A and 15B illustrate an exemplary catheter 1500 that includes a releasable covered braided disk 1510, in accordance with at least one embodiment. The catheter 1500 includes an outer lumen shaft 1506 and an inner lumen shaft 1504. The braided disk 1510 is contained within the outer lumen shaft 1506 during placement of the catheter 1500, and can be deployed by moving the outer lumen shaft 1506 in the proximal direction. In at least one embodiment, when deployed, the braided disk 1510 does not expand past the distal end 1502, and is used to stabilize the catheter 1500 against the ostium of the vessel 1452 to reduce the risk of stenosis during occlusion of the vessel 1452, while allowing the distal end 1502 to extend into the vessel 1452.

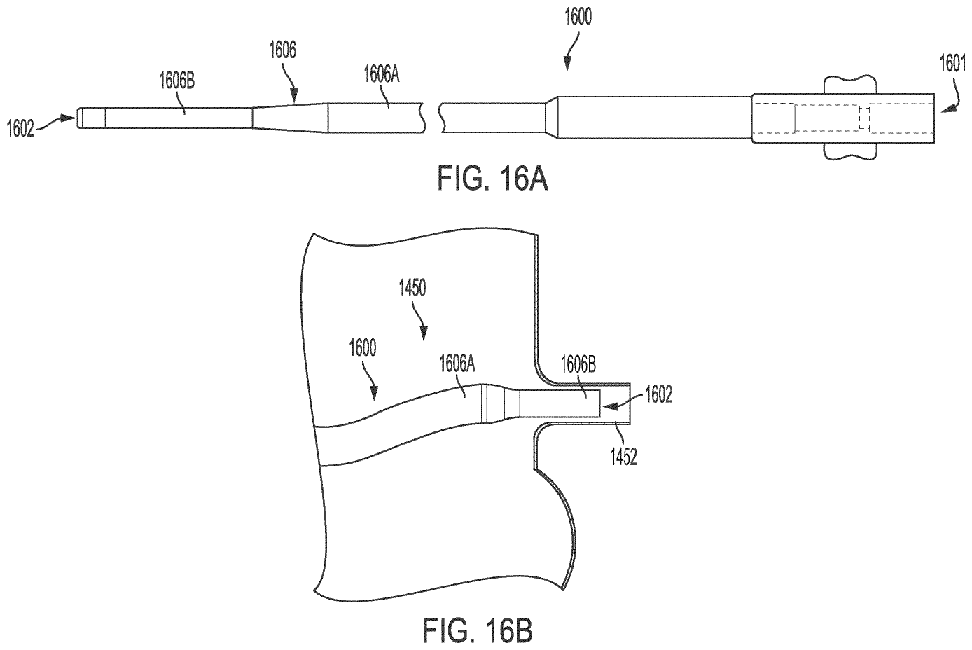

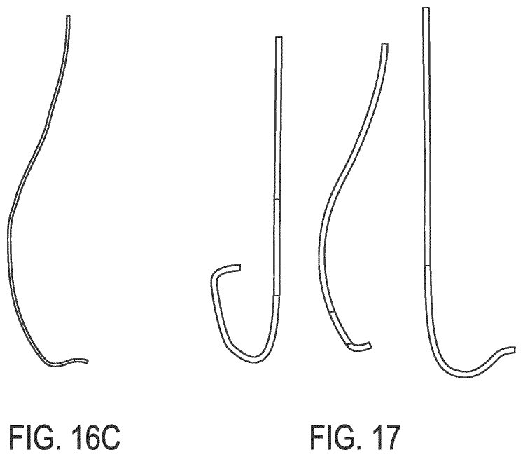

FIGS. 16A-16C illustrate an exemplary catheter 1600 having a lumen shaft 1606 with a proximal end 1601 and a distal end 1602 having an opening from which a perfusate can flow. The proximal end 1601 includes an outlet structure that can be fluidly coupled to an LRP system. The lumen shaft 1604 may be formed from a durable polymer material such as a polyether block amide (PEBA) material (e.g., commercially available as PEBAX®). In at least one embodiment, an innermost diameter of the lumen shaft 1606 is at least about 2 mm, at least about 2.5 mm, at least about 3 mm, at least about 3.5 mm, at least about 4 mm, at least about 4.5 mm, or at least about 5 mm to provide a liquid flow path. In at least one embodiment, a proximal portion 1606A of the lumen shaft 1606 may have a larger diameter than a distal portion 1606B of the lumen shaft 1606, and can taper gradually over a length of the lumen shaft 1606. FIG. 16C illustrates the lumen-shaft in a pre-shaped form to facilitate introduction and placement into a vessel of a target organ.

Examples of pre-shaped catheter lumens are illustrated in FIG. 17. The catheter lumens can be shaped to abut regions of the anatomy when deployed, utilizing back-up forces from the vessel walls to further enhance stability during occlusion and perfusion of the target organ.

### Exemplary LRP System Embodiments

FIG. 18 depicts an exemplary LRP system 1800 in accordance with embodiments of the present disclosure. The LRP system 1800 is shown in a closed circuit configuration with a kidney 1810. The LRP system 1800 includes a membrane oxygenation device 1820, a blood gas analysis (BGA) monitor 1830, a fluid source 1840, a flow measurement device 1842, an ECMO pump console 1846 to monitor and control fluid flow, and a pressure wire and console 1844 to measure pressure within the closed circuit. In at least one embodiment, a vacuum pump 1848 may also be utilized. The LRP system 1800 may be assembled by positioning a first catheter 1822 (which may be referred to herein as a “perfusion catheter”) in the renal artery of the kidney 1810, and positioning a second catheter 1824 (which may be referred to herein as a “recovery catheter,” a “collection catheter,” or a “suction catheter”) in the renal vein of the kidney 1810. The first catheter 1822 and the second catheter 1824 together with the vasculature of the kidney 1810, the membrane oxygenation device 1820, and one or more optional additional components form a closed circuit. This closed circuit may isolate or substantially isolate the renal circulation of the patient from the systemic circulation of the patient.

The first catheter 1822 and the second catheter 1824 may be introduced percutaneously and in a minimally invasive manner. In some embodiments, the first catheter 1822 and/or the second catheter 1824 may be introduced via antegrade intubation. In other embodiments, the first catheter 1822 and/or the second catheter 1824 may be introduced via retrograde intubation. The first catheter 1822 may be referred to herein as a “drug delivery catheter” and the second catheter 1824 may be referred to herein as a “drug collection catheter” when the catheters are used for drug delivery to the kidney or kidneys.

The first catheter 1822 may be a standard infusion catheter that may optionally include a standard guidewire and infusion pump, and is capable of delivering a perfusate to the kidney 1810, which may contain, for example, a drug to be delivered to the kidney 1810 during loco-regional perfusion. In some embodiments, the first catheter 1822 is positioned in the renal artery via the arteria femoralis. In some embodiments, the second catheter 1824 is positioned in the renal vein via the vena femoralis. In some embodiments, the second catheter 1824 is a balloon catheter such that the balloon may be inflated within the renal vein to ensure that all the blood circulated through the closed circuit flows through the second catheter 1824. The balloon catheter may be a Fogarty® catheter, or any other catheter suitable for the purposes discussed herein as will be appreciated by one of ordinary skill in the art. In some embodiments, the first catheter 1822 and the second catheter 1824 may each be a balloon catheter to help reduce leakage. In some embodiments, any of the catheters may be selected from one or more of the catheters described with respect to FIGS. 1-17.

The LRP system 1800 may further comprise one or more additional components, such as, without limitations, one or more pumps (e.g., the vacuum pump 1848), one or more suction mechanisms, one or more perfusates, and combinations thereof. For example, the LRP system 1800 may include the pressure wire and console 1844, which in some embodiments is operatively coupled to or part of the membrane oxygenation device 1820. The pressure wire and console 1844 and the ECMO pump console 1846 may collectively be used to control the perfusion rate (i.e., flowrate) and ensure safety by continuously monitoring the renal artery pressure. A first pressure sensor and a second pressure sensor, for example, may be co-inserted with the first catheter 1822 and the second catheter 1824, respectively, to measure the pressures within the renal artery and the renal vein, respectively. The LRP system 1800 is further depicted as including a BGA monitor 1830 that is operatively coupled to the membrane oxygenation device 1820 to measure, for example, the gas concentrations in the perfusate (e.g., when the perfusate contains blood) prior to perfusion via the first catheter 1822 and/or after the perfusate is collected by the second catheter 1824. The membrane oxygenation device 1820 and one or more additional components may be placed between the first catheter 1822 and the second catheter 1824.

In some embodiments, the LRP system 1800 includes a third catheter 1826 for draining the bladder 1812. In some embodiments, the third catheter 1826 is a balloon catheter to block the leakage of fluid from the bladder 1812. A flow measurement device 1842 may be used to measure an excreted volume of urine from the bladder 1812 during the LRP procedure. In some embodiments, a fluid source 1840 may be used to replace the volume of excreted fluid that is lost from the perfusate by injecting the fluid into the closed circuit via a fluid line 1841. In some embodiments, the fluid is the same as the perfusate, or has less than all components of the perfusate (e.g., without additional drug). In some embodiments, the fluid is a physiologically acceptable solution (e.g., a saline solution).

In some embodiments, the LRP system 1800 may be modified so as to simultaneously establish closed circuits within each of the patient's kidneys. In some embodiments, two separate LRP systems may be used for each of the patient's kidneys.

In at least one embodiment, the LRP system 1800 may be modified to include multiple perfusion or recovery catheters. For example, the first catheter 1822 may be supplemented with one or more additional perfusion catheters that are fluidly coupled to the ECMO pump console 1846 (e.g., directly or via a split supply line) such that each catheter can be placed within different locations of the kidney 1810 vasculature. Similarly, the second catheter 1824 may be supplemented with one or more additional recovery catheters that are fluidly coupled to the ECMO pump console 1846.

In some embodiments, while the closed circuit is established, one or more drugs may be perfused through the patient's systemic circulation. For example, if the drug is nephrotoxic or potentially harmful to the kidneys but systemic delivery is desirable, establishing closed circuits through the kidneys to isolate the renal perfusion from the systemic perfusion is advantageous in preventing or reducing exposure of the drug to the kidneys.

FIG. 19 is a schematic of the membrane oxygenation device 1820, which may be used to oxygenate the perfusate, mix the perfusate with other components (e.g., a drug), remove carbon dioxide from the perfusate, and/or push the perfusate into the first catheter 1822. The membrane oxygenation device 1820 may be any commercially available ECMO device for exchanging oxygen for carbon dioxide contained in the blood.

As illustrated in FIG. 19, the membrane oxygenation device 1820 includes various components including a heat exchanger 1856 (through which the perfusate passes prior to leaving an outlet 1852 and entering the first catheter 1822), a delivery pump 1858, a reservoir 1860 (for adding a component, such as blood and/or a drug, to the perfusate returning through the second catheter 1824 through an inlet 1854), sensors 1862 and 1864 at various stages of the closed circuit (e.g., for measuring pressure and/or blood gas content), and a membrane oxygenator 1866. In some embodiments, de-oxygenated blood enters the membrane oxygenator 1866 and is mixed with an oxygen-rich gas. The oxygen-rich gas may be supplied from a gas blender 1868 that may mix oxygen in various ratios with carbon dioxide and nitrogen gas, and is regulated by a gas regulator 1870.

The perfusate may comprise one or more of blood (or its components such as plasma or serum) and/or drug suitable for treatment of the renal condition and/or a vehicle such as saline or dextrose solutions. The delivery pump 1858 may deliver the perfusate into the first catheter 1822. In some embodiments, the perfusate may be contained in an IV bag or a syringe and may be administered directly to the first catheter 1822 with or without the delivery pump 1858.

A suction mechanism may be used to apply negative suction pressure on the second catheter 1824 to minimize blood and/or drug leakage outside of the closed circuit. The negative suction pressure may be about −150 mmHg, about −100 mmHg, about −50 mmHg, about −20 mmHg, about −15 mmHg, about −10 mmHg, about −5 mmHg, 0 mmHg, or within a subrange defined by any of these points.

Blood circulated through the closed circuit may be autologous blood, matched blood from donors, or a combination thereof. In some embodiments, blood components, such as serum or plasma, are chosen according to one or more parameters. One of the parameters may be the presence or absence of selected antibodies. For instance, when the drug is one or more viral vectors encompassing a therapeutic nucleic acid sequence, the patient's autologous blood may be screened to determine whether antibodies to the one or more viral vectors are present. Presence of antibodies in the patient's autologous blood may reduce and/or negate altogether the effectiveness of the treatment and/or may result in an undesirable immune response. As such, it may be possible to dilute or replace the patient's autologous blood with a seronegative matched blood from donors, thereby reducing a patient's immune response to the drug and enhancing the effectiveness of the drug.

While the various components illustrated in FIG. 19 show components that are part of or separate from the membrane oxygenation device 1820, it is to be understood that this schematic is merely illustrative, as one or more of the components may be included in or separate (external) from the membrane oxygenation device 1820.

The LRP system 1800 may be set up and operated as follows: (1) a recovery catheter (e.g., the second catheter 1824) is carefully placed and tightly sealed in the renal vein to enable the collection of de-oxygenated venous blood; (2) a perfusion catheter (e.g., the first catheter 1822) is placed in the renal artery in a sealed fashion; (3) an additional recovery catheter (e.g., the third catheter 1826) is inserted into the bladder, into the ureter, or both in a sealed fasion; (4) the perfusion and recovery catheters are then connected to arterial and venous lines of the membrane oxygenation device 1820 using standard tubes; (5) operation of the LRP system 1800 is started, and the renal artery is antegradely perfused with oxygenated blood, while the returning de-oxygenated blood is collected from the renal vein via the recovery catheter using gentle negative pressure; (6) blood is then directed into the reservoir 1860 and is subsequently oxygenated by the membrane oxygenator 1866 and antegradely re-infused (driven by the delivery pump 1858) into the kidney via the first catheter 1822; and (7) fluid volume excreted through the bladder is then measured using the flow measurement device 1842 and is replaced in the perfusate by the fluid source 1840. If a drug (e.g., a vector) is administered, this can be added into the perfusate via the reservoir 1860 after priming with blood or plasma, and blood samples can be taken, or drugs can be applied via the reservoir 1860 during the entire perfusion process.

In some embodiments, diluting or replacing a patient's antibody-containing autologous blood with a seronegative matched blood from donors (e.g., exchanging the volume circulating in the system by removing venous blood and flushing in antibody-free blood to reduce the amount of circulating antibodies specific to the viral vector used) may result in a reduced adverse immune response and/or improved drug efficacy. For instance, the adversity of a patient's immune response may be reduced by about 10%, by about 20%, by about 30%, by about 40%, by about 50%, by about 60%, by about 70%, by about 80%, by about 90%, or alleviated altogether, upon dilution or replacement of autologous blood with seronegative matched blood from donors as compared to a patient's immune response without autologous blood dilution or replacement. The efficacy of a drug administered may be increased by about 10%, by about 20%, by about 30%, by about 40%, by about 50%, by about 60%, by about 70%, by about 80%, by about 90%, by about 100%, by about 150%, by about 200%, by about 300%, by about 400%, or by about 500%, upon dilution or replacement of autologous blood with seronegative matched blood from donors as compared to the drug's efficacy in a patient without autologous blood dilution or replacement.

In some embodiments, the blood portion of the perfusate may range from about 5 mL to about 5000 mL, from about 50 mL to about 2500 mL, from about 100 mL to about 1000 mL, from about 150 mL to about 500 mL, about 50 mL, about 75 mL, about 100 mL, about 125 mL, about 150 mL, about 175 mL, about 200 mL, about 225 mL, about 250 mL, about 275 mL, about 300 mL, about 325 mL, about 350 mL, about 375 mL, about 400 mL, about 425 mL, about 450 mL, about 475 mL, about 500 mL, about 550 mL, about 600 mL, about 650 mL, about 700 mL, about 750 mL, about 800 mL, about 850 mL, about 900 mL, about 950 mL, or about 1000 mL.

The ratio of autologous blood to blood matched from donors in the blood that is circulated through the closed circuit may be adjusted, as needed, to obtain a blood mixture that would be most receptive to the drug and would generate the least immune response upon introduction of the drug. In some embodiments the ratio may range from about 1:100 to about 100:1, from about 1:80 to about 80:1, from about 1:50 to about 50:1, from about 1:30 to about 30:1, from about 1:20 to about 20:1, from about 1:10 to about 10:1, from about 1:8 to about 8:1, from about 1:5 to about 5:1, from about 1:3 to about 3:1, or from about 1:2 to about 2:1 of (volume autologous blood):(volume blood matched from donors).

The flow rate of the perfusate through the closed circuit may be adjusted to match the patient's blood flow rate. As appreciated by one of ordinary skill in the art, the blood flow rate varies from patient to patient, and for any given patient, varies throughout the day. Accordingly, the flow rate of the perfusate circulated through the closed circuit may be adjusted in situ. The flow rate may be measured over the closed circuit. In at least one embodiment, the flow rate may be measured with a transonic probe (such as a clamp over tubing). In some embodiments, the flow rate of the perfusate, at any given time during the perfusion, may be within about 20%, within about 15%, within about 10%, within about 8%, within about 5%, within about 3%, within about 2%, within about 1%, or within about 0.5% of the patient's blood flow rate, based on mL/min units. It is important that the flow rate of the perfusate circulated through the closed circuit does not deviate significantly from the patient's own blood flow rate in order to avoid ischemia and/or under perfusion.

Exemplary flow rates for the perfusate circulated through the closed circuit may range, without limitations, from about 75 mL/min to about 750 mL/min, from about 100 mL/min to about 650 mL/min, from about 125 mL/min to about 600 mL/min, from about 150 mL/min to about 500 mL/min, from about 175 mL/min to about 400 mL/min, from about 200 mL/min to about 300 mL/min, about 150 mL/min, about 175 mL/min, about 200 mL/min, about 225 mL/min, about 250 mL/min, about 275 mL/min, about 300 mL/min, about 325 mL/min, or about 350 mL/min. In some embodiments, the system maintains a flow rate of the perfusate in the closed circuit at about 500 mL/min/1.73 m² of body surface area per kidney to about 650 mL/min/1.73 m² of body surface area per kidney for about 15 min to about 4 hours.

The perfusate may be circulated through the closed circuit for a duration ranging, without limitations, from about 5 minutes to about 5 hours, from about 15 minutes to about 4 hours, from about 30 minutes to about 3 hours, or from about 1 hour to about 2 hours. In some embodiments, the treatment duration may occur over the span of days, e.g., 1 day, 2 days, 3 days, 4 days, 5 days, 6 days, 7 days, and so on.

With the system disclosed herein, in some embodiments, a higher dose of drug than could otherwise be administered safely through systemic delivery may be administered directly and only to the kidney or kidneys. In some embodiments, a lower overall dose of drug may be required to attain the same therapeutic effect (as was attained with a larger dose that was subjected to systemic circulation or that was subjected to only partial isolation of the renal circulation), since there may be substantially no leakage of the perfusate outside of the kidney or kidneys.

In some embodiments, less than about 50% v/v, less than about 40% v/v, less than about 30% v/v, less than about 20% v/v, less than about 15% v/v, less than about 10% v/v, less than about 5% v/v, less than about 4% v/v, less than about 3% v/v, less than about 2% v/v, less than about 1% v/v, less than about 0.5% v/v, or substantially no (0% v/v) perfusate (e.g., blood and/or drug) circulated through the closed circuit leaks outside of the closed circuit during the perfusion process.

The reduced perfusate leakage outside of the closed circuit (as compared to other methods disclosed in the art) may be due to the tight seal formed within the closed circuit and each individual component utilized in the closed circuit.

In at least one embodiment, some perfusate leakage from the closed circuit may remain. For instance, up to about 0.5% v/v, about 1% v/v, about 2% v/v, about 3% v/v, about 4% v/v, about 5% v/v, about 10% v/v, about 15% v/v, about 20% v/v, about 30% v/v, about 40% v/v, or about 50% v/v of the perfusate circulated through the closed circuit may leak outside of the closed circuit. Any drug amount lost through leakage of the perfusate may be replaced in the perfusate in order to keep the drug exposure to the kidney constant over the calculated exposure time. The calculated exposure time may, in at least one embodiment, range from about 5 minutes to about 5 hours, from about 15 minutes to about 4 hours, from about 30 minutes to about 3 hours, from about 1 hour to about 2 hours, or any sub-range in between.

### Therapeutic Compositions

Drugs suitable for treatment of the renal condition (i.e., drugs included in the perfusate) may include therapeutic polynucleotide sequences. In some embodiments, the therapeutic polynucleotide sequences may encode a protein for the treatment of a renal condition. The protein for treatment of the renal condition may be of human origin or may be derived from different species (e.g., without limitations, mouse, cat, pig or monkey). In some embodiments, the protein encoded by the therapeutic polynucleotide sequence may correspond to a gene expressed in a human kidney. Exemplary proteins and their mechanisms of action are described below.

Polycystin-1 (PC1) is the product of the PKD1 gene, the most common gene mutated in ADPKD. PC1 is a 4,303-amino acid glycoprotein composed of multiple domains; a large amino-terminal (N-terminal) extracellular region containing multiple protein-protein interaction motifs, an 11-transmembrane (TM) domain, and an approximately 200-amino acid carboxy-terminal (C-terminal) cytoplasmic tail capable of activating multiple signaling pathways. The N-terminal extracellular region is separated from the 11-TM domain by the G protein-coupled receptor (GPCR) autoproteolysis-inducing (GAIN) domain containing a GPCR Proteolytic Site (GPS). Cis-autoproteolytic cleavage occurs at the GPS, approximately 20 amino acids before the first TM domain, yielding an approximately 370-kDa N-terminal fragment (NTF) and an approximately 150-kDa C-terminal fragment (CTF). SEQ ID NO: 1 is the full amino acid sequence for PC1. SEQ ID NO: 2 is a nucleic acid sequence encoding PC1. SEQ ID NO: 3 is the amino acid sequence for the CTF. SEQ ID NO: 4 is a nucleic acid sequence encoding the CTF.

Expression of polycystin-2 (PC2), discussed in greater detail below, is necessary for the cis-autoproteolytic cleavage to occur. Further, mutations in PC1 that prevent cleavage at the GPS prevent proper maturation of PC1 and result in a protein incapable of exiting the endoplasmic reticulum. Upon cleavage at the GPS, the NTF and CTF remain non-covalently associated, until dissociated by ligand binding to the NTF. PC1 undergoes an additional cleavage event in the CTF that releases the ~200 amino acid C-terminal cytoplasmic tail (CTT). SEQ ID NO: 5 is the amino acid sequence for the CTT. SEQ ID NO: 6 is a nucleic acid sequence encoding the CTT.

PC1 is expressed in the epithelial cells of renal tubules, as well as in various other somatic tissues including liver, heart, bone, and endocrine glands. Within epithelial cells, PC1 is found in the cilium as well as in the lateral domain of the plasma membrane and adhesion complexes of polarized epithelial cells. Additionally, PC1 may shed from the apical or ciliary membranes in urinary exosome vesicles.

As a large, complex protein, PC1 has multiple functions. The extracellular N-terminal domain contains multiple PKD repeat motifs, leucine-rich repeat motifs, and a C-type lectin domain, each of which play established roles in protein-protein and protein-matrix interactions. The motifs present within the N-terminal domain in conjunction with the subcellular localization of PC1 support a role for PC1 in cell-cell and cell-matrix interactions. Additionally, PC1 may also participate in sensing fluid flow and pressure in the kidney.

PC1 possesses structural features consistent with being a member of the family of atypical adhesion G-protein coupled receptors (aGPCR) (Maser and Calvet, Cellular Signalling, 2020, Vol. 72), including the presence of the GAIN domain, which includes a GPS, a large N-terminal extracellular domain, a potential tethered agonist stalk peptide exposed upon GPS cleavage and NTF dissociation, and the presence of a G-protein binding domain within the intracellular C-terminal region. Cleavage of PC1 at the GPS generates the CTF of PC1, which remains non-covalently bound to the NTF. In the context of the CTF functioning as an aGPCR, cleavage at the GPS is not sufficient to activate GPCR signaling. Rather, binding of a ligand to the NTF, potentially a Wnt ligand (reviewed in Padovano et. al., Cellular Signalling, 2020, Vol. 72), displaces the NTF, exposing the stalk peptide, which in turn serves as a tethered ligand, inserting into the extracellular loops present within the CTF and thus stimulating G protein-mediated signaling. G protein α-subunits activated by PC1 positively regulate the activity of c-Jun N-terminal kinase (JNK) and the AP-1 transcription factor which controls differentiation, apoptosis, and cell proliferation. Dysregulation of these functions is consistent with the hallmarks of cyst formation in ADPKD, which include loss of epithelial cell polarization and uncontrolled cell proliferation. Importantly, mutations within PC1 that prevent GPS cleavage or mutations that inhibit the G-protein binding by the CTF lead to cyst formation and progression of ADPKD (Parnell et. al., Human Molecular Genetics, 2018, Vol. 27; Zhang et. al., Development, 2018, Vol. 145). Thus, a gene therapeutic that provides for the production of the CTF of PC1 is expected to restore PC1-mediated GPCR signaling and thus prevent, delay, or reverse the formation of cysts, and may provide an effective treatment for ADPKD caused by mutations in PKD1. This function of the CTF of PC1 may be independent of or antagonized by PC2. Further, as a role of PC2 in PC1 function is to promote cleavage of PC1 at the GPS, providing a gene therapeutic that produces the CTF independent of PC1 autoproteolysis may also overcome loss or reduction in function of PC2, so could be used to effectively treat ADPKD caused by mutations in the PKD1 or PKD2 genes.

The CTT of PC1 is an approximate 200 amino acid subdomain of the CTF generated by cleavage of intact PC1 or the CTF via an as yet unidentified proteolytic mechanism. The CTT is implicated in a number of signaling pathways and appears to contain both a nuclear localization sequence and mitochondrial targeting sequence, and is capable of localizing to both the nucleus and the mitochondria (reviewed in Padovano et al., Cellular Signalling, 2020, Vol. 72). The CTT may translocate to the nucleus with components of the Wnt pathway, STAT6/p100 and other STAT family members and regulate transcriptional pathways that control cell proliferation and apoptosis. This translocation to the nucleus may be part of the mechanosensing function of PC1. The CTT may also translocate to mitochondria. In Drosophila, translocation of the CTT to mitochondria leads to a reduced capacity for endurance exercise and an increase in CO₂ production, demonstrating that the CTT may regulate mitochondrial function. Given the role of dysregulation of mitochondrial function and metabolism in ADPKD, regulation of mitochondrial function by the CTT may play a critical role in renal epithelial homeostasis, and loss of this function may play a critical role in cyst formation and growth in ADPKD. In PKD1 knock-out mice, introduction of a gene coding for the CTT prevents or reduces disease progression via a mechanism that may in part involve regulation of mitochondrial function (Onuchic et. al., Nature Communications, 2023, Vol. 14). Likewise, in zebra fish or Xenopus oocytes deficient in PC1, introduction of an mRNA coding for the CTT reduces cell proliferation and promotes apoptosis, and thus reverses cyst formation and other features associated with ADPKD (Zhang et. al. Development, 2018, Vol. 145; Merrick et. al., Developmental Cell 2012, Vol. 22). Thus, a gene therapeutic that provides for the production of the CTT of PC1 is expected to restore PC1-mediated functions and thus prevent, delay, or reverse the formation of cysts, and may provide an effective treatment for ADPKD caused by mutations in PKD1. This function of the CTT of PC1 may be independent of or antagonized by PC2. Further, as the presence of PC2 may stimulate cleavage of PC1 or the CTF resulting in CTT release, providing a gene therapeutics that produces the CTT independent of PC2 may also overcome loss or reduction in function of PC2, and are contemplated to be useful in effectively treating ADPKD caused by mutations in the PKD1 or PKD2 genes.

Polycystin-2 (PC2) is the protein product of the PKD2 gene, for which mutations within also cause ADPKD. PC2, also known as TRP2, is a 968 amino acid protein, which includes 6 trans-membrane spanning domains, and intracellular N- and C-termini. PC2 functions as a Ca²⁺ permeable nonselective cation channel, homologous to the transient receptor potential family of cation channels. A portion of the cellular pool colocalizes with PC1 to the cilium, whereas the majority of the cellular pool of PC2 appears to reside in intracellular compartments where it may modulate the release of Ca²⁺ from intracellular stores. The channel activity of the ciliary pool of the PC1/PC2 complex may respond to ciliary bending and serve to mediate the cilium's role in transducing mechanical or chemical stimuli necessary for renal function. PC2 is also required for appropriate processing and localization of PC1. Loss or reduction of PC2 leads to cyst formation and growth. In mice, expression of PC2 in a PC2 conditional knock-out mouse reverses the ADPKD (Dong et. al., Nature Genetics, 2021, Vol. 53). A gene therapeutic that provides for the production of PC2 is contemplated to restore PC2 function and thus prevent, delay, or reverse the formation of cysts, and may provide an effective treatment for ADPKD caused by mutations in PKD2. Restoration of PC2 function may involve providing Ca²⁺ modulating functions of PC2 or may involve enabling the processing and proper localization of PC1. SEQ ID NO: 7 is the full amino acid sequence for PC2. SEQ ID NO: 8 is a nucleic acid sequence encoding PC1.

Exemplary proteins for use in accordance with the embodiments described herein may include, without limitations, PC1 (SEQ ID NO: 1), PC2 (SEQ ID NO: 7), COL4A5, COL3A4, COL4A4, APOL1, NPHP1, MUC1, UMON, REN, HNF1B, CD2AP, MYO1E, CFH, CFI, CD46, C3, functional fragments thereof, functional subdomains thereof (e.g., SEQ ID NO: 3, SEQ ID NO: 5), functional variants thereof, or combinations thereof. The protein or proteins used may also be functional variants of the proteins mentioned herein and may exhibit a significant amino acid sequence identity compared to the original protein. For instance, the amino acid identity may amount to at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%. In this context, the term “functional variant” means that the variant of the protein is capable of, partially or completely, fulfilling the function of the naturally occurring corresponding protein. Functional variants of a protein may include, for example, proteins that differ from their naturally occurring counterparts by one or more amino acid substitutions, deletions, or additions.

The amino acid substitutions can be conservative or non-conservative. It is preferred that the substitutions are conservative substitutions, i.e., a substitution of an amino acid residue by an amino acid of similar polarity, which acts as a functional equivalent. Preferably, the amino acid residue used as a substitute is selected from the same group of amino acids as the amino acid residue to be substituted. For example, a hydrophobic residue can be substituted with another hydrophobic residue, or a polar residue can be substituted with another polar residue having the same charge. Functionally homologous amino acids, which may be used for a conservative substitution comprise, for example, non-polar amino acids such as glycine, valine, alanine, isoleucine, leucine, methionine, proline, phenylalanine, and tryptophan. Examples of uncharged polar amino acids comprise serine, threonine, glutamine, asparagine, tyrosine and cysteine. Examples of charged polar (basic) amino acids comprise histidine, arginine, and lysine. Examples of charged polar (acidic) amino acids comprise aspartic acid and glutamic acid.

Also considered as variants are proteins that differ from their naturally occurring counterparts by one or more (e.g., 2, 3, 4, 5, 10, or 15) additional amino acids. These additional amino acids may be present within the amino acid sequence of the original protein (i.e., as an insertion), or they may be added to one or both termini of the protein. Basically, insertions can take place at any position if the addition of amino acids does not impair the capability of the polypeptide to fulfill the function of the naturally occurring protein in the treated subject. Moreover, variants of proteins also comprise proteins in which, compared to the original polypeptide, one or more amino acids are lacking. Such deletions may affect any amino acid position provided that it does not impair the ability to fulfill the normal function of the protein.

Finally, variants of target proteins also refer to proteins that differ from the naturally occurring protein by structural modifications, such as modified amino acids. Modified amino acids are amino acids which have been modified either by natural processes, such as processing or post-translational modifications, or by chemical modification processes known in the art. Typical amino acid modifications comprise phosphorylation, glycosylation, acetylation, O-linked N-acetylglucosamination, glutathionylation, acylation, branching, ADP ribosylation, crosslinking, disulfide bridge formation, formylation, hydroxylation, carboxylation, methylation, demethylation, amidation, cyclization, and/or covalent or non-covalent bonding to phosphotidylinositol, flavine derivatives, lipoteichonic acids, fatty acids, or lipids.

The therapeutic polynucleotide sequence encoding the target protein may be administered to the subject to be treated in the form of a gene therapy vector, i.e., a nucleic acid construct which comprises the coding sequence, including the translation and termination codons, next to other sequences required for providing expression of the exogenous nucleic acid such as promoters, kozak sequences, polyA signals, and the like.

For example, the gene therapy vector may be part of a mammalian expression system. Useful mammalian expression systems and expression constructs are commercially available. Also, several mammalian expression systems are distributed by different manufacturers and can be employed in the present invention, such as plasmid- or viral vector based systems, e.g., LENTI-Smart™ (InvivoGen), GenScript™ Expression vectors, pAdVAntage™ (Promega), ViraPower™ Lentiviral, Adenoviral Expression Systems (Invitrogen), and adeno-associated viral expression systems (Cell Biolabs).

Gene therapy vectors for expressing an exogenous therapeutic polynucleotide sequence of the invention can be, for example, a viral or non-viral expression vector, which is suitable for introducing the exogenous therapeutic polynucleotide sequence into a cell for subsequent expression of the protein encoded by said nucleic acid. The expression vector can be an episomal vector, i.e., one that is capable of self-replicating autonomously within the host cell, or an integrating vector, i.e., one which stably incorporates into the genome of the cell. The expression in the host cell can be constitutive or regulated (e.g., inducible).

In a certain embodiment, the gene therapy vector is a viral expression vector. Viral vectors for use in the present invention may comprise a viral genome in which a portion of the native sequence has been deleted in order to introduce a heterogeneous polynucleotide without destroying the infectivity of the virus. Due to the specific interaction between virus components and host cell receptors, viral vectors are highly suitable for efficient transfer of genes into target cells. Suitable viral vectors for facilitating gene transfer into a mammalian cell can be derived from different types of viruses, for example, from an AAV, an adenovirus, a retrovirus, a herpes simplex virus, a bovine papilloma virus, a lentivirus, a vaccinia virus, a polyoma virus, a sendai virus, orthomyxovirus, paramyxovirus, papovavirus, picornavirus, pox virus, alphavirus, or any other viral shuttle suitable for gene therapy, variations thereof, and combinations thereof.

“Adenovirus expression vector” or “adenovirus” is meant to include those constructs containing adenovirus sequences sufficient (a) to support packaging of the therapeutic polynucleotide sequence construct, and/or (b) to ultimately express a tissue and/or cell-specific construct that has been cloned therein. In one embodiment of the invention, the expression vector comprises a genetically engineered form of adenovirus. Knowledge of the genetic organization of adenovirus, a 36 kilobase (kb), linear, double-stranded DNA virus, allows substitution of large pieces of adenoviral DNA with foreign sequences up to 7 kb.

Adenovirus growth and manipulation is known to those of skill in the art, and exhibits broad host range in vitro and in vivo. This group of viruses can be obtained in high titers, e.g., 10⁹ to 10¹¹ plaque-forming units per mL, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and/or therapeutic potential as in vivo gene transfer vectors.

Retroviruses (also referred to as “retroviral vector”) may be chosen as gene delivery vectors due to their ability to integrate their genes into the host genome, transferring a large amount of foreign genetic material, infecting a broad spectrum of species and cell types and for being packaged in special cell-lines.

The retroviral genome contains three genes, gag, pol, and env, that encode for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest (GOI) is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

The retrovirus can be derived from any of the subfamilies. For example, vectors from Murine Sarcoma Virus, Bovine Leukemia, Virus Rous Sarcoma Virus, Murine Leukemia Virus, Mink-Cell Focus-Inducing Virus, Reticuloendotheliosis Virus, or Avian Leukosis Virus can be used. The skilled person will be able to combine portions derived from different retroviruses, such as LTRs, tRNA binding sites, and packaging signals to provide a recombinant retrovirus. These retroviruses are then normally used for producing transduction competent retroviral vector particles. For this purpose, the vectors are introduced into suitable packaging cell lines. Retroviruses can also be constructed for site-specific integration into the DNA of the host cell by incorporating a chimeric integrase enzyme into the retroviral particle.

Because herpes simplex virus (HSV) is neurotropic, it has generated considerable interest in treating nervous system disorders. Moreover, the ability of HSV to establish latent infections in non-dividing neuronal cells without integrating into the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient multiplicity of infection (MOI) and in a lessened need for repeat dosing. Avirulent variants of HSV have been developed and are readily available for use in gene therapy contexts.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu, and nef are deleted making the vector biologically safe.

Lentiviral vectors are plasmid-based or virus-based, and are configured to carry the essential sequences for incorporating foreign nucleic acid, for selection and for transfer of the nucleic acid into a host cell. The gag, pol, and env genes of the vectors of interest also are known in the art. Thus, the relevant genes are cloned into the selected vector and then used to transform the target cell of interest.

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome. Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus results in a level of expression that is higher than that of conventional vectors, with the transgenes accumulating at 10% or more of the infected cell's protein in 24 hours.

The empty capsids of papovaviruses, such as the mouse polyoma virus, have received attention as possible vectors for gene transfer. The use of empty polyoma was first described when polyoma DNA and purified empty capsids were incubated in a cell-free system. The DNA of the new particle was protected from the action of pancreatic DNase. The reconstituted particles were used for transferring a transforming polyoma DNA fragment to rat FIII cells. The empty capsids and reconstituted particles consist of all three of the polyoma capsid antigens VP1, VP2, and VP3.

AAVs are parvoviruses belonging to the genus Dependovirus. They are small, nonenveloped, single-stranded DNA viruses which require a helper virus in order to replicate. Co-infection with a helper virus (e.g., adenovirus, herpes virus, or vaccinia virus) is necessary in order to form functionally complete AAV virions. In vitro, in the absence of co-infection with a helper virus, AAV establishes a latent state in which the viral genome exists in an episomal form, but infectious virions are not produced. Subsequent infection by a helper virus “rescues” the genome, allowing it to be replicated and packaged into viral capsids, thereby reconstituting the infectious virion. Recent data indicate that in vivo both wild type AAV and recombinant AAV predominantly exist as large episomal concatemers. In one embodiment, the gene therapy vector used herein is an AAV vector. The AAV vector may be purified, replication incompetent, pseudotyped rAAV particles.

AAV are not associated with any known human diseases, are generally not considered pathogenic, and do not appear to alter the physiological properties of the host cell upon integration. AAV can infect a wide range of host cells, including non-dividing cells, and can infect cells from different species. In contrast to some vectors, which are quickly cleared or inactivated by both cellular and humoral responses, AAV vectors have been shown to induce persistent transgene expression in various tissues in vivo. The persistence of recombinant AAV-mediated transgenes in non-diving cells in vivo may be attributed to the lack of native AAV viral genes and the vector's ITR-linked ability to form episomal concatemers.

AAV is an attractive vector system for use in the cell transduction of the present invention as it has a high frequency of persistence as an episomal concatemer and it can infect non-dividing cells, including cardiomyocytes, thus making it useful for delivery of genes into mammalian cells, for example, in tissue culture and in vivo.

Typically, rAAV is made by cotransfecting a plasmid containing the GOI flanked by the two AAV terminal repeats and/or an expression plasmid containing the wild-type AAV coding sequences without the terminal repeats, for example pIM45. The cells are also infected and/or transfected with adenovirus and/or plasmids carrying the adenovirus genes required for AAV helper function. Stocks of rAAV made in such a fashion are contaminated with adenovirus, which must be physically separated from the rAAV particles (for example, by cesium chloride density centrifugation or column chromatography). Alternatively, adenovirus vectors containing the AAV coding regions and/or cell lines containing the AAV coding regions and/or some or all of the adenovirus helper genes could be used. Cell lines carrying the rAAV DNA as an integrated provirus can also be used.

Multiple serotypes of AAV exist in nature, with at least twelve serotypes (AAV1 through AAV13). Despite the high degree of homology, the different serotypes have tropisms for different tissues. Upon transfection, AAV elicits only a minor immune reaction (if any) in the host. Therefore, AAV is highly suited for gene therapy approaches.

The present disclosure may be directed in some embodiments to a drug comprising an AAV vector that is one or more of AAV1, AAV2, AAV3, AAV4, AAV5, AAV6, AAV7, AAV8, AAV9, AAV10, AAV11, AAV13, ANC AAV, chimeric AAV derived thereof, variations thereof, and combinations thereof, which will be even better suitable for high efficiency transduction in the tissue of interest. In at least one embodiment, the gene therapy vector is an AAV serotype 1 vector. In at least one embodiment, the gene therapy vector is an AAV serotype 2 vector. In at least one embodiment, the gene therapy vector is an AAV serotype 3 vector. In at least one embodiment, the gene therapy vector is an AAV serotype 4 vector. In at least one embodiment, the gene therapy vector is an AAV serotype 5 vector. In at least one embodiment, the gene therapy vector is an AAV serotype 6 vector. In at least one embodiment, the gene therapy vector is an AAV serotype 7 vector. In at least one embodiment, the gene therapy vector is an AAV serotype 8 vector. In at least one embodiment, the gene therapy vector is an AAV serotype 9 vector. In at least one embodiment, the gene therapy vector is an AAV serotype 10 vector. In at least one embodiment, the gene therapy vector is an AAV serotype 11 vector. In at least one embodiment, the gene therapy vector is an AAV serotype 12 vector.

A suitable dose of AAV for humans may be in the range of about 1×10⁸ vector genomes per kilogram of body weight (vg/kg) to about 3×10¹⁴ vg/kg, about 1×10⁸ vg/kg, about 1×10⁹ vg/kg, about 1×10¹⁰ vg/kg, about 1×10¹¹ vg/kg, about 1×10¹² vg/kg, about 1×10¹³ vg/kg, or about 1×10¹⁴ vg/kg. The total amount of viral particles or DRP is, is about, is at least, is at least about, is not more than, or is not more than about, 5×10¹⁵ vg/kg, 4×10¹⁵ vg/kg, 3×10¹⁵ vg/kg, 2×10¹⁵ vg/kg, 1×10¹⁵ vg/kg, 9×10¹⁴ vg/kg, 8×10¹⁴ vg/kg, 7×10¹⁴ vg/kg, 6×10¹⁴ vg/kg, 5×10¹⁴ vg/kg, 4×10¹⁴ vg/kg, 3×10¹⁴ vg/kg, 2×10¹⁴ vg/kg, 1×10¹⁴ vg/kg, 9×10¹³ vg/kg, 8×10¹³ vg/kg, 7×10¹³ vg/kg, 6×10¹³ vg/kg, 5×10¹³ vg/kg, 4×10¹³ vg/kg, 3×10¹³ vg/kg, 2×10¹³ vg/kg, 1×10¹³ vg/kg, 9×10¹² vg/kg, 8×10¹² vg/kg, 7×10¹² vg/kg, 6×10¹² vg/kg, 5×10¹² vg/kg, 4×10¹² vg/kg, 3×10¹² vg/kg, 2×10¹² vg/kg, 1×10¹² vg/kg, 9×10¹¹ vg/kg, 8×10¹¹ vg/kg, 7×10¹¹ vg/kg, 6×10¹¹ vg/kg, 5×10¹¹ vg/kg, 4×10¹¹ vg/kg, 3×10¹¹ vg/kg, 2×10¹¹ vg/kg, 1×10¹¹ vg/kg, 9×10¹⁰ vg/kg, 8×10¹⁰ vg/kg, 7×10¹⁰ vg/kg, 6×10¹⁰ vg/kg, 5×10¹⁰ vg/kg, 4×10¹⁰ vg/kg, 3×10¹⁰ vg/kg, 2×10¹⁰ vg/kg, 1×10¹⁰ vg/kg, 9×10⁹ vg/kg, 8×10⁹ vg/kg, 7×10⁹ vg/kg, 6×10⁹ vg/kg, 5×10⁹ vg/kg, 4×10⁹ vg/kg, 3×10⁹ vg/kg, 2×10⁹ vg/kg, 1×10⁹ vg/kg, 9×10⁸ vg/kg, 8×10⁸ vg/kg, 7×10⁸ vg/kg, 6×10⁸ vg/kg, 5×10⁸ vg/kg, 4×10⁸ vg/kg, 3×10⁸ vg/kg, 2×10⁸ vg/kg, or 1×10⁸ vg/kg, or falls within a range defined by any two of these values. The above listed dosages being in vg/kg renal tissue units.

With the systems and methods disclosed herein, in some embodiments, a higher dose of drug than could otherwise be administered safely through systemic delivery may be administered directly and only to the kidney, since there is substantially no leakage of the perfusate outside of the kidney. Without being construed as limiting, it is believed that AAV toxicity may be due to systemic effects such as hepatotoxicity, platelet activation and loss, and complement activation and loss. All of these toxicities and others may be reduced, minimized, or completely avoided via the loco-regional perfusate application described in the methods and systems disclosed herein. As such, doses up to about 1×10¹⁵ vg/kg renal tissue or greater may be well tolerated. In at least one embodiment, AAV doses to the kidney, expressed as vg/kg renal tissue, may exceed the highest systemically administered doses by a factor of about 2 to about 200, about 5 to about 150, about 10 to about 100, or any sub-range therein.

Apart from viral vectors, non-viral expression constructs may also be used for introducing a gene encoding a target protein or a functioning variant or fragment thereof into a cell of a patient. Non-viral expression vectors which permit the in vivo expression of protein in the target cell include, for example, a plasmid, a modified RNA, an mRNA, a cDNA, antisense oligomers, DNA-lipid complexes, nanoparticles, exosomes, any other non-viral shuttle suitable for gene therapy, variations thereof, and a combination thereof.

Apart from viral vectors and non-viral expression vectors, nuclease systems may also be used, in conjunction with a vector and/or an electroporation system, to enter into a cell of a patient and introduce therein a gene encoding a target protein or a functioning variant or fragment thereof. Exemplary nuclease systems may include, without limitations, a clustered regularly interspaced short palindromic repeats (CRISPR), a DNA cutting enzyme (e.g., Cas9), meganucleases, TALENs, zinc finger nucleases, any other nuclease system suitable for gene therapy, variations thereof, and a combination thereof. For instance, in one embodiment, one viral vector (e.g., AAV) may be used for a nuclease (e.g., CRISPR) and another viral vector (e.g., AAV) may be used for a DNA cutting enzyme (e.g., Cas9) to introduce both (the nuclease and the DNA cutting enzyme) into a target cell.

Other vector delivery systems which can be employed to deliver a therapeutic polynucleotide sequence encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific. Receptor-mediated gene targeting vehicles may include two components: a cell receptor-specific ligand and a DNA-binding agent.

Suitable methods for the transfer of non-viral vectors into target cells are, for example, the lipofection method, the calcium-phosphate co-precipitation method, the DEAE-dextran method and direct DNA introduction methods using micro-glass tubes, ultrasound, electroporation, and the like. Prior to the introduction of the vector, the renal cells may be treated with a permeabilization agent, such as phosphatidylcholine, streptolysins, sodium caprate, decanoylcarnitine, tartaric acid, lysolecithin, Triton X-100, and the like. Exosomes may also be used to transfer naked DNA or AAV-encapsidated DNA.

A gene therapy vector of the invention may comprise a promoter that is functionally linked to the nucleic acid sequence encoding to the target protein. The promoter sequence should be compact and ensure a strong expression. Preferably, the promoter provides for an expression of the target protein in the kidney of the patient that has been treated with the gene therapy vector. In some embodiment, the gene therapy vector comprises a nephron-specific promoter which is operably linked to the nucleic acid sequence encoding the target protein. As used herein, a “nephron-specific promoter” refers to a promoter whose activity in renal cells is at least 2-fold higher than in any other non-renal cell type. Preferably, a nephron-specific promoter suitable for being used in the vector of the invention has an activity in renal cells which is at least 5-fold, at least 10-fold, at least 15-fold, at least 20-fold, at least 25-fold, or at least 50-fold higher compared to its activity in a non-renal cell type. Moreover, a nephron-specific promoter may be specific to a particular subunit of the nephron (e.g., proximal tubule, distal tubule, loop of Henle, collecting duct, glomerulus, etc.) to provide higher or exclusive expression in that particular subunit.

The nephron-specific promoter may be a selected human promoter, or a promoter comprising a functionally equivalent sequence having at least about 80%, at least about 90%, at least about 95%, at least about 96%, at least about 97%, at least about 98%, or at least about 99% sequence identity to the selected human promoter. Exemplary non-limiting promoters may include kidney-specific cadherin (KSPC), Na⁺/glucose co-transporter 2 (SGLT2), sodium potassium 2 chloride co-transporter 2 (NKCC2), and E-cadherin (ECAD), or podocyte-specific promoters such as the podocin promoter NPHS2.

The vectors useful in the present invention may have varying transduction efficiencies. As a result, the viral or non-viral vector transduces more than, equal to, or at least about 10%, about 20%, about 30%, about 40%, about 50%, about 55%, about 60%, about 65%, about 70%, about 75%, about 80%, about 85%, about 90%, about 95%, about 99%, or 100% of the cells of the targeted vascular territory. More than one vector (viral or non-viral, or combinations thereof) can be used simultaneously or in sequence. This can be used to transfer more than one polynucleotide, and/or target more than one type of cell. Where multiple vectors or multiple agents are used, more than one transduction/transfection efficiency can result.

Pharmaceutical compositions that contain gene therapy vectors may be prepared either as liquid solutions or suspensions. The pharmaceutical composition of the invention can include commonly used pharmaceutically acceptable excipients, such as diluents and carriers. In particular, the composition comprises a pharmaceutically acceptable carrier, e.g., water, saline, Ringer's solution, or dextrose solution. In addition to the carrier, the pharmaceutical composition may also contain emulsifying agents, pH buffering agents, stabilizers, dyes, and the like.

In at least one embodiment, a pharmaceutical composition will comprise a therapeutically effective gene dose, which is a dose that is capable of preventing or treating a renal condition in a subject, without being toxic to the subject. Prevention or treatment of the renal condition may be assessed as a change in a phenotypic characteristic associated with the renal condition with such change being effective to prevent or treat the renal condition. Thus, a therapeutically effective gene dose is typically one that, when administered in a physiologically tolerable composition, is sufficient to improve or prevent the pathogenic renal phenotype in the treated subject.

The following exemplary embodiments are now described:

Embodiment 1: A gene therapy vector adapted for transduction of renal cells of a human subject, the gene therapy vector comprising: an adeno-associated virus (AAV) vector; and a polynucleotide sequence packaged in the AAV vector, the polynucleotide sequence encoding a therapeutic protein having at least 80% sequence identity to a sequence selected from the group consisting of SEQ ID NO: 3, SEQ ID NO: 5, and SEQ ID NO: 7.

Embodiment 2: The gene therapy vector of Embodiment 1, wherein the polynucleotide sequence encodes for a therapeutic protein having at least 80% sequence identity to SEQ ID NO: 3.

Embodiment 3: The gene therapy vector of Embodiment 1, wherein the polynucleotide sequence encodes for a therapeutic protein having at least 80% sequence identity to SEQ ID NO: 5.

Embodiment 4: The gene therapy vector of Embodiment 1, wherein the polynucleotide sequence encodes for a therapeutic protein having at least 80% sequence identity to SEQ ID NO: 7.

Embodiment 5: The gene therapy vector of any of the preceding Embodiments, wherein the polynucleotide sequence further comprises a promoter sequence operatively linked to the polynucleotide sequence encoding for the therapeutic protein.

Embodiment 6: The gene therapy vector of Embodiment 5, wherein the promoter sequence is selected from the group consisting of: SEQ ID NO: 9, SEQ ID NO: 10, SEQ ID NO: 11, SEQ ID NO: 12, SEQ ID NO: 13, and SEQ ID NO: 14.

Embodiment 7: The gene therapy vector of Embodiment 5, wherein the promoter sequence is selected from the group consisting of: SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28.

Embodiment 8: The gene therapy vector of any of the preceding Embodiments, wherein a serotype of the AAV vector is selected from the group consisting of AAV1, AAV2, AAV3, AAV4, AAV5, AAV6, AAV7, AAV8, AAV9, AAV10, AAV11, AAV12, and AAV13.

Embodiment 9: The gene therapy vector of any of the preceding Embodiments, wherein a serotype of the AAV vector is AAV5.

Embodiment 10: A gene therapy drug comprising: the gene therapy vector of any of the preceding Embodiments; and a pharmaceutically acceptable carrier.

Embodiment 11: A method of treating a kidney-related disease comprising administering to a patient in need thereof a therapeutic dose of the gene therapy drug of Embodiment 10.

Embodiment 12: A method of performing gene replacement of a mutated gene comprising administering to a patient in need thereof a therapeutic dose of the gene therapy drug of Embodiment 10.

Embodiment 13: A method of treating autosomal dominant polycystic kidney disease (ADPKD) in a subject, the method comprising: administering to the subject a therapeutic dose of a drug comprising the gene therapy vector of any of Embodiments 1-9 and a pharmaceutically acceptable carrier.

Embodiment 14: A method of performing localized delivery of a polynucleotide sequence to renal cells in a kidney of a mammalian subject, the method comprising: positioning a perfusion catheter in the renal artery of the kidney; positioning a recovery catheter in the renal vein of the kidney, wherein the perfusion catheter and the recovery catheter together with a membrane oxygenation device form a closed perfusion circuit through the kidney; and causing a perfusate to flow through the closed circuit, wherein the perfusate comprises the gene therapy drug of Embodiment 10, and wherein the closed circuit substantially isolates perfusion through the kidney from the systemic circulation of the subject.

Embodiment 15: The method of Embodiment 14, wherein the renal cells comprise tubular cells.

Embodiment 16: The method of any of Embodiments 14-15, wherein a dose of the AAV vector is delivered via the closed circuit and maintained at a concentration of at least about 5×10⁹ of vector genome per milliliter (mL) of plasma) during perfusion, and wherein the vector present leaking into systemic circulation of the subject remains less than 5×10⁷ of vector genome per mL of plasma during perfusion, wherein the perfusion is maintained for a total of about 30 minutes to about 90 minutes.

Embodiment 17: The method of any of Embodiments 14-16, wherein positioning the perfusion catheter in the renal artery comprises positioning the perfusion catheter via the arteria femoralis.

Embodiment 19: The method of any of Embodiments 14-17, wherein positioning the recovery catheter in the renal vein comprises positioning the perfusion catheter via percutaneous access through the vena femoralis or via the jugular vein.

Embodiment 19: The method of any of Embodiments 14-18, wherein positioning the recovery catheter in the renal vein comprises positioning the perfusion catheter via non-percutaneous cut-down access.

Embodiment 20: The method of any of Embodiments 14-19, wherein causing the perfusate to flow through the closed circuit comprises: causing the perfusate to pass through the membrane oxygenation device prior to entering the renal artery via the perfusion catheter.

Embodiment 21: The method of any of Embodiments 14-20, further comprising: adding additional perfusate to the closed circuit or diluting the perfusate by about 5% to about 50% v/v of a saline solution to account for bladder excretion volume.

Embodiment 22: The method of any of Embodiments 14-21, wherein the closed circuit maintains a flow rate of the perfusate at about 500 mL/min/1.73 m² of body surface area per kidney to about 650 mL/min/1.73 m² of body surface area per kidney for about 15 min to about 4 hours.

Embodiment 23: The method of any of Embodiments 14-22, wherein the closed circuit maintains a flow rate of the perfusate at about 150 mL/min/1.73 m² of body surface area per kidney to about 700 mL/min/1.73 m² of body surface area per kidney for about 15 min to about 4 hours.

Embodiment 24: The method of any of Embodiments 14-23, further comprising applying negative pressure at the recovery catheter, wherein the negative pressure ranges from about −100 mmHg to 120 mmHg.

Embodiment 25: The method of any of Embodiments 14-24, wherein one or more of the perfusion catheter and the recovery catheter are introduced percutaneously or non-percutaneously.

Embodiment 26: The method of any of Embodiments 14-25, wherein less than about 20% v/v, less than about 15% v/v, less than about 10% v/v, less than about 5% v/v, less than about 4% v/v, less than about 3% v/v, less than about 2% v/v, less than about 1% v/v, less than about 0.5% v/v, or substantially no (0% v/v) perfusate circulated through the closed circuit leaks outside of the closed circuit.

Embodiment 27: The method of any of Embodiments 14-26, wherein one or more of the perfusion catheter or the recovery catheter is a balloon catheter.

Embodiment 28: A method of delivering a therapeutic composition to a subject in need thereof, the method comprising locally delivering the therapeutic composition to a kidney of the subject while substantially avoiding introduction of the therapeutic composition into the systemic circulation or other organs, the therapeutic composition comprising the gene therapy drug of Embodiment 10.

### ILLUSTRATIVE EXAMPLES

The following examples are set forth to assist in understanding the disclosure and should not, of course, be construed as specifically limiting the embodiments described and claimed herein. Such variations of the embodiments, including the substitution of all equivalents now known or later developed, which would be within the purview of those skilled in the art, and changes in formulation or minor changes in experimental design, are to be considered to fall within the scope of the embodiments incorporated herein.

The LRP system discussed below includes the following components: a percutaneous arterial catheter for occlusive antegrade perfusion of the renal artery (accessed via the femoral artery); a percutaneous venous catheter for occlusion of the renal vein and return of venous blood to the LRP system (accessed via the jugular vein); and an ECMO device with a reservoir and associated tubing to provide oxygen and remove carbon dioxide from the blood in the LRP system. The LRP procedure starts when the arteries are anterogradely perfused with oxygenated blood, while the returning de-oxygenated blood is collected from the venous system via the venous catheter. The blood is then collected in the reservoir, oxygenated, and anterogradely re-infused into the organ via the arterial catheters. Blood samples can be taken, or drugs can be introduced, via the reservoir during the entire procedure.

### Example 1: LRP Procedure

LRP was performed on pigs utilizing the LRP system 1800 illustrated in and described with respect to FIG. 18. Accessory devices that were used in these examples example are listed in Table 1, including their intended uses and the use in the LRP system in accordance with the embodiments of the disclosure.

TABLE 1
Devices used for LRP procedure

| Item | Brand |
|---|---|
| FlowGate² balloon guide catheter | Stryker |
| Fogarty catheter | Fogarty |
| Blood pump head | Medtronic |
| Femoral access sheath: 12-Fr, length 25 cm | Boston Scientific |
| Guidewire (=GW) V18 | Boston Scientific |
| Catheter Launcher 7-Fr AL1.0 | Medtronic |
| Standard J-tip GW (0.035 cm, 260 mm length) | Cordis |
| Rosen Heavy duty (0.035 cm, 180 mm length) | Cordis |
| Jugular access sheath Dryseal 24-Fr 33 cm | Gore |
| Endo-Vent catheter | Edwards |
| Lunderquist Extra Stiff Wire Guide 0.035, | Cook |
| 260 cm (REF: TSMG-35-260-LES | |
| D100 Oxygenator set | Dideco-Livanova |
| Bio-Medicus 550 Bio-Console (ECMO pump | Medtronic |
| console) | |
| Pressure Wire X | Saint Jude Medical/ |
| | Abbott |
| Quantien Measurement System | Saint Jude Medical/ |
| | Abbott |
| Custom catheter | — |

The custom catheter was used as a venous recovery catheter, and included the following dimensions: a crossing profile of 19 Fr (6.3 mm); an inner diameter of 12 Fr (4.0 mm); a usable length of 80 cm; a balloon diameter of 25 mm; and a tip length of 20 mm (similar to the exemplary custom catheter shown in and described with respect to FIGS. 1-3). The materials included: Pebax 63 supported by a strong stainless-steel braid as the shaft; compliant Chronoprene 25A as the balloon; and Pebax 35 loaded with BaSO₄ for radiopacity in the tip. The custom catheter was designed to support a suction flow rate of about 800 mL/min at −80 mmHg.

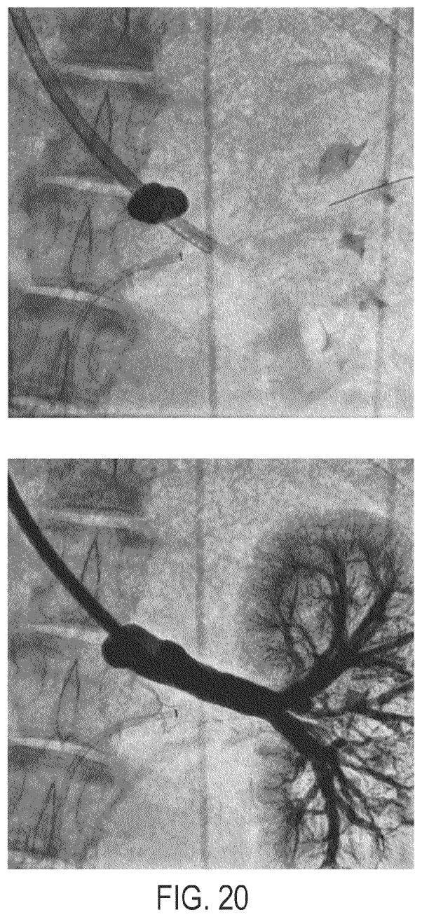

FIG. 20 includes radiographs showing successful placement of arterial and venous catheters in the renal artery and renal vein, respectively, of a porcine kidney. In the bottom image, a contrast agent is injected venously, revealing the kidney vasculature and the overally tightness of the closed system.

A detailed protocol of the LRP procedure that was followed in this example is now described:
(1) Place the study animal in dorsal recumbency;
(2) prepare the study animal for endovascular catheterization;
(3) By angiography, assess the angle of the kidney veins from both the jugular access and the groin access utilizing the least sharp angles;
(4) Access the arterial circulation of the kidney (side to be determined based on the angle) by a Stryker FlowGate² catheter from the femoral artery;
(5) Access the venous circulation (side and access point: to be determined based on the individual animal) using the custom venous catheter described above;
(6) Place catheters in their final location in an open configuration (i.e., balloon down) in order to inject some contrast fluid and to visualize kidney circulation;
(7) Place the catheters in the aorta and vena cava until procedure is to begin;
(8) Place the PressureWire X through the Flowgate² catheter and into one of the kidney arteries;
(9) Prepare the ECMO system by de-airing and priming with saline; venous and arterial lines are connected to the ECMO while clamped to avoid air being introduced;
(10) Turn on the ECMO pump;
(11) Unclamp the venous line;
(12) Begin exchanging saline for blood; if everything is stable, unclamp the arterial line and establish the LRP loop; suction on the venous side is variable and adapted to the needs (e.g., from −50 mmHg to 0);
(13) Place the venous catheter in position in the kidney vein;
(14) Inflate the balloon;
(15) Check the tightness and positioning of the catheters with a contrast injection;
(16) If the animal is stable:
   a. Seal the kidney artery with the Flowgate² catheter;
   b. Check: the tightness and position of the catheters with a contrast injection; the pressure in the kidney; the kidney versus systemic pressure ratio (targeting above 1); the reservoir volume; the RPM of the ECMO pump; and the flow of the catheters;
(17) If everything is stable for 5 minutes:
   a. Begin the glyceryl trinitrate infusion through the arterial line at a rate of 2 μg/kg body weight/min;
   b. Check: the pressure in the kidney; the kidney versus systemic pressure ratio (targeting above 1); the reservoir volume; the RPM of the ECMO pump; and the flow of the catheters;
(18) If everything is stable for 5 minutes:
   a. Begin treatment with gene therapy drug injected into the reservoir;
   b. For the first animal group (Group B1): administer a dose of 5.0E+13 vg (prepared by diluting 1.8 mL of the vector solution at a titer of 2.8E+13 vg/mL with 2.2 mL of vehicle);
   c. For the second animal group (Group B2): administer a dose of 6.0E+14 vg (equivalent to 21.4 mL of the vector solution at a titer of 2.8E+13 vg/mL);
(19) Continue the kidney LRP for 60 minutes;
(20) Every 5 minutes, check: the pressure in the kidney; the kidney versus systemic pressure ratio (targeting above 1); the reservoir volume; the RPM of the ECMO pump; the flow of the catheters; all hemodynamic and cardiovascular parameters (pressure, HR);
(21) Check urine output at t=0, 15, 30, 45 and 60 min after the start of the procedure;
(22) Pay attention to the LRP reservoir volume, as there may be overfilling due to the phrenic, gonadal, and adrenal veins, or loss of volume due to urine production; these deviations in volume can be managed dynamically;
(23) At t=0, 5, 15, 30, 45 and 60 min: collect blood samples:
   a. from peripheral blood for shedding analyses;
   b. from LRP system for vector infectivity analyses; and
   c. from LRP system for shedding analyses;
(24) At the end of the 60 minutes of kidney LRP:
   a. discontinue the glyceryl trinitrate;
   b. deflate the balloons; and
   c. disengage the catheters;
(25) Discard the entire LRP circuit, reservoir, blood pump, and catheters in an appropriate biosafety bin;
(26) Perform immediate post-surgical care, including but not limited to compression and administration of protamine;
The above procedure demonstrated that LRP of the kidney with a sealed closed-circuit was possible for at least 60 minutes. No acute sequalae was observed, and indigocarmine tests immediately after the LRP procedure showed that kidney function was normal/unaffected by the procedure.

Factors that may be useful in optimizing the LRP procedure to reduce leakage of perfusate into the systemic circulation and to avoid transduction of proximal and distal organs include, but are not limited to: perfusion time, drug dose, the AAV serotype used, the presence of neutralizing factors, endothelial permeability, flow rate, and perfusion pressure.

### Example 2: Biodistribution Studies

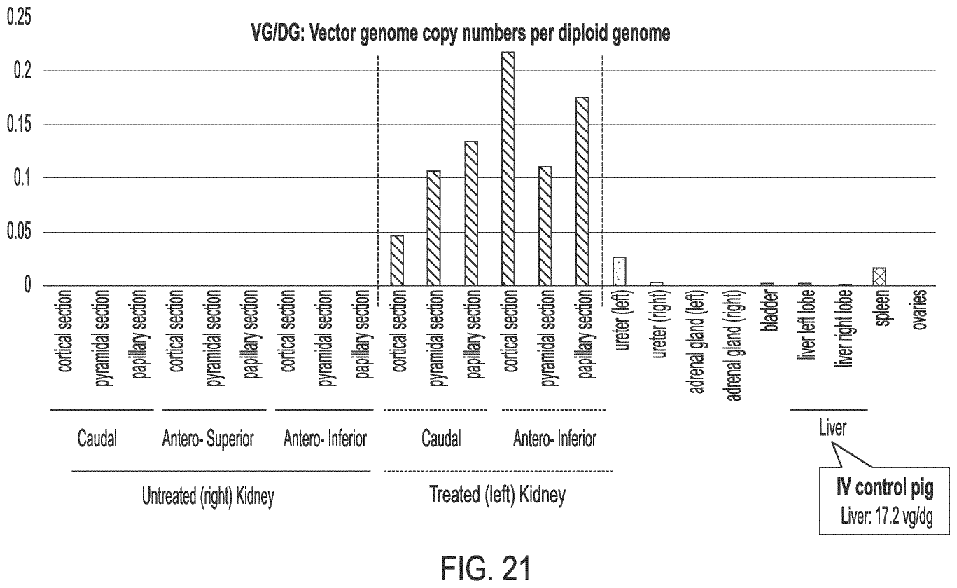

FIG. 21 is a plot showing kidney transduction and biodistribution of 0.05-0.25 vg/dg (vector genome copy numbers per diploid genome) after 60 min of LRP with the higher dose of 6.2E+14 vg/kg. No significant contamination of the untreated kidney, the liver, or other organs was detected, which demonstrated the tightness of the LRP closed-circuit.

An intravenous control animal was also tested. It was found that kidney LRP led to a more even transduction profile across the different sections measured, while the IV control showed preferential transduction in the cortical section of the kidney. Transduction in the liver was significantly less for kidney LRP versus the IV control, where 17.2 vg/dg was detected in the liver for the IV control while virtually no transduction was observable in the liver for kidney LRP.

### Example 3: Vector Quantification

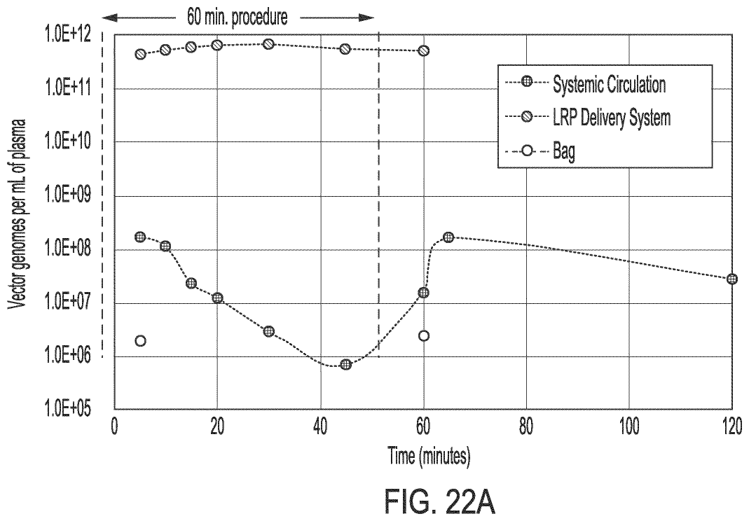

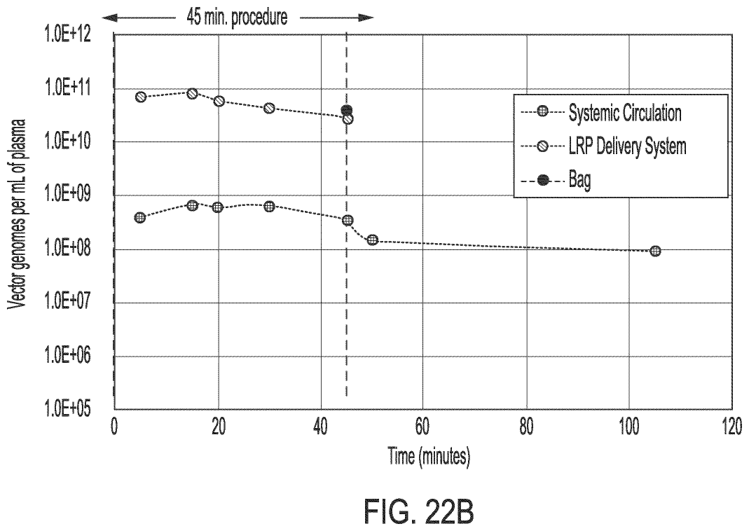

FIGS. 22A and 22B show vector genome per mL of plasma measured at various time points during kidney LRP high dose (6.2×10¹⁴ vg/kg, FIG. 22A) and low dose (5.6×10¹³ vg/kg, FIG. 22B). The results revealed high retention (low vector shedding) within the LRP circuit for 60 minutes, low exposure of the vector to the systemic circulation, and very low leakage of the vector into the urine (FIG. 22A). Exposure of the vector to the kidney appears to be maximized throughout the procedure.

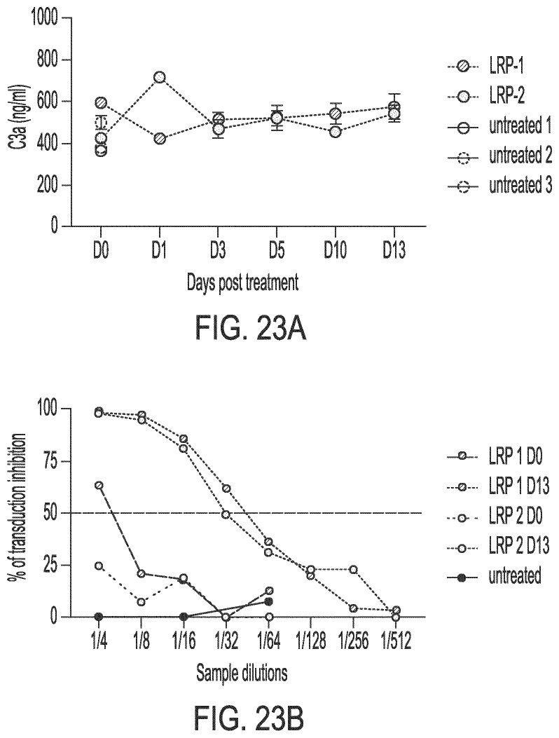

FIG. 23A is a plot of C3a levels for several days post kidney LRP treatment for two different animals (LRP-1 and LRP2). FIG. 23B is a plot of % transduction inhibition for various sample dilutions. Both reveal that anti-AAV neutralizing factors remained low for both animals, and that there was no complement activation following kidney LRP.

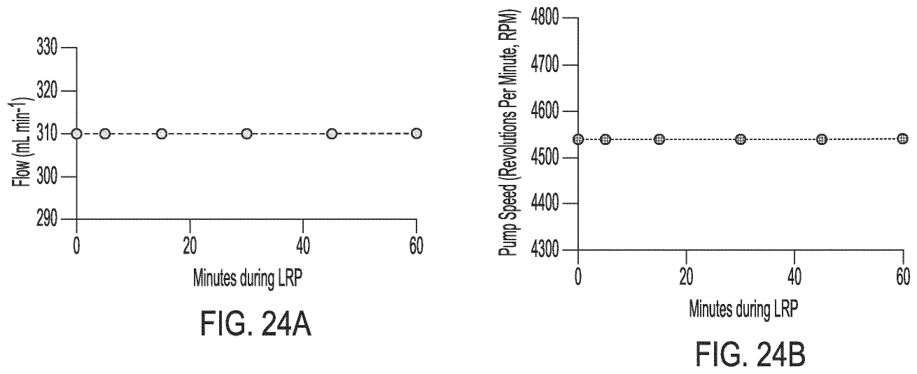

FIGS. 24A and 24B are plots of flow rate and pump speed, respectively, during kidney LRP, revealing a substantially constant flow rate of about 310 mL/min throughout the procedure.

These examples demonstrate targeted AAV delivery to the kidney, resulting in homogenous transgene biodistribution using a clinically relevant animal model. The embodiments described and exemplified herein enable the development of next generation advanced therapies for the kidney by minimizing systemic adverse effects, significantly reducing required vector doses, overcoming immunologic limitations, and with the potential to repeat treatment. Use of the LRP system and methods are contemplated for use with other therapeutic agents and strategies.

In LRP experiments of the kidney for GFP expression, GFP been found localized to kidney glomeruli, specifically in podocytes.

It is contemplated that the methodologies described herein may be applied to other organs as well by applying the LRP procedure to those organs. Exemplary organs may include, but are not limited to, the heart, the liver, or the pancreas. LRP of the heart is discussed in International Application No. PCT/IB2020/000692, filed Aug. 26, 2020, the disclosure of which is hereby incorporated by reference herein in its entirety.

### Example 4: Serotype Screening

Screening of serotypes to identify highly efficient serotypes for use in localized gene delivery to a kidney is now described as follows. A number of AAV vector candidates are first identified. In this study, AAV1, AAV2, AAV5, AAV6, and AAV9 serotypes were selected as the candidates. AAV vectors of each serotype were produced, each comprising a CMV-GFP transgene cassette. A perfusate was prepared containing each of the five AAV types for a total of 4.9×10¹⁴ vg (with 5.9×10¹³ vg per serotype). LRP was performed on a porcine kidney using similar protocols as described above and using custom catheters as described above. A total amount of vector injected into the closed loop was 6.4×10¹² vg/kg, and perfusion in the closed circuit was maintained for 60 minutes.

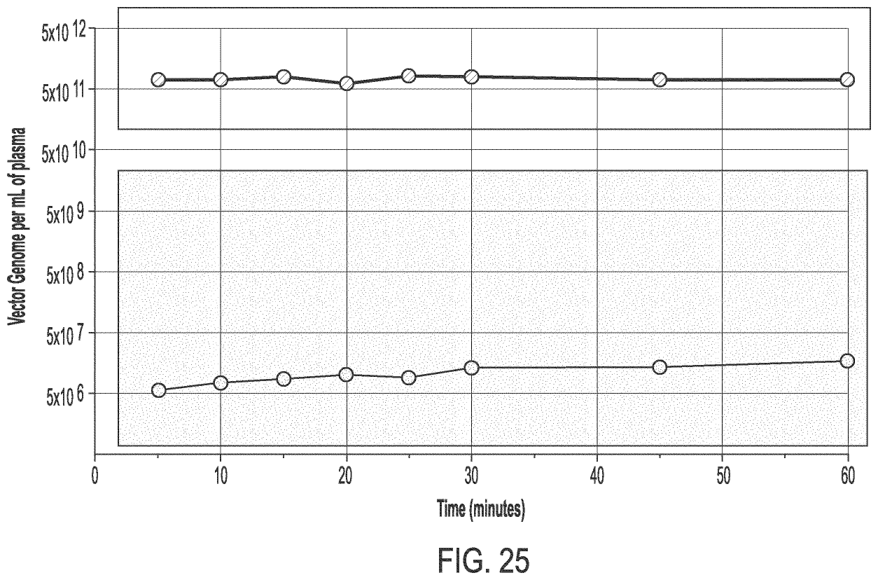

FIG. 25 is a plot showing vector genome per mL of plasma measured at various time points during the 60-minute kidney LRP procedure in the LRP closed circuit versus systemic circulation for the perfusate comprising the five different serotypes. The results revealed high retention (low vector shedding) within the LRP circuit for 60 minutes, and low exposure of the vector to the systemic circulation (less than 5×10⁷ vg per mL of plasma).

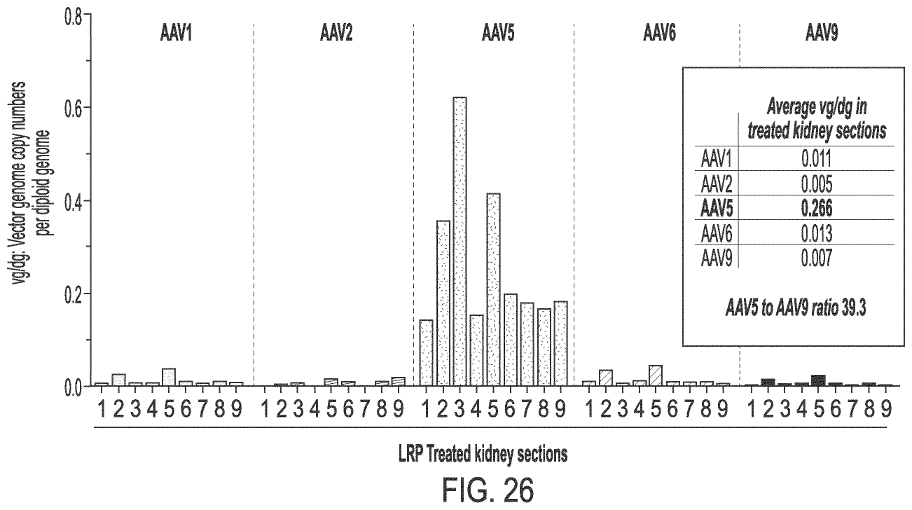

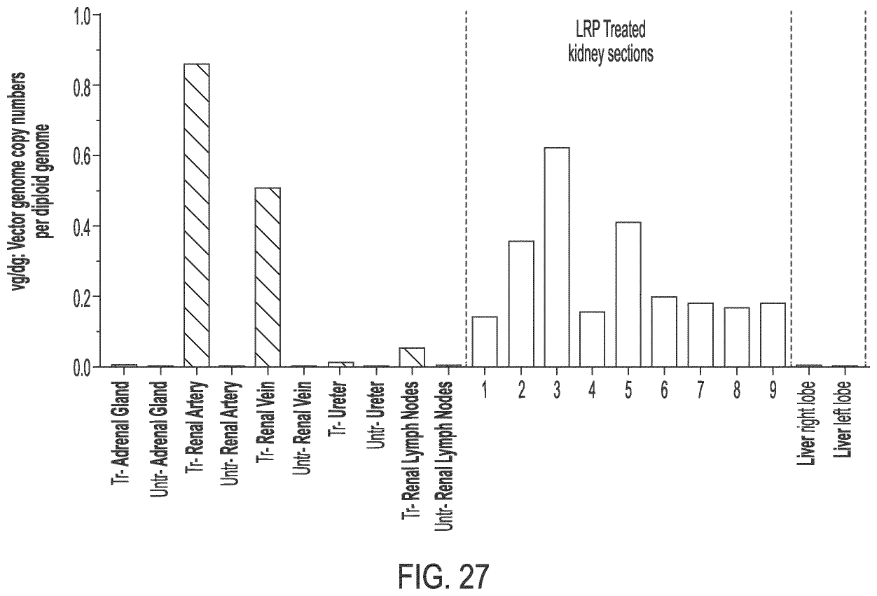

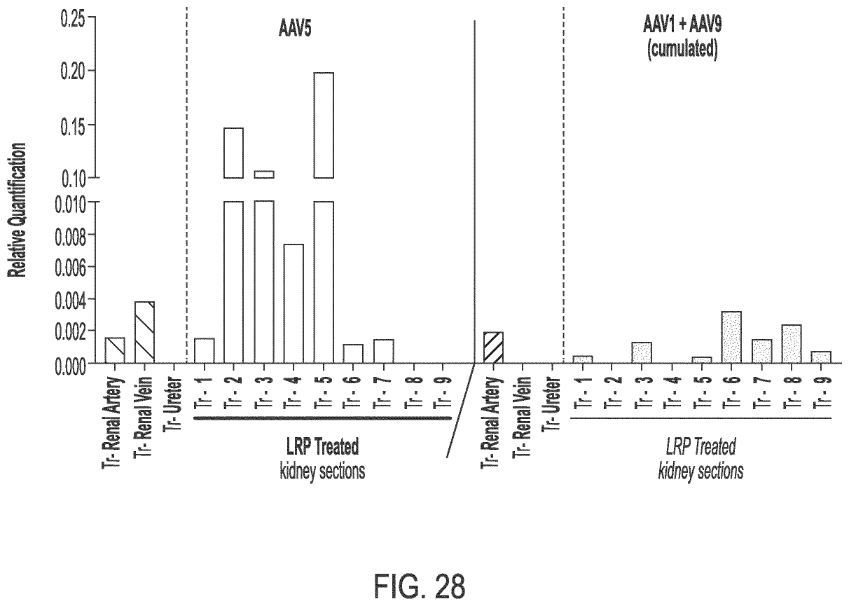

FIG. 26 is a plot showing biodistribution of the multiple AAV serotypes in the kidney based on measured vector genome copy numbers per diploid genome in various sections of the LRP-treated kidney, revealing AAV5 to have significantly greater efficiency than the other serotypes (39.3 times greater efficiency than AAV9). FIG. 28 is a plot showing relative quantification of transgene mRNA levels in the treated kidney sections for AAV5 versus cumulative AAV in the kidney. FIG. 27 is a plot showing biodistribution in the kidney compared to the liver and additional renal-associated tissues after perfusion for 60 minutes with the multiple AAV serotypes, revealing that transduction was limited to the treated kidney sections and renal arteries and veins, with minimal transduction occurring in the liver or the untreated kidney. Without wishing to be bound by theory, it is believed that the high efficiency of AAV5 can be attributed to a synergistic effect resulting from maintaining a high concentration of AAV5 in the closed circuit for a fixed time period (30 minutes to 1 hour) to allow for transduction. Without localization, systemic circulation is believed to result in a low amount of AAV5 transduction of the kidney, with a significant fraction of the AAV5 ending up in the liver or other organs.

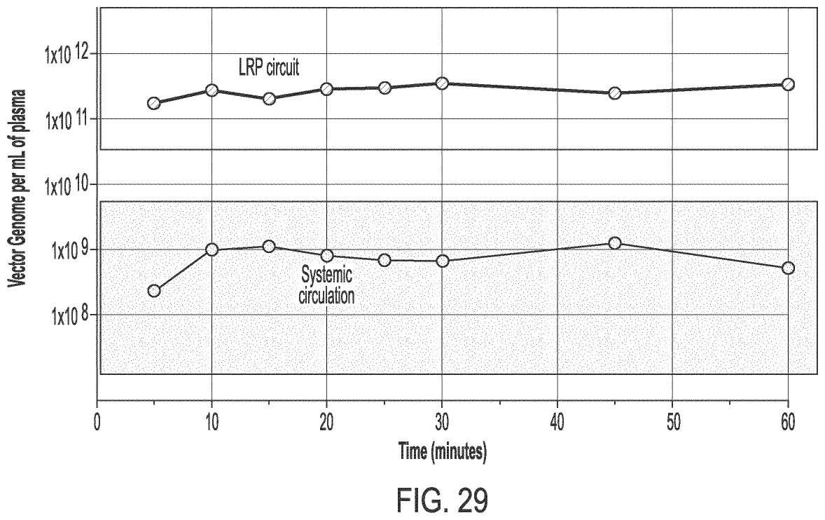

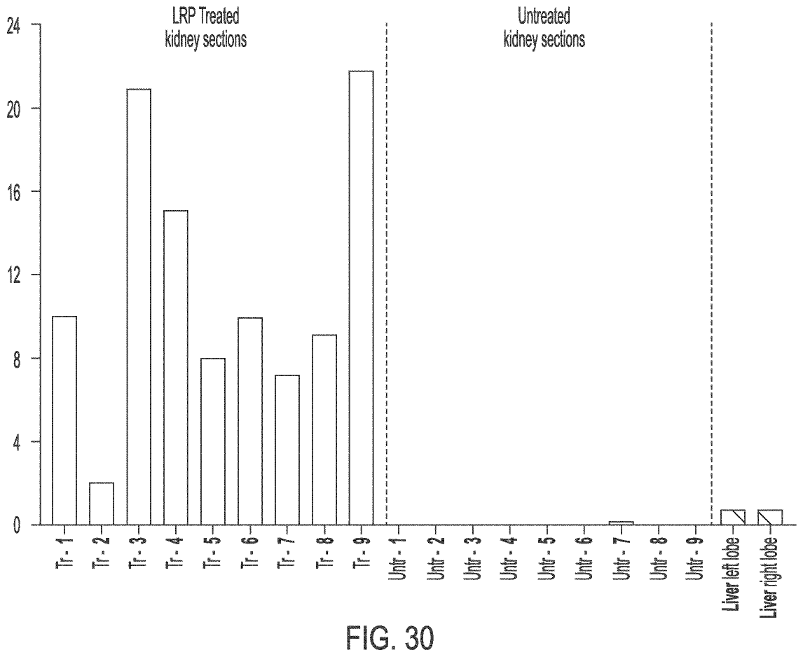

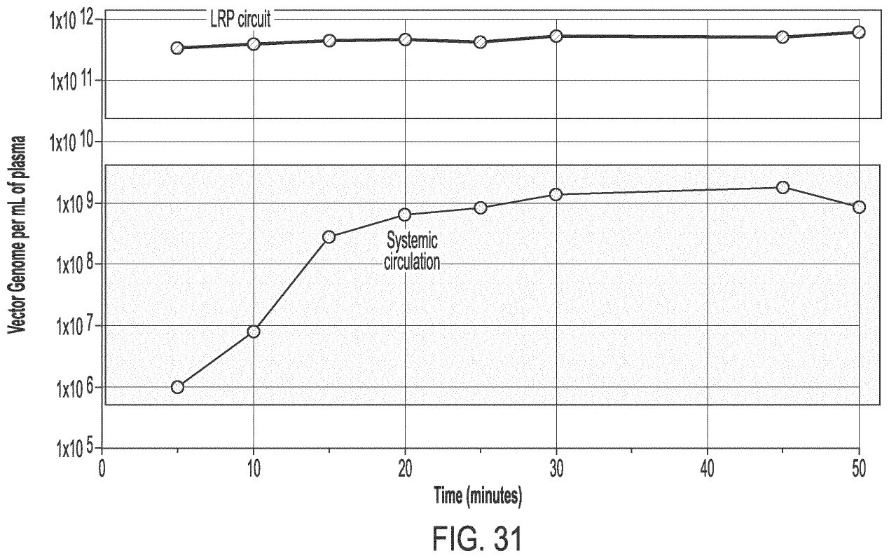

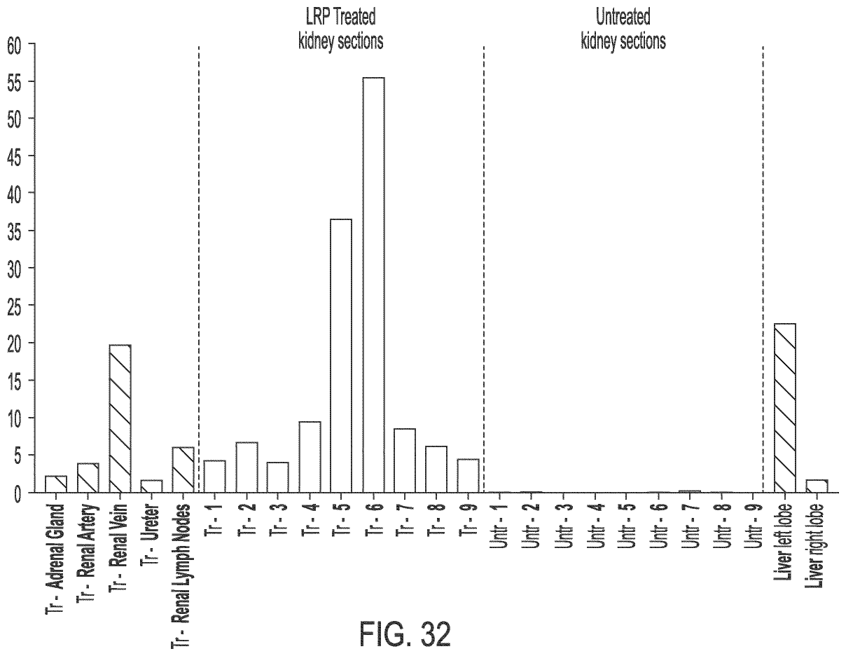

AAV5-CMV-GFP was further evaluated by itself without other serotypes present in porcine kidneys of two different animals: one at a dose of 1.2×10¹³ vg/kg for a 60-minute LRP procedure (FIGS. 29 and 30); and one at a dose of 1.3×10¹³ vg/kg for a 52-minute LRP procedure (FIGS. 31 and 32). Both tests further showed relatively constant concentrations of vector in the LRP circuit and relatively low amounts of leakage of the vector into the systemic circulation, as well as localized biodistribution in the treated kidney with insignificant quantified vector genome in the untreated kidney and liver.

### Example 5: Single-Kidney LRP Procedure for Biodistribution Studies

### 5.1. Materials and Components

An LRP system and protocol for single kidney LRP is now described, which was used to perform the biodistribution studies described below in Examples 6-8.

LRP was performed on farm pigs utilizing the LRP system 1800 illustrated in and described with respect to FIG. 18. Accessory devices/components that were used in the procedure described below are listed in Table 2, including their intended uses and the use in the LRP system in accordance with the embodiments of the disclosure.

TABLE 2
Components used for single kidney LRP procedure

| Item | Brand |
|---|---|
| D-100 OxySet (oxygenator) | Dideco-Livanova |
| Bio-Medicus 540 Bio-Console | Medtronic |
| Blood pump head | Medtronic |
| Pressure Wire X | Abbott |
| Catheter multipurpose 5-Fr: AL 2.0 guide | Cordis |
| catheter | |
| Guidewire V18 | Boston Scientific |
| Hi-Torque Guidewire | Abbott |
| Standard J-tip GW (0.035 cm, 260 mm length) | Cordis |
| (alternative guidewire if required) | |
| Rosen Heavy duty (0.035 cm, 180 mm length) | Cordis |
| (alternative guidewire if required) | |
| Dryseal 22-Fr 33 cm | Gore |
| Dryseal 14-Fr 33 cm | Gore |
| Quantient FFR console | Saint Jude Medical |
| Lunderquist Extra Stiff Wire Guide 0.035, | Cook |
| 260 cm | |
| Tubing ¼ × 1/16 | Medtronic |
| Xvac Vaccum Pump | Sorin |
| Clamp-On Flow Probes | Sonotec |

The supply line catheter assembly comprises a perfusion catheter, a supply dilator, and accessories. The perfusion catheter comprises a reinforced inner shaft and a non-reinforced outer shaft. The inner shaft with an inner diameter of 2.7 mm (though diameters from 2-3.5 mm are contemplated) is sufficiently large to establish a physiological perfusion flow. The catheter is compatible with a 14Fr introducer. The distal section of the shafts is more flexible to enable smooth, atraumatic tracking of the catheter into the renal artery. The flexibility is controlled by the hardness of the Pebax polymer: 72D proximal, 55D transition zone and 35D for the distal section.

The tip of the perfusion catheter is short (2-3 mm) to allow for the balloon to be placed close to a bifurcation in case of a short main stem of the renal artery. The tip is soft (Pebax 35D) and rounded to ensure atraumatic advancement through the vasculature. Further, the inner diameter is slightly reduced to minimize the gap between dilator and tip.

Marker bands are embedded below the balloon and close to the tip to visualize these landmarks under fluoroscopy.

A compliant occlusion balloon is mounted on the outer shaft and is made of polyblend or polyurethane material. The soft balloon gently adapts to the vessel shape for tight sealing. The balloon dimensions are 3-5 mm in length with a disk-like inflation shape with a maximum outer diameter of about 11 mm. The balloon diameter is dependent on the inflation volume, having a linear relationship from about 4 mm inflation diameter at about 0 mL inflation volume to about 11 mm diameter at about 0.5 mL inflation volume. The gap between the inner and outer shaft is used to inflate the balloon, with the outer shaft having multiple inflation holes below the balloon.

A hub is connected to the proximal shaft ends to act as a user interface and to allow for the following accessories to be attached: a luer connector for a syringe to inflate the balloon; a barbed connector to allow for the perfusion tubing to be attached; and a hemostatic valve with luer connector for flushing, where the valve can be used to exchange the dilator with guide and pressure wire with minimal blood loss.

The supply line dilator is placed in the inner lumen of the perfusion catheter before tracking the device. The dilator fills the large inner lumen of the perfusion catheter to allow for atraumatic advancement of the catheter through the vasculature. The outer diameter of the dilator is slightly smaller than the perfusion catheter inner shaft to enable movement of the dilator within the catheter with minimal friction.

The dilator tip is tapered to gently dilate the vessels for the passage of the catheter, and is rounded and atraumatic. The tip inner diameter is about 0.5 mm to minimize the gap to the guide wire. The dilator is compatible with 0.018″ guidewires and smaller. In the proximal section of the dilator its inner diameter is increasing to optimize flexibility. The dilator is made of Pebax 35D and barium sulfate (BaSO₄). Barium sulfate is added to make the catheter radiopaque. In addition, a radiopaque marker may be embedded in the shaft to indicate the tapered section. A colored marking may be added to the proximal dilator section to indicate how the dilator should be placed in the perfusion catheter. A hub is attached to the proximal end of the dilator shaft to ease guidewire insertion. Further, a lure connector allows for syringe attachment for flushing.

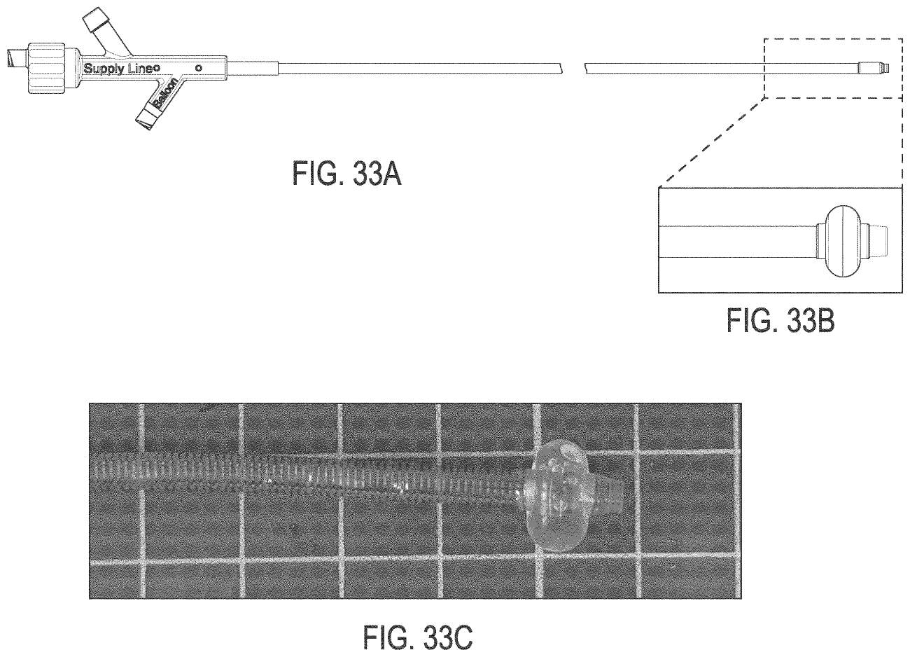

A schematic of the perfusion catheter is shown in FIG. 33A where the balloon is in the retracted state, and FIG. 33B illustrates the balloon in the deployed state. FIG. 33C is a photograph of the perfusion catheter with the balloon in the deployed state.

Other accessories included with the perfusion catheter are now described.
(1) An extension line with 4-way stopcock is delivered with the catheter to be attached to the balloon inflation port at the hub.
(2) A ¼″ tubing assembly is attached to the barbed connector of the hub. The tubing is about 10 cm long. A red on/off tubing clamp is placed over the tubing, which is used to clamp the perfusion flow. The red color indicates to the user that this line carries arterial blood. A T-piece is attached to the other end of the tubing. The T-piece is a straight connector with two ¼″ barbs on the short sides and a luer connector on the long side. The perfusion flow is guided straight from one ¼″ barb to the other. A 4-way stopcock is attached to the luer on the side. This side port is used to de-air, inject contrast media, inject a pharmaceutical composition, and to take samples.
(3) A thin-walled plastic sleeve may be positioned over the distal end and the balloon after production to protect the balloon and its bonds during shipping and shelf-life. The protective sleeve is removed before use of the catheter.

The return line catheter assembly comprises a recovery catheter, a return dilator, and accessories. The return catheter comprises a reinforced inner shaft and outer shaft. The inner shaft with an inner diameter of 4.06 mm (though diameters from 3-4.5 mm are contemplated) is sufficiently large to establish a physiological drainage flow. The catheter is compatible with a 22Fr introducer. The distal shaft section of the shafts is more flexible to enable smooth, atraumatic tracking of the catheter into the renal vein. The shaft flexibility is controlled by the hardness of the Pebax polymer: 72D proximal, 55D transition zone and 35D for the distal section.

The tip of the catheter has lateral holes/perforations (4-8 holes, diameter 0.5-2 mm) to drain through. The tip may be placed close to the vessel wall and the side holes help to avoid the return flow being compromised if the distal opening is fully or partially occluded. The tip length is short (4-8 mm length) to allow for the balloon to be placed close to a bifurcation in case of a short main stem of the renal vein. The tip is made of soft and radiopaque polymer blend (Pebax 35D+BaSO₄) without reinforcement. The distal edge of the tip is rounded to ensure atraumatic advancement through the vasculature. Further, the tip inner diameter is slightly reduced to minimize the gap between dilator and tip.

Marker bands are embedded below the balloon to visualize these landmarks under fluoroscopy.

The compliant occlusion balloon is mounted on the outer shaft and is made of polyblend or polyurethane material. The soft balloon gently adapts to the vessel shape for tight sealing. The balloon dimensions are 8-15 mm in length with a disk-like inflation shape with a maximum outer diameter of about 25 mm. The balloon diameter is dependent on the inflation volume, having a linear relationship from about 6 mm inflation diameter at about 0 mL inflation volume to about 25 mm diameter at about 5 mL inflation volume. The gap between the inner and outer shaft is used to inflate the balloon, with the outer shaft having multiple inflation holes below the balloon.

A hub is connected to the proximal shaft ends to act as a user interface and to allow for the following accessories to be attached: a luer connector for a syringe to inflate the balloon; a barbed connector to allow for the drainage tubing to be attached; and a hemostatic valve with luer connector for flushing, where the valve is used to exchange the dilator with guide and pressure wire with minimal blood loss.

The return line dilator is placed in the inner lumen of the recovery catheter before tracking the device. The dilator fills the large inner lumen of the recovery catheter to allow for atraumatic advancement of the catheter through the vasculature. The outer diameter of the dilator is slightly smaller than the recovery catheter inner shaft to enable movement of the dilator within the catheter with minimal friction.

The dilator tip is tapered to gently dilate the vessels for the passage of the catheter, and is rounded and atraumatic. The tip inner diameter is about 0.95 mm to minimize the gap to the guide wire. The dilator is compatible with 0.035″ guidewires and smaller. In the proximal section of the dilator its inner diameter is increasing to optimize flexibility. The dilator is made of Pebax 35D and BaSO₄, which is added to make the catheter radiopaque. In addition, a radiopaque marker may be embedded in the shaft to indicate the tapered section. A colored marking may be added to the proximal dilator section to indicate how the dilator should be placed in the recovery catheter. A hub is attached to the proximal end of the dilator shaft to ease guidewire insertion. Further, a luer connector allows for syringe attachment for flushing.

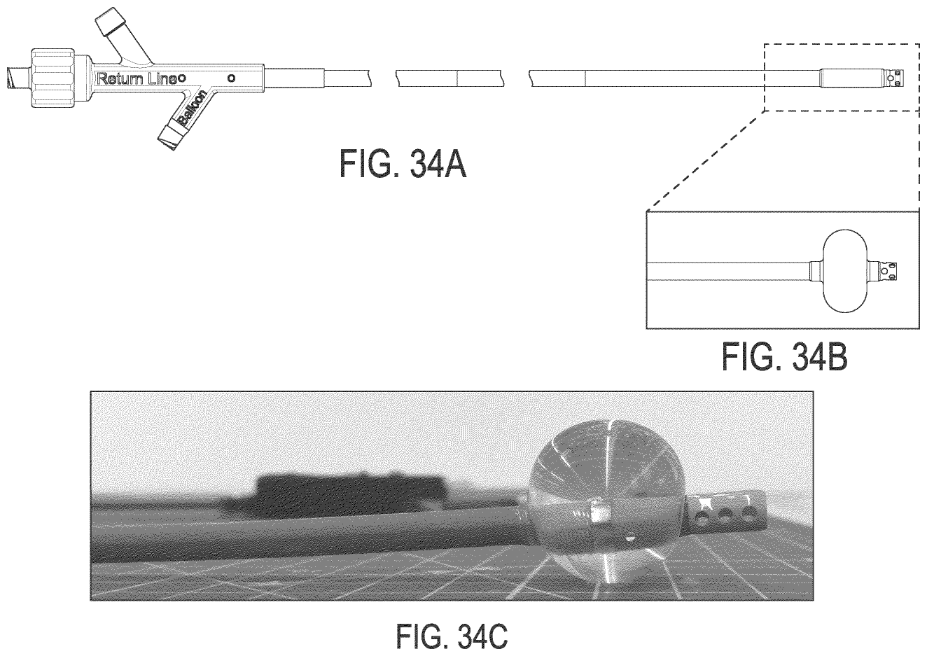

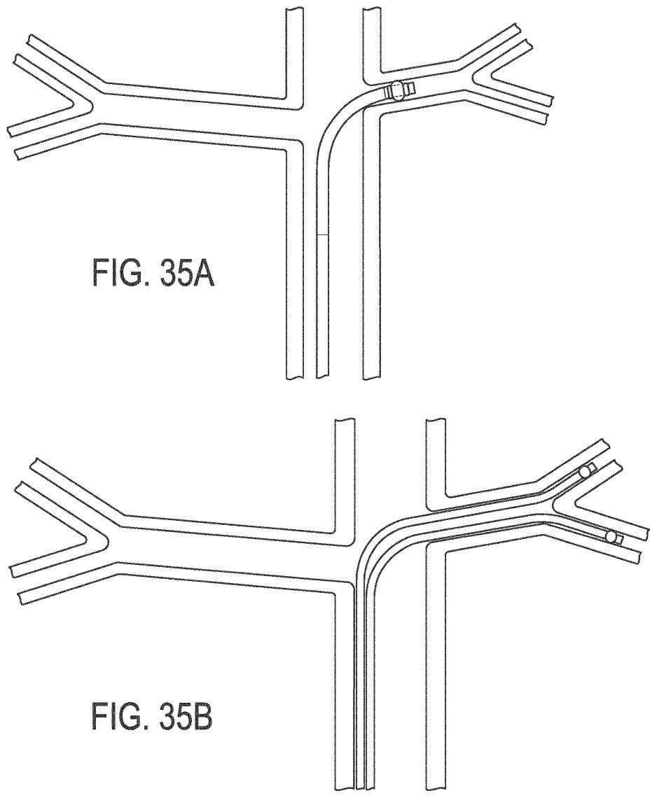

A schematic of the recovery catheter is shown in FIG. 34A where the balloon is in the retracted state, and FIG. 34B illustrates the balloon in the deployed state. FIG. 34C is a photo of the recovery catheter with the balloon in the deployed state.

Other accessories included with the recovery catheter are now described.
(1) An extension line with 4-way stopcock is delivered with the catheter to be attached to the balloon inflation port at the hub.
(2) A ¼″ tubing assembly is attached to the barbed connector of the hub. The tubing is about 10 cm long. A blue on/off tubing clamp is placed over the tubing, which is used to clamp the drainage flow. The blue color indicates to the user that this line carries venous blood. A T-piece is attached to the other end of the tubing. The T-piece is a straight connector with two ¼″ barbs on the short sides and a luer connector on the long side. The return flow is guided straight from one ¼″ barb to the other. A 4-way stopcock is attached to the luer on the side. This side port is used to de-air, inject contrast media, inject a pharmaceutical composition, and to take samples.
(3) A thin-walled plastic sleeve may be positioned over the catheter distal end and the balloon after production to protect the balloon and its bonds during shipping and shelf-life. The protective sleeve is removed before use of the catheter.

The perfusion and recovery catheters with all accessories are fixated on a carton plate with straps, clamps, and protective tubes. The carton plate with the attached devices is placed in a Tyvek pouch and heat sealed. The pouch protects the product from contamination but allows for ethylene oxide (EtO) gas to penetrate for the sterilization process. The pouch is then placed in a robust outer carton box for shipping and storage.

### 5.2 Protocol

The entire LRP procedure can be divided in to four phases: (1) an initiation phase, during which the LRP circuit is not yet established; (2) a stabilization phase, during which the LRP circuit is established; (3) a therapeutic delivery phase, during which the LRP circuit is established; and (4) a removal phase, during which the LRP circuit is discontinued. Each phase is now described.

Initiation phase: interventional placement of arterial and venous catheters in their appropriate position (i.e., into the main left kidney artery and vein, balloon inflation, and assessment of quality of the seal).

Stabilization phase: the start of LRP perfusion without any payload. The stabilization phase starts when the renal artery is anterogradely perfused with oxygenated blood, while the returning de-oxygenated blood is collected from the renal vein via the recovery catheter. The LRP perfusion is considered stable when operators confirm the following metrics: stable reservoir volume, sufficient flow through catheters, physiological renal artery pressures in the absence of pulsatility, stable vacuum pressure, stable rate of glyceryl trinitrate infusion or other vasoactive substances, and potentially complete volume exchange of saline for blood in the reservoir. At this point, payload delivery through the LRP system can commence.

Therapeutic delivery phase: once stabilization phase is concluded, the therapeutic phase begins, and the therapeutic payload is introduced into the LRP system. The duration of this phase is dependent on therapeutic delivery protocol. The total time of therapeutic payload re-circulation is referred to as the “LRP duration.”

Removal phase: once the therapeutic delivery phase is finished according to the pre-defined protocol, LRP perfusion system is stopped, and arterial (perfusion) and venous (recovery) catheters are removed from the body following a specific procedure.

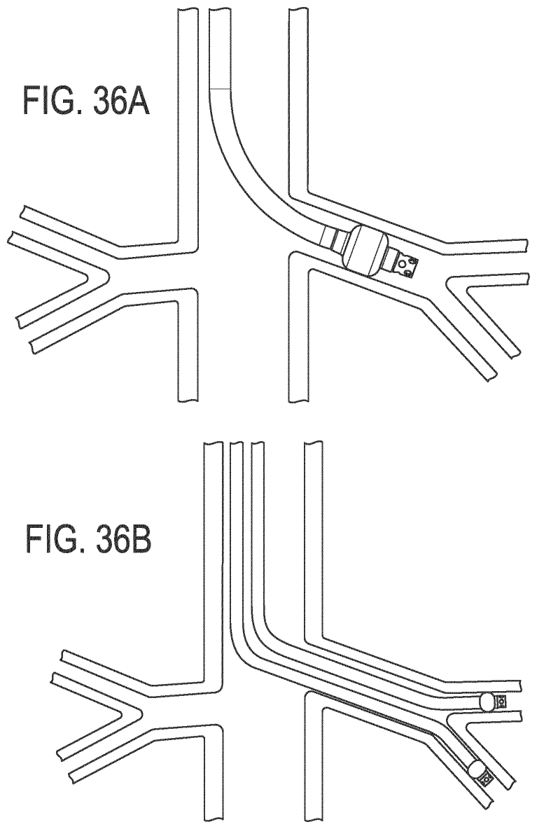

It is noted that, although the protocol discussed below refers to a single perfusion catheter and a single recovery catheter, multiple catheters may be used to optimize the seal of the LRP circuit based on the anatomy of the patient. For example, FIGS. 35A and 35B illustrate the deployment of a single perfusion catheter and a pair of perfusion catheters, respectively, within the renal artery of the left kidney. Similarly, FIGS. 36A and 36B illustrate the deployment of a single recovery catheter and a pair of recovery catheters, respectively, within the renal vein of the left kidney.

A detailed protocol of the LRP procedure that was followed in this example and the following examples is now described:

### Initiation Phase

(1) Place the study animal in dorsal recumbency;
(2) Prepare the jugular central venous catheter in a sterile fashion;
(3) Place surgical drapes and extra drapes in order to cover fully the animal (jugular area and femoral area exposed) as part of sterile access site preparation;
(4) Place an extra set of drapes in order to use them upon “contaminated” catheter removal at the end of the procedure;

### Femoral Artery:

(5) Identify the target vessel under ultrasound guidance in the inguinal region;
(6) Introduce the appropriate femoral introducer sheath (diameter 14-FR);
(7) Flush the catheters with heparinized saline solution;

### Jugular Vein:

(8) Repeat the previous steps (steps 5 to 7) but for the left jugular vein with a 22-FR introducer sheath;

### Catheter Placement:

(9) Place the guide wire (0.014″ or 0.018″, e.g., Boston Scientific V18 or Abbott High-Torque, at the interventionalist's discretion) in the distal renal artery through a multipurpose catheter (5-FR);
(10) Place at least one perfusion catheter in the main stem of the renal artery to ensure homogenous blood distribution to all parts of the kidney and to ensure stable catheter position;
(11) Pass the PressureWire X (Abbott) through the supply line and place it in one of the kidney artery branches;
(12) Visualize the renal arterial anatomy with an antegrade contrast media injection through the perfusion catheter to confirm that the catheter is in the desired position and that only renal structures are perfused by this artery;
(13) Place the Lunderquist guide-wire (or comparable, 0.035″) in the distal renal vein through a 5-FR multipurpose catheter;
(14) Place at least one recovery catheter over the Lunderquist guidewire into its position in the main stem of the left renal vein to ensure homogenous blood drainage from all parts of the kidney and ensure a stable catheter position;
(15) Place a second PressureWire X (Abbott) through the return line for the venous pressure measurement in the renal vein;
(16) Once the proper positioning of the venous catheter is confirmed, inflate the balloon of the recovery catheter;
(17) Assess the renal venous anatomy by retrograde contrast media injection via a diagnostics catheter placed in the lumen of the recovery catheter;
(18) Under fluoroscopy, test the tightness of the balloon occlusion by retrograde injection of the contrast medium. Acceptance criteria for seal quality are: (i) no para-balloon flow or contrast trace; (ii) no filling of other venous structures that are connected to the vena cava; and (iii) no drainage of major extra-renal vessels;
(19) Deflate recovery catheter balloon to allow natural drainage and outlet of injected contrast media;
(20) Prepare the LRP system by the perfusionist team together with the operators;
(21) Prepare the LRP system for LRP procedure:
   a. Prime with 250 mL of heparinized saline
   b. De-air
   c. Turn on the LRP pump and run the circuit in shunt;
   d. Connect the recovery catheter with the reservoir using tubing;
   e. Connect the perfusion catheter via tubing with the arterial outflow of the oxygenator, and completely de-air the tubing and catheter;
   f. Unclamp the venous line and close the shunt;
   g. Begin filling the reservoir by adding blood;
   h. If the system is stable, unclamp the arterial line and fully exchange saline in reservoir with blood (full exchange of volume may be confirmed with hematocrit analysis);
   i. Suction on the venous side and blood pump speed are variable and adapted to the physiological needs on a case by case basis (usually from −80 mmHg to 0 mmHg for vacuum and 2500-4500 rpm for blood pump speed);

### Stabilization Phase

(22) LRP circulation starts with the recover catheter balloon deployed and perfusion catheter balloon retracted;
   a. Deploy the recovery catheter balloon (if desired, re-confirm final position with retrograde contrast media injection);
   b. Check: the tightness and position of the catheters with a contrast injection; the pressure in the kidney, both in the renal artery and renal vein (an acceptable range is 60 mm Hg to 140 mm Hg in the artery, and at or below central venous pressure in the renal vein); the mean arterial kidney pressure versus mean systemic pressure ratio (the ratio should be slightly below 1 to safeguard against leakage); the stability of the reservoir volume (slight volume gain is acceptable, provided that the rate of gain would not result in reservoir overfilling in the allocated LRP time, or hemodynamic instability due to excess blood volume removal from the animal); the RPM of the LRP pump; and the flow through the catheters (a flow between 150 mL/min to 800 mL/min is preferred);
(23) When the circuit is stable and full volume exchange is confirmed by hematocrit analysis, seal the kidney artery with the perfusion catheter under fluoroscopy;
(24) Once both renal vessels are sealed, full LRP circulation is established;
(25) After 5 minutes:
   a. Begin the glyceryl trinitrate (GTN) infusion through the arterial line at a rate of 0 μg/kg body weight/min to 4 μg/kg body weight/min if needed (i.e., in the case of high arterial pressure or renal artery spasms;
   b. Check: the tightness and position of the catheters with a contrast injection; the pressure in the kidney, both in the renal artery and renal vein (an acceptable range is 60 mmHg to 140 mmHg in the artery, and the acceptable range is at or below central venous pressure in the renal vein); the mean arterial kidney pressure versus mean systemic pressure ratio (the ratio should be slightly below 1 to safeguard against leakage); the stability of the reservoir volume (slight volume gain is acceptable, provided that the rate of gain would not result in reservoir overfilling in the allocated LRP time, or hemodynamic instability due to excess blood volume removal from the animal); the RPM of the LRP pump; and the flow through the catheters (a flow between 150 mL/min to 800 mL/min is preferred);
(26) Check all parameters listed above every 5 minutes, until all parameters are in the acceptable range for at least 20 consecutive minutes;

### Therapeutic Delivery Phase

(27) When all major LRP system components are stable for at least 20 minutes, the payload can be delivered. Payload is provided via bolus injection on the venous return line via a 3-way stopcock. Alternative injection methods would be: continuous injection, bolus injection into the supply line, or several boli through either the return or supply lines. After payload is injected, a flush is provided through the same location;
(28) Perform the single kidney LRP procedure for the pre-defined time period;
(29) Every 5 minutes, check: the tightness and position of the catheters with a contrast injection; the pressure in the kidney, both in the renal artery and renal vein (an acceptable range is 60 mmHg to 140 mmHg in the artery, and the acceptable range is at or below central venous pressure in the renal vein); the mean arterial kidney pressure versus mean systemic pressure ratio (the ratio should be slightly below 1 to safeguard against leakage); the stability of the reservoir volume (a slight volume gain is acceptable, provided that the rate of gain would not result in reservoir overfilling in the allocated LRP time, or hemodynamic instability due to excess blood volume removal from the animal); the RPM of the LRP pump; the flow through the catheters (a flow between 150 mL/min to 800 mL/min is preferred);
(30) At T +5 min, +10 min, +15 min, +20 min, +25 min, +30 min, +45 min, and 60 min (or other pre-defined time-points of interest):
   a. blood samples are collected from peripheral blood for blood gas and shedding analyses;
   b. blood samples are collected from LRP system for shedding, vector infectivity analyses, ACT and blood gas analysis;
   c. Urine biochemistry and shedding analysis is performed;

### Removal Phase

(31) At the end of LRP:
   a. Discontinue glyceryl trinitrate, if used;
   b. Prepare a 20 mL syringe, fill it with systemic arterial blood, and attach the syringe to a sideport of the perfusion catheter;
   c. Clamp the arterial line, flush the perfusion catheter lumen with the prefilled syringe through the side port;
   d. Immediately after flushing, deflate the perfusion catheter balloon;
   e. Extract the arterial line out of the body;
   f. During this time, the renal vein balloon has to remain engaged and under suction, filling the ECMO reservoir with the remaining payload containing blood;
   g. After about 150 mL has been drained, clamp the venous line, and retract/deflate the recovery catheter balloon. Extract the venous line out of the body. The catheters are fully disengaged with specific care not to spill payload containing blood;
(32) Discard the entire LRP circuit, reservoir, blood pump and catheters in an appropriate biosafety bin; and
(33) Perform immediate post-surgical care.

### Example 6: Biodistribution of AAV5 Following Administration to the Left Kidney of a Farm Pig Via LRP

### 6.1 Procedure Description and Dosing

A farm pig received kidney LRP (1 h dosing time) treatment, based on the protocol described in Example 5, with AAV5-CMV-eGFP at a dose of 9.8×10¹⁴ vg. This pig was selected out of 12 pigs following immunological screening for AAV5 pre-existing antibodies and computerized tomography (CT) scan of the kidney to ensure that that the pig was physiologically compatible with LRP procedure.

### 6.2 Vector Shedding Analysis

To confirm the tightness of the LRP procedure, multiple blood samples at different time points were collected from the LRP system and peripheral blood (systemic circulation). Urine samples were also collected to estimate virus shedding during the procedure. The total viral genomes per mL in blood and urine samples for this animal was measured using qPCR with probes that target the AAV GFP DNA sequence, and was titrated based on a standard curve using linearized plasmid DNA. All samples were measured in duplicates. Although the pig had a challenging anatomy due to bifurcation of the left renal vein that required additional catheterization, the procedure was successful. There was no leakage from LRP into systemic circulation.

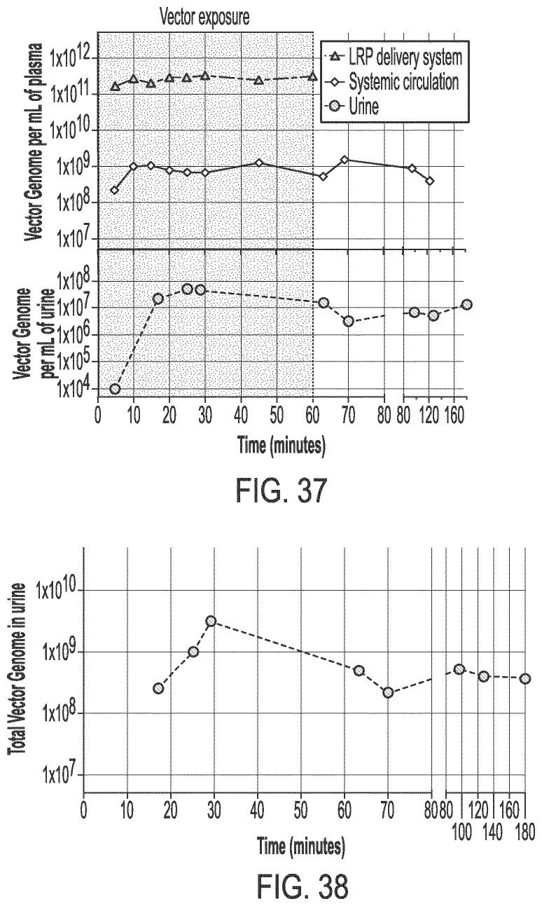

FIG. 37 is a plot showing shedding analysis (vector genome per mL of plasma or urine) detected in the LRP circulation, the systemic circulation, and urine of the treated farm pig (1.2×10¹³ vg/kg, 60 min LRP procedure). FIG. 38 is a plot showing total vector genomes detected in urine of the treated farm pig (1.2×10¹³ vg/kg, 60 min LRP procedure). The viral genome levels remained stable over time until end of procedure, as revealed in FIG. 37. The vector was also found in urine early after LRP-dosing, as revealed in FIG. 38. In this experiment, urine was collected at selected time points, and urine volume was measured before emptying the urine bag. Of note, there were fluctuations in the urine volume during LRP procedure. Therefore, the urine volume was used to calculate the total viral genomes in urine at each time point (FIG. 38). Virus shedding was more prominent after 10 minutes of dosing AAVs into the LRP.

### 6.3 Biodistribution by Region of the Kidney (Cortical, Pyramidal, and Papillary)

A small piece of each tissue was homogenized in a TissueLyser II (Qiagen) using metal beads and extracted using AllPrep DNA/RNA (Qiagen). DNA concentration and purity were measured using a NanoDrop spectrophotometer. Quantification of viral genomes in cells of tissue samples (vg/dg) was performed using droplet digital PCR (ddPCR). A GFP probe was used to detect viral genomes for the animal against a housekeeping gene using a Beta actin probe.

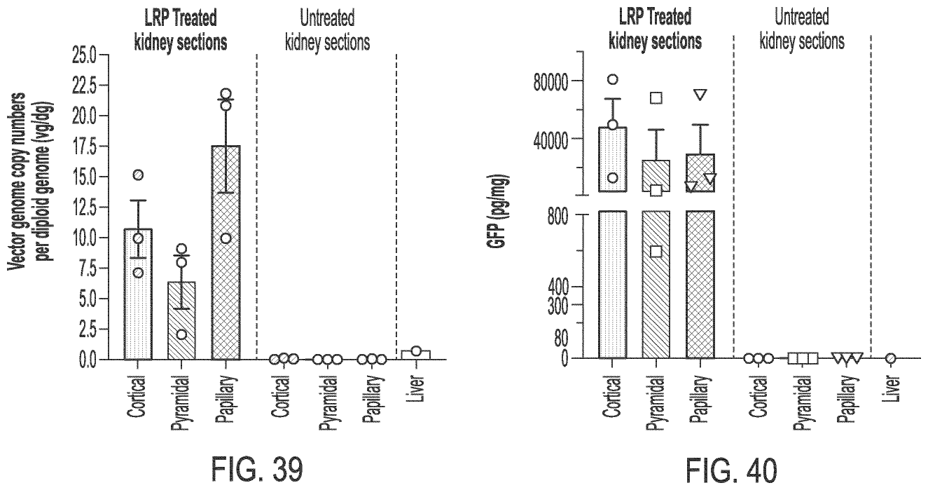

FIG. 39 is a plot showing biodistribution analysis in vg/dg of the LRP-treated kidney (1.2×10¹³ vg/kg, 60 min LRP procedure) divided by kidney sections compared to the untreated kidney and liver. The pig showed a significant increase in AAV5 viral genome in the treated kidney (FIG. 39, mean 11.5) compared to pigs treated with the same dose of AAV9 (highest average observed 0.5 vg/dg). This also confirmed results from a previous study comparing these 2 serotypes co-administered in the same animal at a lower dose (10×; see FIG. 26). The untreated kidney and liver remained highly de-targeted. The observation of comparable levels of transduction throughout the cortical and outer and inner medullary regions (i.e., pyramidal and papillary) indicates that AAV5 delivered via LRP can broadly transduce the cells of the nephron, as well as other cells of the kidney. Given AAV5 delivered by LRP can transduce cells present in regions of both high blood flow (e.g., cortex) and low blood flow (e.g., pyramidal and papillary), and given AAV5 can transit into urine flow within renal tubules and be excreted, AAV5 may be able to access cells of the kidney via vascular circulation as well as from within the renal tubules. These properties make AAV5 a desirable vector to use for delivery of genetic medicine to the kidney using LRP.

6.4 GFP pg/mg of Tissue by ELISA

A GFP SimpleStep ELISA kit (Abcam #ab 171581) was used to quantify the amount of reporter protein in sections of the LRP-treated kidney (AAV5-CMV-GFP). The assay was performed according to manufacturer's instructions. In brief, a spoon (30-50 mg) of tissue powder was homogenized in chilled extraction buffer. After centrifugation, the supernatant was collected into clean tubes und further processed. Standards were freshly prepared, and samples were run in duplicate. After incubation with antibody cocktail and extensive washing, the substrate was added, and the reaction was stopped before saturation. Optical density (OD) measurements were recorded at 450 nm and converted to pg/mg, after normalization to total protein concentration.

FIG. 40 is a plot showing GFP protein levels in pg/mg in the LRP-treated kidney (1.2×10¹³ vg/kg, 60 min LRP procedure) compared to untreated kidney and liver. Consistent with the observation of high levels of transduction throughout the treated kidney, high levels of GFP expression were detected throughout the cortical and outer and inner medullary regions of the treated kidney. No GFP expression was detected in the untreated kidney or liver, further demonstrating effective transduction of only the LRP-treated kidney. The observation of comparable levels of GFP expression throughout the cortical and outer and inner medullary regions (i.e., pyramidal and papillary) indicates that AAV5 delivered via LRP can broadly transduce and provide transgene expression in the cells of the nephron and as well as other cells of the kidney. These properties further make AAV5 a desirable vector to use for delivery of genetic medicine to the kidney using LRP.

### 6.5 Description of Transduced Cells Observed Via RNAScope

The in situ hybridization technology assay (ACD, a BioTechne brand) allows for spatial visualization of single mRNA or episomal DNA molecules. To improve the signal-to-noise ratio, RNAscope employs target specific probes combined to multiple signal amplifiers. This results in punctuated dots which can be visualized with an optical microscope. Here, a specific GFP probe was used in combination with other probes specific to different cell types in the kidney (CDH2-proximal tubules; CDH1-distal tubules; NPHS1-podocytes; SLC12A1-macula densa; PECAM1-endothelial cell marker). This allowed for identification of transduced renal cells after the LRP procedure. In brief, cryosections from fresh-frozen kidney segments were fixed in 4% paraformaldehyde and underwent a cascade of hybridization events with several washes in between. After mounting, slides were imaged with an inverted Axio Observer microscope (Zeiss). Overview pictures of cortical sections showed a homogenous staining of glomeruli in the treated kidney. No signal was observed in the untreated kidney or liver. Additional spots were observed in other tubular structures within the cortex and medullary regions, supporting the ability of AAV5 to transduce a range of cells of the kidney.

### Example 7: Comparison of Biodistribution of AAV5 Following LRP Delivery to the Left Kidney Versus IV Administration

### 7.1 Description of Procedure and Dosing

The aim of this example was to compare two routes of administration of AAV5 to the kidney, kidney LRP dosed for 1 h and intravenous injection (IV), to evaluate the benefits of AAV administration via kidney LRP over IV.

As with Example 6, the two farm pigs used in this study were selected based on immunological screening for AAV5 pre-existing antibodies and CT scans of the kidneys to ensure that the pigs were physiologically compatible with the LRP procedure.

Each animal was dosed with a total of 9.9×10¹⁴ vg of AAV5-CAG-eGFP. Administration of AAV5-CAG-eGFP to Animal 1 via LRP was as described in Example 5. For Animal 2, AAV5-CAG-eGFP was administered as a bolus dose via a central venous access line. Methodology was kept consistent for Animal 1 and Animal 2, except that, for Animal 2, only peripheral blood and urine samples were collected for shedding analysis as no LRP circuit was established in this animal.

### 7.2 Vector Shedding Analysis

To confirm the tightness of the LRP procedure, multiple blood samples at different time points were collected from the LRP system of Animal 1 and peripheral blood (systemic circulation) from both animals. Urine samples were also collected to estimate virus shedding during the LRP procedure. The total viral genomes per mL in blood and urine samples were measured using qPCR with probes that target the AAV GFP DNA sequence and were titrated based on a standard curve using linearized plasmid DNA. All samples were measured in duplicates.

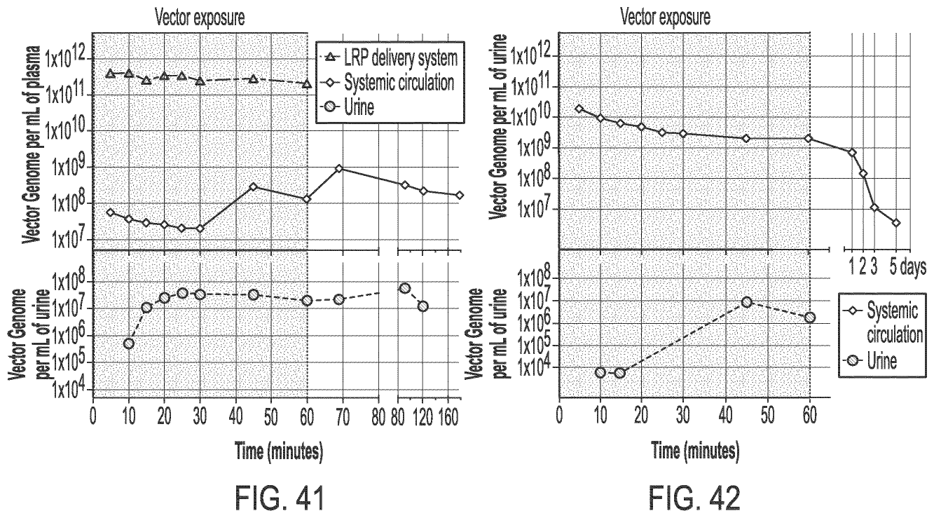

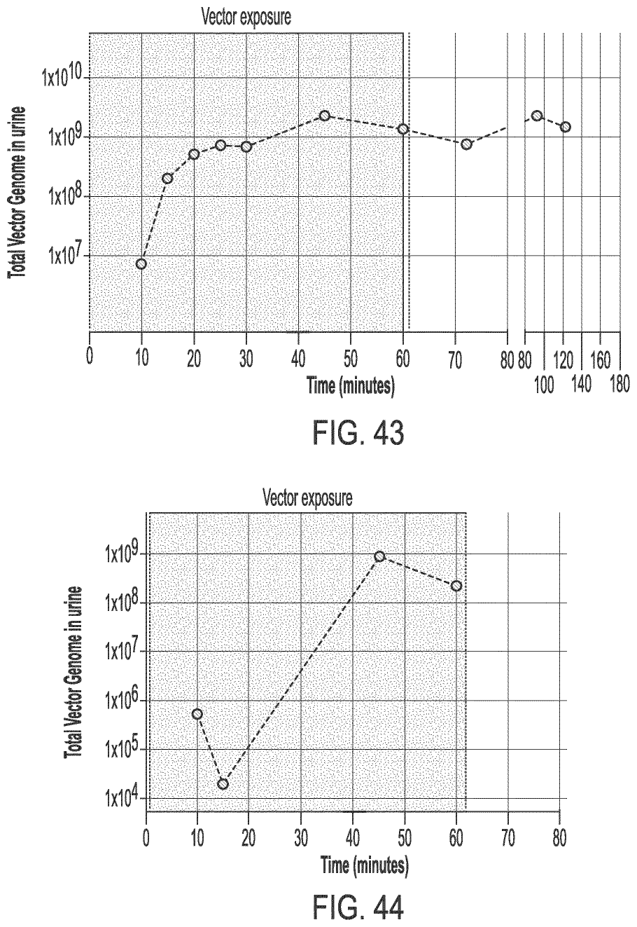

FIG. 41 is a plot showing shedding analysis (vector genome per mL of plasma or urine) detected in the LRP circuit, systemic circulation, and urine of Animal 1. The LRP procedure was successful in Animal 1, with signs of nominal leakage from LRP into systemic circulation in the latter portion of the procedure. The viral genome levels within the circuit remained relatively stable over time until the end of the procedure. The vector was also found in urine early after LRP-dosing, as shown in FIGS. 41 and 43. In this experiment, urine was collected at selected time points, and urine volume was measured before emptying the urine bag. Of note, there were fluctuations in the urine volume during the LRP procedure. Therefore, urine volume was used to calculate the total viral genomes in urine at each time point. Virus shedding into the urine was prominent beginning within 10 minutes of dosing AAVs into the LRP circuit and remained stable throughout the procedure.

FIG. 42 is a plot showing shedding analysis (vector genome per mL of plasma or urine) detected in the systemic circulation and urine of Animal 2. As shown, IV administration of AAV5 to Animal 2 provided a very different exposure profile. In the systemic circulation, the concentration of AAV5 reached during the first hour post-administration was approximately 100-fold higher than observed in Animal 1 during the LRP procedure, indicating much greater systemic exposure following IV versus LRP administration. Viral genomes in Animal 2 persisted in circulation for all time points measured out to day 5 post procedure. The concentration of viral genomes dropped rapidly in the systemic circulation, decreasing by 1 log from the earliest time point post-injection at 5 min to 60 min, 1.94×10¹⁰ vg/mL to 1.93×10⁹ vg/mL, respectively, and continued to decline through day 5 to 3.43×10⁶ vg/mL. Assuming the AAV5 exposure to the kidney is represented by the systemic AAV concentration in Animal 2, the maximum concentration of AAV5 exposure to the kidney was approximately 20-fold less than following administration of the same dose to Animal 1 by LRP (1.94×10¹⁰ vg/mL versus 4.23×10¹¹ vg/mL, respectively). Given the potential of AAV5 to transduce cells of the nephron via exposure from circulation, the substantial difference in the maximum concentration of AAV5 viral genomes exposed to the kidney following LRP versus IV administration could have important implications for delivery of gene therapies to the kidney via AAV5-mediated delivery.

Likewise, the concentration profile of AAV5 viral genomes in urine was substantially different following IV administration to Animal 2 as compared to direct administration to the kidney via LRP in Animal 1. In Animal 2, viral genomes were only evident (8.84×10⁸ vg) at 45 mins post-injection, as shown in FIGS. 42 and 44. Given the potential of AAV5 to transduce tubular epithelial cells of the nephron via the apical (urine facing side) side of these cells, the substantial difference in viral genomes in the urine following LRP versus IV administration could have further important implications for delivery of gene therapies to the kidney via AAV5-mediated delivery.

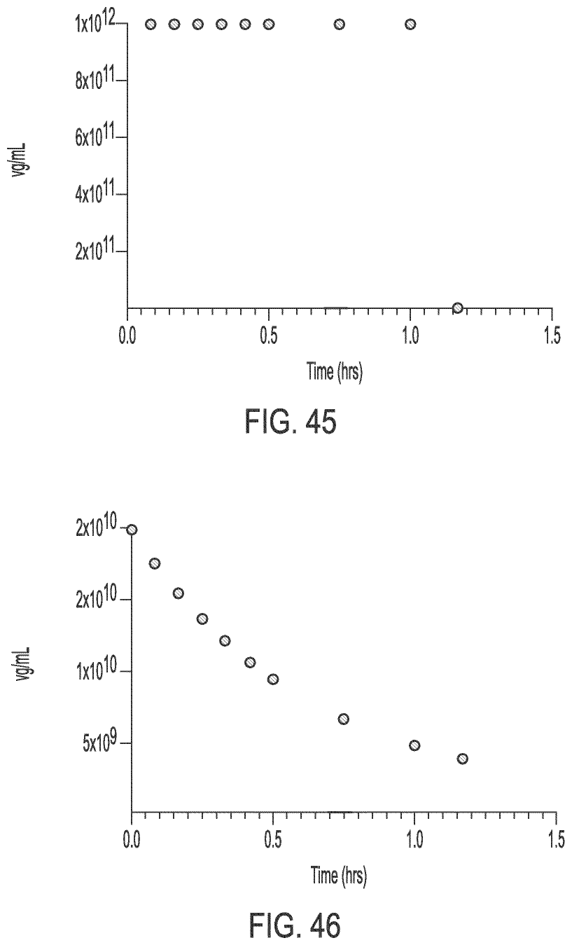

The LRP shedding data obtained from Animal 1 and systemic concentration data from Animal 2 were used to further investigate the differences in AAV5 exposure to the kidney following LRP versus IV administration. FIG. 45 is a plot modeling concentration vs. time for AAV5 in the LRP circuit of the treated kidney of Animal 1, derived from data provided in FIG. 41, which is used to model kidney exposure to viral genomes by accounting for area under the curve (AUC). For LRP modeling, plots were derived using the approximate AAV5 Cₘₐₓ observed in the LRP circuit of Animal 1 and assuming no leakage so a constant AAV5 concentration throughout the procedure. For IV modelling, the assumption was made that circulating concentration of AAV5 in the peripheral blood was representative of exposure to the kidneys following IV administration. The systemic AAV5 concentration data through day 5 was fit using a single-phase exponential decay equation (GraphPad Prism) to obtain the decay constant for AAV5 following IV administration to the pig (k=0.4/hr).

FIG. 46 is plot modeling concentration vs. time for AAV5 in systemic circulation of Animal 2 following IV administration, derived from data provided in FIG. 42, which is used to model kidney exposure to viral genomes by accounting for area under the AUC. The decay curve was used to model the concentration versus time profile of AAV5 for a period equivalent to LRP treatment following IV administration, using the approximate systemic Cₘₐₓ observed in Animal 2 and the obtained decay constant using a single-phase exponential decay model.

AUC analysis of the respective model plots (GraphPad Prism) demonstrated an approximate 200-fold greater exposure of AAV5 to the treated kidney during the LRP treatment phase when using LRP administration as compared to IV administration over the same time period. In principle, the greater exposure of AAV5 to the kidney obtained via LRP delivery will lead to substantially greater transduction of the kidney than IV administration. Additionally, the large difference in systemic exposure when using these respective routes of administration is expected to provide minimal transduction of other organs (e.g., the liver or spleen) following direct administration to the kidney via LRP as compared to IV administration. Given the known safety issues related to AAV transduction of the liver, reducing transduction of non-target organs has substantial safety implications.

### 7.3 Biodistribution by Region of the Kidney (Cortical, Pyramidal, and Papillary)

Small piece of each tissue was homogenized in a TissueLyser II (Qiagen) using metal beads and extracted using AllPrep DNA/RNA (Qiagen). DNA concentration and purity were measured using a NanoDrop spectrophotometer. Quantification of viral genomes in cells of tissues (vg/dg) was performed using ddPCR. A GFP probe was used to detect viral genomes for the animals against a housekeeping gene using Beta actin probe.

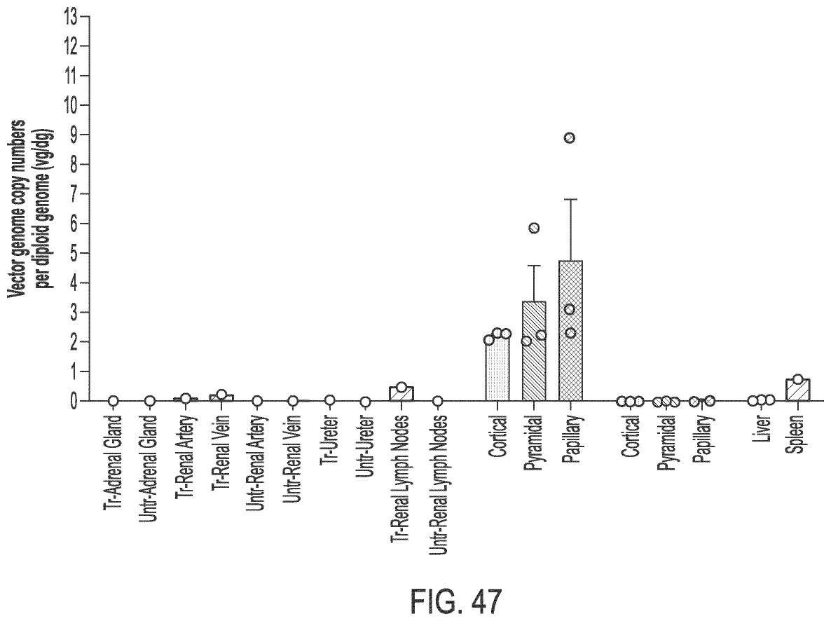

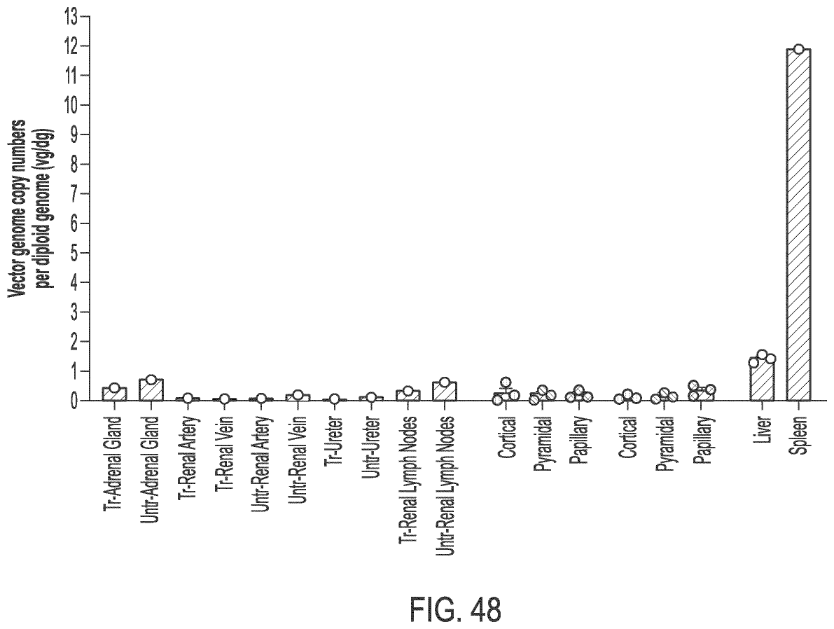

FIG. 47 is a plot showing biodistribution analysis (vg/dg) measured in kidney sections compared to other organs from Animal 1, and FIG. 48 is a plot showing biodistribution analysis (vg/dg) measured in kidney sections compared to other organs from Animal 2 (where Tr=treated, and Untr=untreated). For Animal 1 in which AAV5 was administered to the left kidney via LRP, the biodistribution analysis confirmed the tightness of the LRP system. The viral genomes were primarily detected in the treated kidney sections (left Kidney) with 3.5 vg/dg on average (FIG. 47). The untreated kidney, liver and spleen remained highly de-targeted (untreated kidney 0.01 vg/dg on average; average of three liver samples 0.05 vg/dg; spleen 0.76 vg/dg). By comparison, the biodistribution of AAV5 following IV administration to Animal 2 was substantially different. As anticipated by the greatly reduced exposure of AAV5 to the kidney, transduction of kidney following IV administration was extremely inefficient, with both the left and right kidney showing similar, low average viral genomes of 0.2 vg/dg (FIG. 48). Further, as anticipated from the substantially greater systemic exposure following IV administration of AAV5, viral genomes in Animal 2 were significantly higher in the liver and spleen (1.4 vg/dg and 12 vg/dg, respectively).

To quantify the specificity of target kidney vs non-target organ transduction obtained by LRP administration of AAV5 as compared to IV administration, the ratios of kidney to liver transduction in Animal 1 and Animal 2 were computed and summarized in Table 3. Specificity of targeting was computed by comparing the ratio of kidney to liver of Animal 1 versus Animal 2, calculated based on vg/dg. Specificity of targeting accounting for the difference in cell number per organ was computed using organ size as an estimate of cell number of the corresponding organs.

For Animal 1 the ratio of kidney to liver transduction was 63.7, whereas for Animal 2 this ratio was 0.14. Comparing these ratios demonstrates that LRP administration provides an approximate 450-fold greater specificity of kidney versus non-kidney targeting as compared to IV administration. Notably, given the substantially greater size of the liver than the kidney, this analysis provides an average of transduction specificity at the cellular level without consideration of the substantially greater number of cells within the liver as compared to the kidney. In humans, the volume of a non-diseased liver is approximately 12-times the volume of a non-diseased kidney (Nawaratne et al., “Relationships among liver and kidney volumes, lean body mass and drug clearance,” Br. J. Clin Pharmacol, 1998, Vol. 46, No. 5, 447-452). Using the relative volume of the human liver as compared to the human kidney as an approximation of relative cell number provides an approximate 5400-fold greater specificity of kidney versus non-kidney targeting when AAV5 is administered via LRP as compared to IV administration.

These findings demonstrate the administration of AAV5 via LRP confers an unexpected benefit of substantial transduction of the treated kidney not obtainable by administration of the same dose via IV. Further, AAV5 administration by LRP provides substantial de-targeting of the liver and the spleen, conferring a potential safety benefit in addition to the potential therapeutic benefit obtained by effective transduction of the kidney.

TABLE 3
Summary of main parameters and conclusions of dosing
two farm pigs with AAV5 using LRP versus IV.

| | Animal 1 | Animal 2 |
|---|---|---|
| Method | LRP | IV |
| AAV serotype | AAV5 | AAV5 |
| Total Dose (vg) | ~1 × 10¹⁵ | ~1 × 10¹⁵ |
| Dosing Time (Mins) | 60 | — |
| Incubation (Weeks) | 3 | 3 |
| Kidney (vg/dg) | 3.46 | 0.21 |
| Liver (vg/dg) | 0.05 | 1.42 |
| Spleen (vg/dg) | 0.76 | 11.91 |
| Ratios | | |
| Kidney/Liver | 63.70 | 0.14 |
| Specificity of Targeting | 450 | — |
| Specificity of Targeting | 5400 | — |

### Example 8: Neutralizing AAV Antibody Generation Following Tight Versus Leaky AAV Administration to the Kidney of a Farm Pig Via LRP

### 8.1 Procedure Description and Dosing

The purpose of this analysis was to compare systemic AAV neutralizing antibody formation following administration of AAV via kidney-LRP when a closed circuit was successfully maintained as compared to when substantial leakage was observed during the LRP procedure. Animals (referred to in this example as Animals 3 and 4) were treated with AAV9 for this comparison, as AAV9 systemic exposure is known to trigger a relatively strong immune response. Animal 3 received kidney LRP (1 h dosing time) treatment with AAV9-CMV-eGFP at a dose of 6.2×10¹⁴ vg, while Animal 2 received kidney LRP (2 h dosing time) treatment with AAV9-CMV-eGFP at dose of 1.7×10¹⁵ vg. Both animals were selected following immunological screening for AAV9 pre-existing antibodies and CT scans of the kidneys to ensure that the pigs were physiologically compatible with LRP procedure.

### 8.2 Vector Shedding Analysis

To confirm the tightness of the LRP procedure, multiple blood samples at different time points were collected from the LRP circuit and peripheral blood (systemic circulation). The total viral genomes per mL in blood were measured using qPCR with probes that targeted the AAV GFP DNA sequence, and was titrated based on a standard curve using linearized plasmid DNA. All samples were measured in duplicates.

To measure neutralizing antibodies to AAV9, 2V6.11 cells were seeded in a 96-well white plate and incubated overnight with Ponasterone A. The next day, serum samples from the AAV9 treated pigs were serially diluted and incubated with the AAV9 vector including a reporter gene for 1 hour, and then the mix of serum sample/vector was added to 2V6.11 cells. After 24 hours of incubation, the percentage of AAV9 transduction was evaluated on lysed cells by adding the reporter substrate. Results are reported as the titer required to provide 50% inhibition of transduction. Positive and negative controls were included in the assay, and cells receiving medium only were used to assess the background signal.

The LRP circuit remained tight during the procedure for Animal 1, with no evidence of systemic leakage, and with maximum concentration in the peripheral blood sample (systemic circulation) of 1.7×10⁸ vg/mL. In comparison, significant leakage of AAV9 into the peripheral blood was observed during the LRP procedure for Animal 2, with concentrations rising from 6.6×10⁹ vg/mL at 5 minutes into the procedure to a maximum of 1.2×10¹¹ vg/mL during the procedure.

Anti-AAV9 Neutralizing antibody titers were measured on the day of AAV9 administration to the kidney via LRP (Day 0) and the day of sacrifice (Day 15), with the results being summarized in Table 4. Both animals exhibited a minor amount of anti-AAV9 neutralizing antibody prior to administration of AAV9 via kidney LRP. At Day 15, the anti-AAV9 neutralizing antibody titer for Animal 4 increased significantly to > 1/4096, the highest dilution of serum evaluated. By contrast, the anti-AAV9 neutralizing antibody titer for Animal 3 remained low ( 1/32). Given the safety concerns associated with immune responses to AAV, this analysis demonstrates a potential benefit of tight, closed loop AAV administration to the kidney via LRP. Additionally, the low systemic neutralizing antibody response observed in Animal 3 opens the possibility of redosing with the same AAV should future doses be required.

TABLE 4
Summary of neutralizing AAV9 antibody titers
observed in Animal 3 and Animal 4
Anti-AAV9 Neutralizing Titers

| Animal | Day 0 | Day 15 |
|---|---|---|
| 3 | ¼ | 1/32 |
| 4 | 1/64 | > 1/4096 |

### Example 9: Promoters for Use in Therapeutic Constructs

### 9.1 Exemplary Promoters

Therapeutic constructs in accordance with various embodiments of the present disclosure advantageously incorporate promoter sequences to increase expression activity in renal cells. Such promoters that are contemplated for use in combination with a GOI include, but are not limited to, CMV promoter (508 bp, SEQ ID NO: 9), CAG promoter (1733 bp, SEQ ID NO: 10), NPHS2 promoter (2589 bp, SEQ ID NO: 11), a miniature NPHS2 promoter (265 bp, SEQ ID NO: 12), and KSP-derived promoters such as mKSP0.3 promoter (324 bp, SEQ ID NO: 13) and mKSP1.3 promoter (1341 bp, SEQ ID NO: 14).

9.2 Identifying Miniature Human Kidney-Specific Cadherin (Ksp-Cadherin, Cadherin 16) Promoters while Considering Predictions of DNA Sequences for Transcription Factor Binding Sites

In this example, transcription factor binding sites (TFBS) were predicted based on UCSC Genome Browser, which represents genome-wide predicted binding sites for transcription factor binding profiles in the JASPAR CORE database. This open-source database contains a curated, non-redundant set of binding profiles derived from published collections of experimentally defined TFBS for eukaryotes.

JASPAR 2018 used the TFBS Perl module (Lenhard and Wasserman, Bioinformatics, 2002, Vol. 18) and FIMO (Grant, Bailey, and Noble, Bioinformatics, 2011, Vol 27), as distributed within the MEME suite (version 4.11.2) (Bailey et al., Nucleic Acids Res., 2009, Vol. 37). For scanning genomes with the BioPerlTFBS module, profiles were converted to a position weight matrix (PWM) and matches were kept with a relative score≥0.8. For the FIMO scan, profiles were reformatted to MEME motifs and matches with a p-value<0.05 were kept. TFBS predictions that were not consistent between the two methods (TFBS Perl module and FIMO) were removed. The remaining TFBS predictions were colored according to their FIMO p-value to allow for comparison of prediction confidence between different profiles. For JASPAR 2020, DNA sequences were scanned with JASPAR CORE TF-binding profiles for each taxa independently using PWMScan. TFBS predictions were selected with a PWM relative score≥0.8 and a p-value<0.05. P-values were scaled between 0 (corresponding to a p-value of 1) and 1000 (p-value≤10-10) for coloring of the genome tracks and to allow for comparison of prediction confidence between different profiles. JASPAR 2022 contains updated transcription factor binding sites with additional transcription factor profiles.

Analysis considered the following transcription factors in promoter design, which are known to regulate kidney tubular cells. Examples include:
(1) HNF-1β (Hepatocyte Nuclear Factor 1β): HNF-1β is essential for the development and maintenance of renal tubular cells, particularly in the proximal tubules. Mutations in the HNF-1β gene have been associated with renal cysts and diabetes syndrome (RCAD).
(2) HNF-4α (Hepatocyte Nuclear Factor 4α): HNF-4α is another important transcription factor involved in the regulation of renal tubular cells. It is expressed in the proximal tubules and plays a role in the differentiation and function of these cells.
(3) Pax2 (Paired Box Gene 2): Pax2 is a transcription factor that is crucial for the development of various renal structures, including the tubular system. It is expressed in the early stages of kidney development and helps in the formation and patterning of different tubular segments.
(4) Foxi1 (Forkhead Box I1): Foxi1 is a transcription factor that is important for the development and function of the distal tubules and collecting ducts in the kidney. It regulates the expression of various genes involved in electrolyte balance and acid-base regulation.
(5) HNF-1α (Hepatocyte Nuclear Factor 1α): HNF-1α is expressed in both proximal and distal tubules and plays a role in maintaining tubular integrity and function. Mutations in the HNF-1α gene are associated with a form of maturity-onset diabetes of the young (MODY) that can lead to kidney dysfunction.
(6) KLF5 (Krüppel-like factor 5): KLF5 is a transcription factor that is involved in the regulation of cell proliferation and differentiation in various tissues, including the kidney. It is expressed in renal tubular cells and has been implicated in kidney injury and repair processes.
(7) Pax8 (Paired Box Gene 8): Pax8 is a transcription factor that is expressed in the developing and mature kidney. It plays a role in the specification and differentiation of different tubular segments, including the proximal and distal tubules.
(8) Eya1 (Eyes absent homolog 1): Eya1 is a transcription factor that forms a complex with Pax proteins, including Pax2 and Pax8. It is essential for the development of the metanephric kidney and regulates the expression of genes involved in tubular development and differentiation.
(9) AQP2 (Aquaporin 2): While AQP2 is primarily known as a water channel protein, its expression is regulated by transcription factors. Several transcription factors, including HNF-1β, CREB (CAMP response element-binding protein), and NFAT (nuclear factor of activated T cells), have been implicated in the regulation of AQP2 expression in renal tubular cells.
(10) Sp1 (Specificity protein 1): Sp1 is a transcription factor that regulates the expression of numerous genes involved in various cellular processes. It is expressed in renal tubular cells and has been shown to modulate the expression of genes related to tubular function, such as aquaporins and transporters.
(11) PPARγ (Peroxisome proliferator-activated receptor gamma): PPARγ is a transcription factor that belongs to the nuclear hormone receptor superfamily. It is expressed in renal tubular cells and is involved in the regulation of lipid metabolism and inflammation. PPARγ activation has been shown to protect against kidney injury and fibrosis.
(12) STAT3 (Signal transducer and activator of transcription 3): STAT3 is a transcription factor that is activated in response to various signaling pathways, including cytokines and growth factors. It plays a role in kidney tubular cell survival, regeneration, and inflammation. Activation of STAT3 has been associated with protection against kidney injury.
(13) FOXC1 (Forkhead Box C1): FOXC1 is primarily expressed in the developing kidney during embryogenesis and continues to be expressed in various kidney structures, including tubular cells, throughout development. Its functions in kidney tubular cells include epithelial cell differentiation, epithelial-mesenchymal transition (EMT) regulation, and cilia development and function.
(14) FOXC2 (Forkhead Box C2): FOXC2 is also expressed in the developing kidney and continues to be expressed in various kidney compartments, including tubular cells. Its functions in kidney tubular cells include epithelial cell differentiation, cell migration and adhesion, epithelial polarity and tight junction formation, regulation of Wnt signaling, and lymphatic vessel development.

Based on the foregoing analysis, the following sequences were identified in the human genome (CDH16 candidate promoter region (GRCh38/hg38 reference genome)):
(Seq A) chr16:66920195-66920275 (81 bp, SEQ ID NO: 15) (upstream promoter region with TFBS);
(Seq B) chr16:66920085-66920275 (191 bp, SEQ ID NO: 16) (extended upstream promoter region with TFBS to include conserved region in vertebrates and mammals);
(Seq 1) chr16:66918886-66919090 (SEQ ID NO: 17, 205 bp) (minimal promoter starting at TSS including mouse KSP blasted sequences);
(Seq 2) chr16:66918850-66919090 (SEQ ID NO: 18, 241 bp) (minimal promoter extended downstream to TSS to include conserved region in vertebrates and in particular in mammals);
(Seq 3) chr16: 66918850-66919173 (SEQ ID NO: 19, 324 bp) (minimal promoter extended up- and downstream to TSS to include conserved region in vertebrates and mammals);
(Seq 4) chr16:66918850-66919344 (SEQ ID NO: 20, 495 bp) (minimal promoter extended up- and downstream to TSS to include conserved region in vertebrates and mammals, in particular upstream extension);

Table 5 summarizes candidate mini KSP promoters based on the identified sequences and combinations thereof:

TABLE 5
Candidate mini KSP promoters contemplated
for use in therapeutic constructs

| | Identified | |
|---|---|---|
| Name | Sequence/Combination | Sequence ID |
| mhKSP1 | Seq 1 (205 bp) | SEQ ID NO: 17 |
| mhKSP2 | Seq 2 (241 bp) | SEQ ID NO: 18 |
| midhKSP1 | Seq 3 (324 bp) | SEQ ID NO: 19 |
| midhKSP2 | Seq 4 (495 bp) | SEQ ID NO: 20 |
| mhKSP5 | Seq A + Seq 1 (286 bp) | SEQ ID NO: 21 |
| midhKSP4 | Seq B + Seq 1 (396 bp) | SEQ ID NO: 22 |
| mhKSP3 | Seq A + Seq 2 (322 bp) | SEQ ID NO: 23 |
| mhKSP4 | Seq B + Seq 2 (432 bp) | SEQ ID NO: 24 |
| midhKSP5 | Seq A + Seq 3 (405 bp) | SEQ ID NO: 25 |
| midhKSP6 | Seq B + Seq 3 (515 bp) | SEQ ID NO: 26 |
| midhKSP7 | Seq A + Seq 4 (576 bp) | SEQ ID NO: 27 |
| midhKSP3 | Seq B + Seq 4 (686 bp) | SEQ ID NO: 28 |

### Example 10: Construct Design

Described below are exemplary gene construct designs contemplated for expressing a functional PC1 protein subunit or PC2 protein via localized gene replacement to treat ADPKD. Each construct shown in Table 6 below includes a gene cassette comprising a promoter operatively linked to the GOI (which encodes tdTomato or zsGreen fluorescent protein), an optimized posttranscriptional regulatory element (oPRE), and a terminator sequence (SEQ ID NO: 29), with the gene casettes being flanked by 5′ and 3′ ITR sequences.

Testing of the constructs may be performed using the tdTomato or zsGreen fluorescent proteins as reporters. Once efficient protein expression is verified, the reporter gene can be exchanged with a GOI (e.g., SEQ ID NO. 4, SEQ ID NO: 6, or SEQ ID NO: 8), as would readily be appreciated by those of ordinary skill in the art. For further testing, the gene or interest may further encode an HA tag having the amino acid sequence of SEQ ID NO: 30, for which the corresponding nucleic acid sequence is SEQ ID NO: 31. One or more HA tag sequences can be included, for example, at the N-terminus of the therapeutic protein.

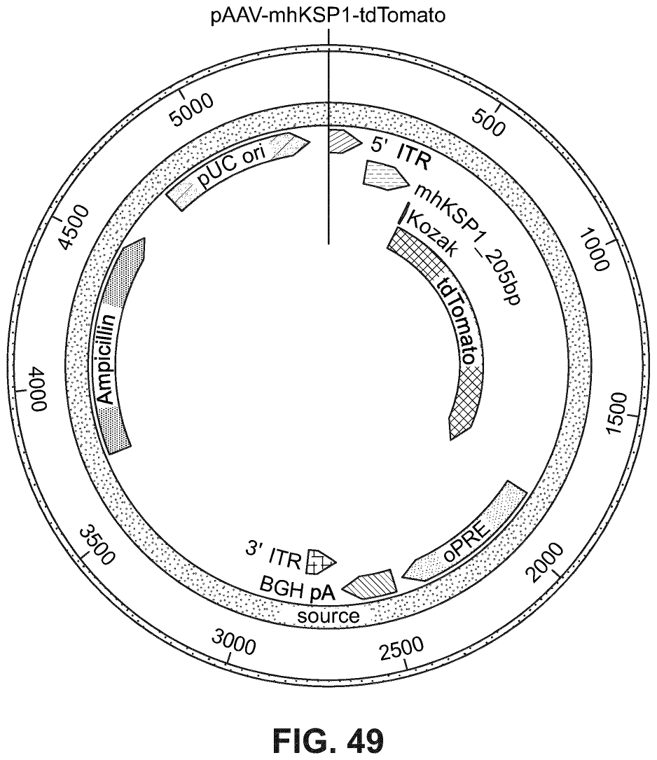

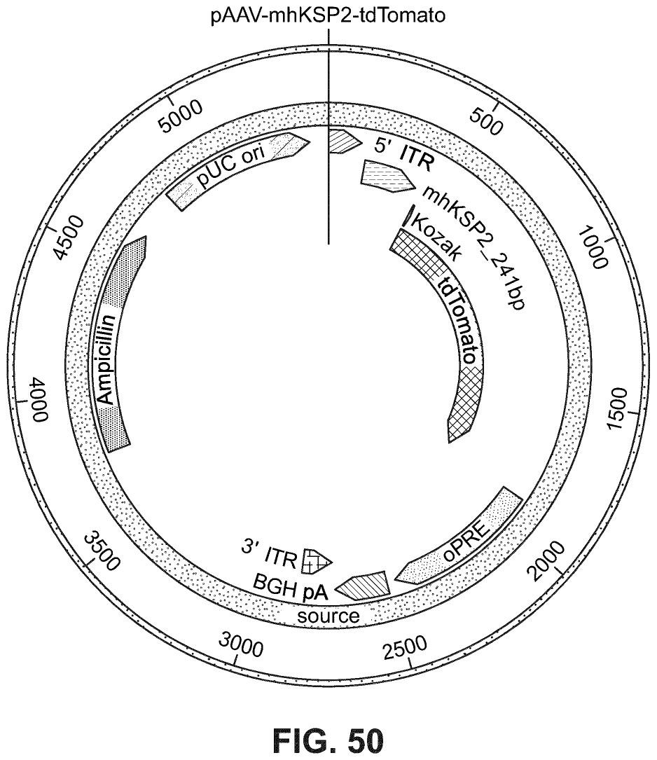

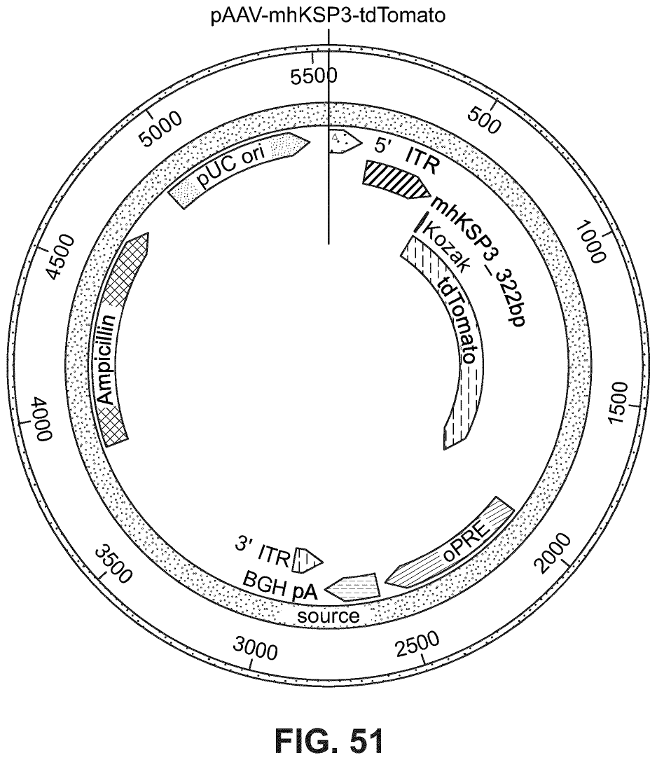

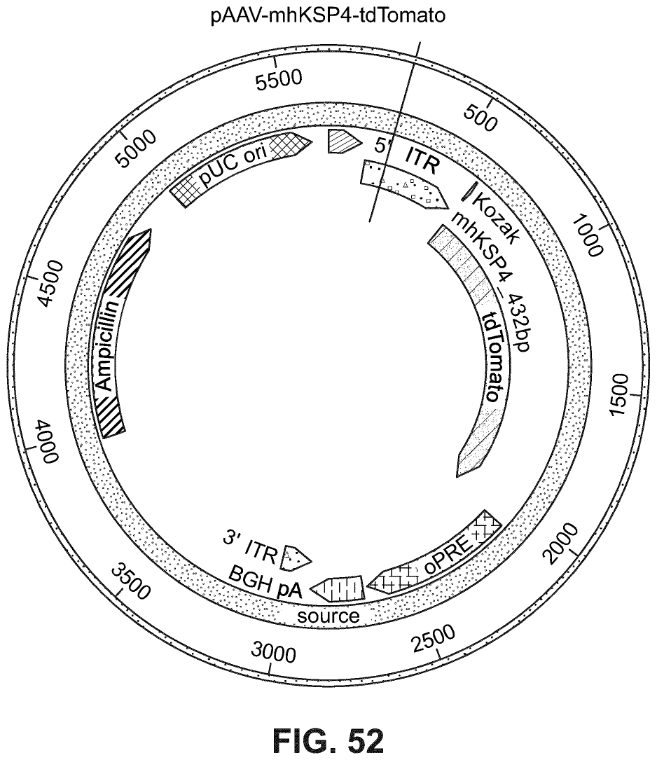

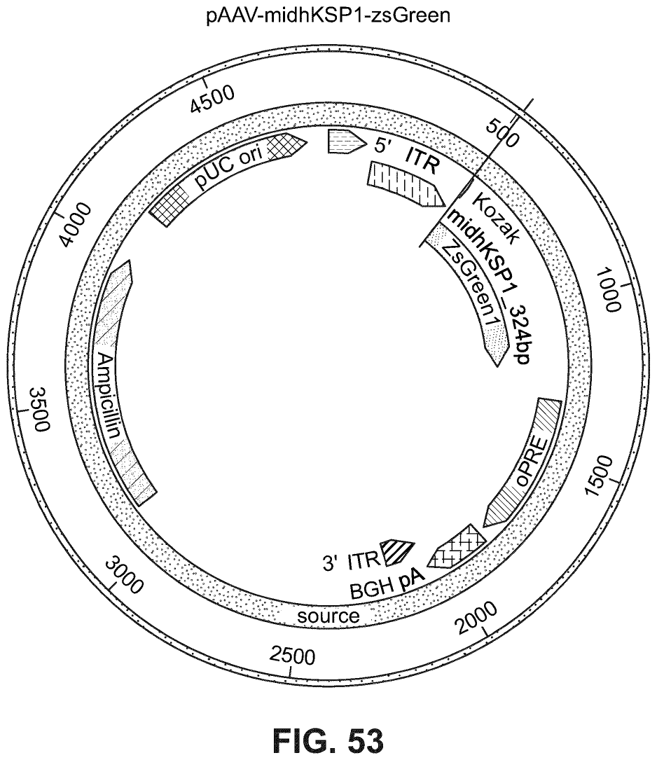

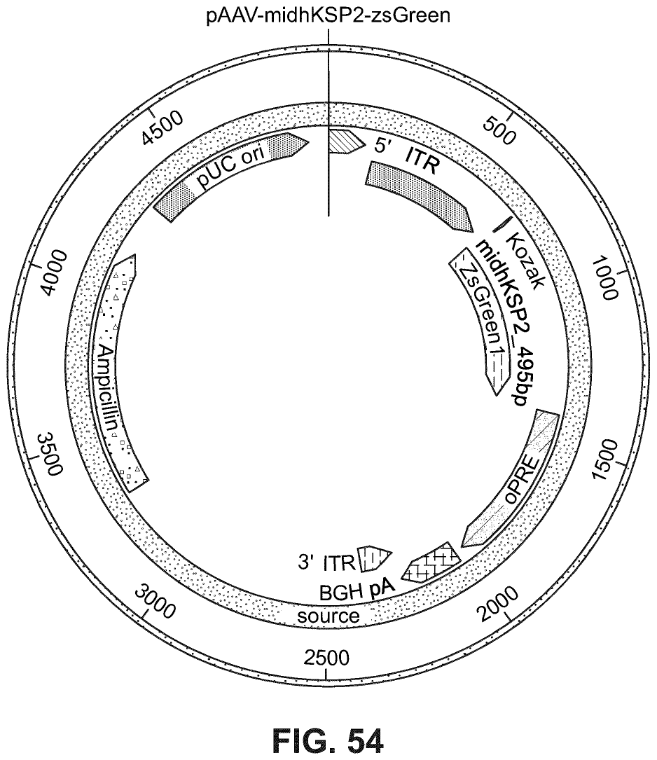

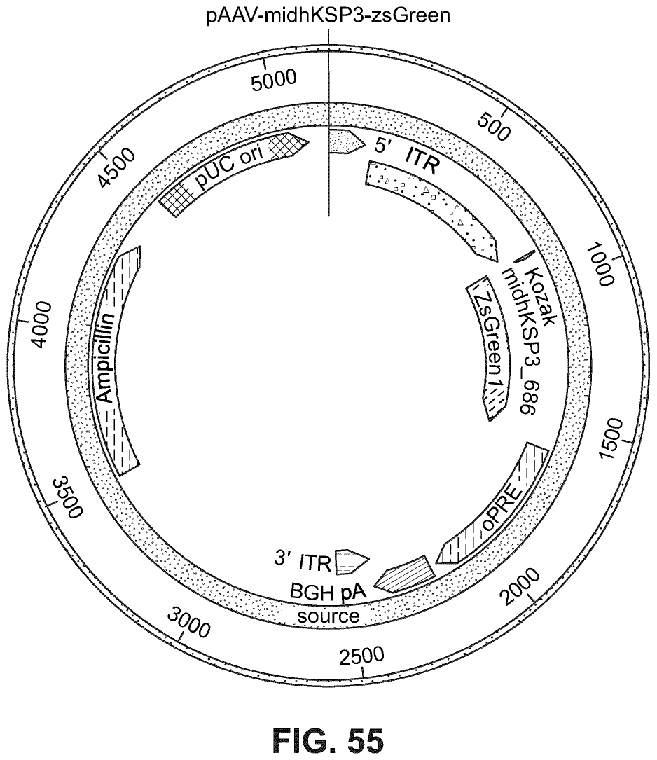

TABLE 6
Candidate construct

| Construct | Components | Sequence |
|---|---|---|
| pAAV-mhKSP1-tdTomato | 5′ ITR | SEQ ID NO: 32 |
| (FIG. 49) | mhKSP1 promoter | |
| | Reporter gene: tdTomato | |
| | oPRE | |
| | Terminator | |
| | 3′ ITR | |
| pAAV-mhKSP2-tdTomato | 5′ ITR | SEQ ID NO: 33 |
| (FIG. 50) | mhKSP2 promoter | |
| | Reporter gene: tdTomato | |
| | oPRE | |
| | Terminator | |
| | 3′ ITR | |
| pAAV-mhKSP3-tdTomato | 5′ ITR | SEQ ID NO: 34 |
| (FIG. 51) | mhKSP3 promoter | |
| | Reporter gene: tdTomato | |
| | oPRE | |
| | Terminator | |
| | 3′ ITR | |
| pAAV-mhKSP4-tdTomato | 5′ ITR | SEQ ID NO: 35 |
| (FIG. 52) | mhKSP4 promoter | |
| | Reporter gene: tdTomato | |
| | oPRE | |
| | Terminator | |
| | 3′ ITR | |
| pAAV-midhKSP1-zsGreen | 5′ ITR | SEQ ID NO: 36 |
| (FIG. 53) | midhKSP1 promoter | |
| | Reporter gene: zsGreen | |
| | oPRE | |
| | Terminator | |
| | 3′ ITR | |
| pAAV-midhKSP2-zsGreen | 5′ ITR | SEQ ID NO: 37 |
| (FIG. 54) | midhKSP2 promoter | |
| | Reporter gene: zsGreen | |
| | oPRE | |
| | Terminator | |
| | 3′ ITR | |
| pAAV-midhKSP3-zsGreen | 5′ ITR | SEQ ID NO: 38 |
| (FIG. 55) | midhKSP3 promoter | |
| | Reporter gene: zsGreen | |
| | oPRE | |
| | Terminator | |
| | 3′ ITR | |

### Example 11 (Prophetic): Cell-Based Screening for Identifying Therapeutic Constructs for ADPKD Caused by Mutations in PKD1

Cell-based assays can be used to identify constructs encoding PC1 CTF or CTT subdomains capable of providing sufficient levels of the respective PC1 subdomain to reduce or reverse cellular phenotypes observed in cells deficient in PC1. Initial screens can use transient transfection of plasmids encoding the therapeutic GOI and immortalized cell lines to provide a rapid, cost-effective approach to identifying potential therapeutic constructs. Subsequent experiments can be performed to vectorize the GOI into an AAV format, preferably as AAV5. However, the person of ordinary skill in the art will recognize a desire to potentially employ other native AAV serotypes or AAVs with engineered capsids for in vitro experiments. GOI constructs that effectively produce PC1 subdomains which correct for PC1 deficiency in immortalized cell lines can be evaluated in PC1 deficient organoids derived from hiPSCs.

### 11.1 Screening in Immortalized Cell Lines

In an illustrative example of an in vitro system, immortalized human, porcine, and canine kidney tubular epithelial cells (HK, LLC-PK1, and MDCK respectively) can be used to identify functional PC1-CTF and CTT encoding constructs. One of ordinary skill in the art will recognize that ADPKD is a disease of kidney tubular epithelial cells, so many cell lines representing the tubular regions of the nephron including the proximal tubule, loop of Henle, distal convoluted tubule, connecting tubule, or collecting duct (e.g., inner medullary collecting duct cells) may be used for screening PC1 encoding constructs. Further, as the function of polycystins is evolutionary conserved, cell lines derived from a range of species, including human, mouse, dog, pig, zebra fish, etc., are suitable for screening PC1 encoding constructs. In parallel, a PKD1 disease model can be generated by genome editing with CRISPR/Cas9 technology. Two genotypes will be obtained: (1) homozygous (PKD1^{-/-}) with complete absence of PC1 protein, and (2) heterozygous (PKD1^{+/-}) with reduced amount of PC1 protein. The homozygous PKD1^{-/-} cells, which are fully depleted of PC1 protein, can facilitate screening of therapeutic constructs based on PKD1 CTF and CTT mRNA and PC1 CTF and CTT protein expression and evaluation of target engagement. The heterozygous PKD1^{+/-} cell line carrying one PKD1 mutation, and therefore mimicking the human genetic ADPKD situation, can be used for further validation. Plasmids carrying the desired GOI can be designed as described in Example 10. GOIs carrying a tag, such as HA or FLAG, may be prepared in order to easily discriminate them from endogenous PKD1 mRNA/PC1 protein.

Transient transfection experiments can be performed in wild type and genome edited cell lines using a range of DNA amounts and transfection techniques appropriate for the cell line of interest. One of ordinary skill in the art will appreciate that each cell line may require use of a particular transfection reagent or approach to yield sufficient transfection levels for experimental evaluation. PKD1 CTF and CTT mRNA level can be assessed with a target specific set of primer/probes by RT-qPCR and compared to wild type levels (using PKD1 species-specific probes as needed). Protein level can be evaluated by Western blotting with antibodies directed against PC1 CTF or CTT or peptide tag and compared to wild type levels. Constructs that provide expression levels ±50% of wild type levels can be considered for subsequent functional analysis. As desired, experiments can be repeated using AAV encapsidated CTF and CTT encoding constructs. In such experiments, multiplicities of infection (MOIs) ranging from 10,000 to 1,000,000 can be tested.

Since PC1 is involved in proliferation, apoptosis, and cyst formation, as well as in Ca²⁺ signalling and metabolic pathways, target engagement in 2D models can be defined by impact/modification of GOIs on these pathways. The effect of PC1 CTF and CTT encoding constructs on proliferation can be evaluated with proliferation assays based on BrdU incorporation (or other more sensitive compounds such as IdU/CldU, EdU) (Abcam/Thermofisher). Upon expression of sufficient amounts of PC1 CTF or CTT, introduced by transfection or AAV-mediated transduction, the level of proliferation will be decreased as compared to the proliferation observed in the comparator PC1 deficient cells.

PC1 LoF causes increased apoptosis. The effect of PC1 CTF and CTT encoding constructs on apoptosis can be evaluated with apoptosis assays such as the Tunnel assay or by measuring levels of cleaved caspase-3. Upon expression of sufficient amounts of PC1 CTF or CTT, introduced by transfection or AAV-mediated transduction, the level of cleaved caspase-3 or signal in a Tunnel assay will be decreased as compared to the signal observed in the comparator PC1 deficient cells.

Since it has been shown in vitro that nitric oxide (NO) level is reduced in ADPKD cell lines, the effect of PC1 CTF and CTT encoding constructs on NO generation can be evaluated by flow cytometry with fluorescent NO indicator (DAF-2/DA). Upon expression of sufficient amounts of PC1 CTF or CTT, introduced by transfection or AAV-mediated transduction, the level of NO will be increased as compared to the NO levels observed in the comparator PC1 deficient cells.

Since it has been shown that polycystins regulates mitochondrial function and that loss of or reduced levels of PC1 lead to metabolic dysregulation as shown by reduced oxidative phosphorylation and increased glycolysis, the effect of PC1 CTF and CTT encoding constructs on oxidative phosphorylation can be evaluated with the Seahorse assay. Upon expression of sufficient amounts of PC1 CTF or CTT, introduced by transfection or AAV-mediated transduction, the level of oxidative phosphorylation will be increased, and the level of glycolysis will be decreased as compared to the levels observed in the comparator PC1 deficient cells.

MDCK cells are naturally deficient in PC1 and spontaneously form cysts under the appropriate culture conditions (Boletta et. Al., Molecular Cell, 2000, Vol. 6). MDCK cells can be used to investigate the capacity of PC1 CTF and CTT encoding constructs to revert/prevent the cyst growth as described in Boletta et al. Upon expression of sufficient amounts of PC1 CTF or CTT, introduced by transfection or AAV-mediated transduction, the level of spontaneous cyst formation by MDCK cells will be reduced or eliminated.

11.2 Screening in hiPSC-Derived ADPKD Kidney Organoids

PC1 CTF and CTT encoding constructs that effectively reduce or revert cellular phenotypes associated with loss of or reduced levels of PC1 in the assays described above can be further evaluated in PC1 deficient hiPSC-derived kidney organoids (Freedman et. al., Nature Communications, 2015, Vol. 6). These hiPSC-derived kidney organoids provide human tissue 3D models which are composed of podocytes, proximal and distal tubules, and associated endothelium and mesenchyme. PC1-deficient hiPSC-derived organoids mimic several of the phenotypes found in ADPKD patients, including metabolic dysregulation and cyst formation and growth.

Transient transfection experiments can be performed in wild type and genome edited hiPSCs using a range of DNA amounts and transfection techniques appropriate for the organoids. PKD1 CTF and CTT mRNA level can be assessed with target specific set of primer/probes by RT-qPCR and compared to wild type levels. Protein levels can be evaluated by Western blotting with antibodies directed against PC1 CTF or CTT or peptide tag and compared to wild type levels. Constructs that provide expression levels ±50% of wild type levels can be considered for subsequent functional analysis. As desired, experiments can be repeated using AAV encapsidated CTF and CTT encoding constructs. In such experiment, MOIs ranging from 10,000 to 1,000,000 can be tested.

Since it has been shown that polycystins regulates mitochondrial function and that loss of or reduced levels of PC1 lead to metabolic dysregulation as shown by reduced oxidative phosphorylation and increased glycolysis, the effect of PC1-CTF and CTT encoding constructs on oxidative phosphorylation can be evaluated with the Seahorse assay. Upon expression of sufficient amounts of PC1 CTF or CTT in PC1 deficient hiPSC-derived kidney organoids, introduced by transfection or AAV-mediated transduction, the level of oxidative phosphorylation will be increased, and level of glycolysis will be decreased as compared to the levels observed in the comparator PC1 deficient organoids.

PC1-deficient hiPSC-derived kidney organoid spontaneously form cysts, which expand over time in culture (Freedman et. al., Nature Communications, 2015, Vol. 6; Cruz et. al., Nature Materials, 2017, Vol. 16), the effect of PC1-CTF and CTT encoding constructs on spontaneous cystogenesis will be evaluated by scoring cyst number, volume, and other metrics of cyst formation. Upon expression of sufficient amounts of PC1 CTF or CTT in PC1 deficient hiPSC-derived kidney organoids, introduced by transfection or AAV-mediated transduction, the number and volume of cysts formed will be decreased as compared to the levels observed in the comparator PC1 deficient cells. Depending on the efficiency of transfection or transduction, markers may be introduced in PC1-encoding constructs to identify transfected/transduced cells for analysis. In this case, comparison of cyst number and volume in transfected/transduced versus non-transfected/transduced cells can also be assessed.

### Example 12 (Prophetic): Cell-Based Screens for Identifying Therapeutic Constructs for ADPKD Caused by Mutations in PKD2

Cell-based assays can be used to identify constructs encoding PC2 capable of providing sufficient levels of PC2 to reduce or reverse cellular phenotypes observed in cells deficient in PC2. Initial screens can use transient transfection of plasmids encoding the therapeutic GOIs and immortalized cell lines to provide a rapid, cost-effective means of identifying potential therapeutic constructs. Subsequent experiments will vectorize the GOI into an AAV format, preferably as AAV5. However, one of ordinary skill in the art will recognize a desire to potentially employ other native AAV serotypes or AAVs with engineered capsids for in vitro experiments. GOI constructs that effectively produce PC2, which correct for PC2 deficiency in immortalized cell lines, can be evaluated in PC1 deficient organoids derived from hiPSCs.

### 12.1 Screening in Immortalized Cell Lines

In an illustrative example of an in vitro system, immortalized human, porcine, and canine kidney tubular epithelial cells (HK, LLC-PK1 and MDCK respectively) can be used to identify functional PC2-encoding constructs. One of ordinary skill in the art will recognize that ADPKD is a disease of kidney tubular epithelial cells, so many cell lines representing the tubular regions of the nephron including the proximal tubule, loop of Henle, distal convoluted tubule, connecting tubule, or collecting duct (e.g., inner medullary collecting duct cells) may be used for screening PC2-encoding constructs. Further, as the function of polycystins is evolutionary conserved, cell lines derived from a range of species, including human, mouse, dog, pig, zebra fish, etc., are suitable screening PC2 encoding constructs. In parallel, a PKD2 disease model can be generated by genome editing with CRISPR/Cas9 technology. Two genotypes will be obtained: (1) homozygous (PKD2^{-/-}) with complete absence of PC2 protein, and (2) heterozygous (PKD2^{+/-}) with reduced amount of PC2 protein. The homozygous PKD2^{-/-} cells, which are fully depleted of PC2 protein, can facilitate screening of therapeutic construct based on PKD2 mRNA and PC2 protein expression and evaluation of target engagement. The heterozygous PKD2^{+/-} cell line carrying one PKD2 mutation, and therefore mimicking the human genetic ADPKD situation, can be used for further validation. Plasmids carrying the desired GOI will be design as described in Example 10. GOIs carrying a tag such as HA or FLAG may be prepared in order to easily discriminate them from endogenous PKD2 mRNA/PC2 protein.

Transient transfection experiments can be performed in wild type and genome edited cell lines using a range of DNA amounts and transfection techniques appropriate for the cell line of interest. One of ordinary skill in the art will appreciate that each cell line may require use of a particular transfection reagent or approach to yield sufficient transfection levels for experimental evaluation. PKD2 mRNA level can be assessed with a target specific set of primer/probes by RT-qPCR and compared to wild type levels (using PKD2 species specific probes as needed). Protein level can be evaluated by Western blotting with antibodies directed against PC2 (sc-28331) or tag (H3663, F7425) and compared to wild type levels. Constructs that provide expression levels ±50% of wild type levels can be considered for subsequent functional analysis. As desired, experiments can be repeated using AAV encapsidated PC2-encoding constructs. In such experiments, MOIs ranging from 10,000 to 1,000,000 can be tested.

Since PC2 interacts with PC1 and is involved in PC1 maturation, apoptosis, metabolic regulation, cyst formation, and Ca²⁺ signalling, target engagement in 2D models can be defined by impact/modification of GOIs on these pathways. In the absence of PC2, PC1 processing (e.g., cleavage at the GPS) and transit from the ER is impaired. The effect of PC2-encoding constructs on PC1 maturation and transport can be evaluated by Western blotting to measure PC1 expression and IF to monitor the cellular localization of PC1. Upon expression of sufficient amounts of PC2, introduced by transfection or AAV-mediated transduction, the level of PC1 will be increased as compared to PC1 levels observed in the comparator PC2 deficient cells. PC1 present will include full length, as well as PC1 cleaved at the GPS to generate the NTF and CTF subdomains of PC1. Additionally, in cells expressing sufficient amounts of PC2, PC1 will be present in the plasma membrane, and in cilium if present, and cleavage products of PC1 such as the CTT may also be present in the nucleus or mitochondria.

PC2 LoF causes increased apoptosis. The effect of PC2-encoding constructs on apoptosis will be evaluated with apoptosis assays such as the Tunnel assay or by measuring levels of cleaved caspase-3. Upon expression of sufficient amounts of PC2, introduced by transfection or AAV-mediated transduction, the level of cleaved caspase-3 or signal in a Tunnel assay will be decreased as compared to the signal observed in the comparator PC2 deficient cells.

Since it has been shown that polycystins regulates mitochondrial function and that loss of or reduced levels of PC2 lead to metabolic dysregulation as shown by reduced oxidative phosphorylation and increased glycolysis, the effect of PC2-encoding constructs on oxidative phosphorylation can be evaluated with the Seahorse assay. Upon expression of sufficient amounts of PC2, introduced by transfection or AAV-mediated transduction, the level of oxidative phosphorylation will be increased, and the level of glycolysis will be decreased as compared to the levels observed in the comparator PC2 deficient cells.

PC2 functions as a Ca²⁺ permeable nonselective cation channel, homologous to the transient receptor potential family of cation channels. Loss of or reduced PC2 levels lead to dysregulation of Ca²⁺, leading to an imbalance in Ca²⁺ in ER. The effect of PC2-encoding constructs on Ca²⁺ in the ER can be assessed with a fluorometric-based assay using a calcium indicator. Upon expression of sufficient amounts of PC2, introduced by transfection or AAV-mediated transduction, the level of Ca²⁺ in the ER will be decreased as compared to the levels observed in the comparator PC2 deficient cells.

12.2 Screening in hiPSC-Derived ADPKD Kidney Organoids

PC2-encoding constructs that effectively reduce or revert cellular phenotypes associated with loss of or reduced levels of PC2 in the assays described above can be further evaluated in PC2 deficient hiPSC-derived kidney organoids (Freedman et al.) These hiPSC-derived kidney organoids provide human tissue 3D models which are composed of podocytes, proximal, and distal tubules and associated endothelium and mesenchyme. PC2-deficient hiPSC-derived organoids mimic several of the phenotypes found in ADPKD patients, including metabolic dysregulation and cyst formation and growth.

Transient transfection experiments can be performed in wild type and genome edited hiPSCs using a range of DNA amounts and transfection techniques appropriate for the organoids. PKD2 mRNA level can be assessed with target specific set of primer/probes by RT-qPCR and compared to wild type levels. Protein levels can be evaluated by Western blotting with antibodies directed against PC2 or peptide tag and compared to wild type levels. Constructs that provide expression levels ±50% of wild type levels can be considered for subsequent functional analysis. As desired, experiments can be repeated using AAV encapsidated PC2-encoding constructs. In such experiment, MOIs ranging from 10,000 to 1,000,000 can be tested.

Since PC2 interacts with PC1 and is involved in PC1 maturation, the effect of PC2-encoding constructs on PC1 maturation and transport can be evaluated by Western blotting to measure PC1 expression and IF to monitor the cellular localization of PC1. Upon expression of sufficient amounts of PC2 in PC2 deficient hiPSC-derived kidney organoids, introduced by transfection or AAV-mediated transduction, the level of PC1 will be increased as compared to PC1 levels observed in the comparator PC2 deficient organoids. PC1 present will include full length, as well as PC1 cleaved at the GPS to generate the NTF and CTF of PC1. Additionally, in cells expressing sufficient amounts of PC2, PC1 will be present in the plasma membrane, and in cilium if present, and cleavage products of PC1 such as the CTT may also be present in the nucleus or mitochondria.

Since it has been shown that polycystins regulates mitochondrial function and that loss of or reduced levels of PC2 lead to metabolic dysregulation as shown by reduced oxidative phosphorylation and increased glycolysis, the effect of PC2-encoding constructs on oxidative phosphorylation can be evaluated with the Seahorse assay. Upon expression of sufficient amounts of PC2 in PC2 deficient hiPSC-derived kidney organoids, introduced by transfection or AAV-mediated transduction, the level of oxidative phosphorylation will be increased, and level of glycolysis will be decreased as compared to the levels observed in the comparator PC2 deficient organoids.

Since loss of or reduced PC2 levels lead to dysregulation of Ca²⁺, leading to an imbalance in Ca²⁺ in ER, the effect of PC2-encoding constructs on Ca²⁺ in the ER can be assessed with a fluorometric-based assay using a calcium indicator. Upon expression of sufficient amounts of PC2 in PC2 deficient hiPSC-derived kidney organoids, introduced by transfection or AAV-mediated transduction, the level of Ca²⁺ in the ER will be decreased as compared to the levels observed in the comparator PC2 deficient organoids.

PC2-deficient hiPSC-derived kidney organoid spontaneously form cysts, which expand over time in culture (Freedman et. al., Nature Communications, 2015, Vol. 6; Cruz et. al., Nature Materials, 2017, Vol. 16), the effect of PC2-encoding constructs on spontaneous cystogenesis can be evaluated by scoring cyst number, volume and other metrics of cyst formation. Upon expression of sufficient amounts of PC2 in PC2 deficient hiPSC-derived kidney organoids, introduced by transfection or AAV-mediated transduction, the number and volume of cysts formed will be decreased as compared to the levels observed in the comparator PC2 deficient cells. Depending on the efficiency of transfection or transduction, markers may be introduced in PC2-encoding constructs to identify transfected/transduced cells for analysis. In this case, comparison of cyst number and volume in transfected/transduced versus non-transfected/transduced cells can also be assessed.

### Example 13 (Prophetic): Evaluation of ADPKD Gene Tx in Mouse Models of ADPKD

Constructs which demonstrate expression levels ±50% of endogenous PC1 or PC2, molecular evidence of pathway engagement, and evidence of rescue or prevention of cellular phenotypes consistent with ADPKD pathology in immortalized 2D and 3D and iPSC derived 2D cell and 3D organoid models as described in Examples 11 and 12 can be further evaluated in mouse models of ADPKD as follows.

### 13.1 AAV Serotype and Dose

Multiple AAVs can be used to determine the most potent AAV for kidney targeting. This includes all AAV main serotypes from AAV1 to AAV13 and AAV peptide display libraries of the aforementioned parental serotypes which can be used here to determine lead AAV candidates in terms of kidney tissue specificity and transduction efficiency. Preferably, AAV5 will be considered as one of the main serotypes used based on the study described in Example 4. High and low dose of vectors encapsidating therapeutic constructs and reporter genes can be administered in the mouse models ranging from 1×10¹⁰ to 2×10¹⁴ vector genomes (vgs) per mouse. It is contemplated that the preferred AAV for transduction of the kidney may differ by route of administration and that the use of surrogate serotypes (i.e., a serotype different than the preferred serotype for human therapy delivered via LRP) may be necessary to evaluate therapeutic AAV constructs in mice.

### 13.2 AAV Route of Administration

Mice will be followed up from 1 week up to 24 weeks post administration. Phenotypic analyses as well as molecular characterization of the therapeutic constructs will be evaluated at each time point during the study. Multiple routes of administration can be investigated for kidney tissue and renal cell transduction including systemic and direct kidney delivery. Tail vein, facial vein, femoral vein, retro-orbital, jugular vein, and intraperitoneal injection can be used to administer AAVs systemically. For direct delivery to the kidney, renal artery, renal vein, intraparenchymal, and ureter injections (i.e., retro-ureteral route) can be performed. Clamping from 5 min to 45 min post renal vein injection may be used to enhance transduction.

Routes of AAV delivery to kidney are summarized in Davis and Park et al., “Gene therapy research for kidney diseases,” Physiol. Genomics 2019 Vol. 51 (9), 449-461.

### Example 14 (Prophetic): Evaluation of Therapeutic AAV Constructs for the Treatment of PKD1 ADPKD

This example employs a conditional PKD1-KO mouse model of ADPKD (Pkd1^{fl/fl}; Pax8^{rtTA}, Tet-Cre) (Ma et. al., Nature Genetics 2013, Vol. 45). Inactivation of PC1 expression was induced with 2 mg/ml doxycycline in drinking water supplemented with 3% sucrose for 2 weeks from P28 to P42. This resulted in fully penetrant loss of PC1 expression in targeted renal epithelial cells and the development of polycystic kidney disease (PKD) as assessed by kidney weight/body weight % (kw/bw %); blood urea nitrogen levels (BUN), serum creatinine, MRI measurement of kidney volume, and histologic evaluation of the kidneys post-sacrifice. Disease onset is progressive with substantially enlarged kidneys and concomitant loss of kidney function observed by 13 weeks post conditional knockout of PC1, with continuing progression through 16 and 24 weeks. For these experiments, group sizes will be 12-15 mice per group and the effect of treatment with the gene therapy assessed at various timepoints.

### 14.1 Evaluation in Preventative Models of ADPKD

For evaluation of constructs in preventative models of disease, PC1 CTF or CTT encoding AAV constructs can be administered by retro-orbital injection to P1 pups at a total dose ranging from 1×10¹⁰ to 2×10¹⁴ vgs per mouse. Littermates untreated with doxycycline can serve as a positive control (e.g., wild type mice), and doxycycline-induced non-AAV treated littermates can serve as the comparator. Multiples of each group can be included in the study to allow for assessment at 10, 13, 16 and 19 weeks of age. In-life assessments include measurement of BUN, serum creatinine, and kidney volume by MRI. Post-sacrifice assessments include kw/bw % and histologic evaluation of the kidneys. Expression of CTF or CTT within the kidneys by transduction with AAV coding for the respective transgene is anticipated to effectively slow or prevent the progression of ADPKD following conditional knock-out of PKD1, as demonstrated by a reduction in kw/bw %, BUN level, serum creatinine level, and kidney volume in treated versus untreated mice. The effect is dose-dependent with results at the highest dose of AAV administered approaching values observed in wild type mice. Differences between treatment and control groups are expected to be negligible at 10 weeks, and evident at 13, 16, and 19 weeks. Additionally, kidneys from treated mice are expected to contain substantially fewer and smaller cysts than untreated mice as assessed by histology.

### 14.2 Evaluation in Treatment Models of ADPKD

For evaluation of constructs in treatment models of disease, PC1 CTF or CTT encoding AAV constructs can be administered by the preferred systemic route or by direct delivery to the kidney at 10 weeks at a total dose ranging from 1×10¹⁰ to 2×10¹⁴ vgs per mouse. It is contemplated that the route of administration may vary with the AAV serotype used, and that administration at week 10 will enable expression of the CTF or CTT protein by week 13. Littermates untreated with doxycycline can serve as a positive control (e.g., wild type mice), and doxycycline-induced non-AAV treated littermates can serve as the comparator. Multiples of each group can be included in the study to allow for assessment at 13, 16 and 19 weeks of age. In-life assessments include measurement of BUN, serum creatinine, and kidney volume by MRI. Post-sacrifice assessments include kw/bw % and histologic evaluation of the kidneys. Expression of CTF or CTT within the kidneys by transduction with AAV coding for the respective transgene is anticipated to effectively slow the progression of or reverse PKD following conditional knock-out of PKD1, as demonstrated by a reduction in kw/bw %, BUN level, serum creatinine level, and kidney volume in treated versus untreated mice. The effect is dose-dependent with results at the highest dose of AAV administered approaching values observed in wild type mice. Differences between treatment and control groups are expected to be evident at 13, 16 and 19 weeks. Additionally, kidneys from treated mice are expected to contain substantially fewer and smaller cysts than untreated mice as assessed by histology.

In both the preventative and treatment models, biodistribution assessments of transduction and transgene expression can be conducted as follows.

### 14.3 Quantification of Vector Genomes and Biodistribution

Total genomic DNA can be extracted from mouse tissue samples after sacrifice. Number of vector genomes in cells can be evaluated using ddPCR. Vector genomes can be detected using a designed primer probe set against CTT, CTF, or AAV ITRs and can be measured against a housekeeping gene (i.e., primer probe set for Beta Actin).

Quantifying vector genomes in kidney in combination with phenotypic analysis can allow for determination of the dose range needed to achieve a therapeutic effect.

14.4 Expression Analysis by mRNA Relative Quantification and Immunoblotting

Total RNA can be isolated from kidney tissues. RT-qPCR can be used to determine relative concentration of CTT or CTF encoding mRNA against a house keeping gene (PolR2A or GAPDH) using a specific primer probe set.

Total proteins can be isolated from homogenized and lysed tissue and Western blotting can be performed using recommended dilutions of anti-CTT, anti-CTF, anti-HA monoclonal antibodies.

Increase in specific mRNA expressed from the corresponding constructs is anticipated. Full size of protein of CTT or CTF with or without HA tag is also anticipated in treated tissue samples.

### Example 15 (Prophetic): Evaluation of Therapeutic AAV Constructs for the Treatment of PKD2 ADPKD

This example employs a conditional pkd2-KO mouse model of ADPKD (Pkd2^{fl/fl}; Pax8^{rtTA}, Tet-Cre) (Ma et. al., Nature Genetics 2013, Vol. 45). Inactivation of PC2 expression was induced with 2 mg/ml doxycycline in drinking water supplemented with 3% sucrose for 2 weeks from P28 to P42. This results in fully penetrant loss of PC2 expression in targeted renal epithelial cells and the development of polycystic kidney disease as assessed by kidney weight/body weight % (kw/bw %); blood urea nitrogen levels (BUN), serum creatinine, MRI measurement of kidney volume, and histologic evaluation of the kidneys post-sacrifice. Disease onset is progressive with substantially enlarged kidneys and concomitant loss of kidney function observed by 13 weeks post conditional knockout of PC2, with continuing progression through 16 and 24 weeks. For these experiments, group sizes are 12-15 mice per group and the effect of treatment with the gene therapy assessed at various time points.

### 15.1 Evaluation in Preventative Models of ADPKD

For evaluation of constructs in preventative models of disease, PC2 or PC1 CTF or CTT encoding AAV constructs can be administered by retro-orbital injection to P1 pups at a total dose ranging from 1×10¹⁰ to 2×10¹⁴ vgs per mouse. Littermates untreated with doxycycline can serve as a positive control (e.g., wild type mice), and doxycycline-induced non-AAV treated littermates can serve as the comparator. Multiples of each group can be included in the study to allow for assessment at 10, 13, 16 and 19 weeks of age. In-life assessments include measurement of BUN, serum creatinine, and kidney volume by MRI. Post-sacrifice assessments include kw/bw % and histologic evaluation of the kidneys. Expression of PC2 or PC1 CTF or CTT within the kidneys by transduction with AAV coding for the respective transgene is anticipated to effectively slow or prevent the progression of ADPKD following conditional knock-out of PKD2, as demonstrated by a reduction in kw/bw %, BUN level, serum creatinine level, and kidney volume in treated versus untreated mice. The effect is dose-dependent with results at the highest dose of AAV administered approaching values observed in wild type mice. Differences between treatment and control groups are expected to be negligible at 10 weeks, and evident at 13, 16 and 19 weeks. Additionally, kidneys from treated mice are expected to contain substantially fewer and smaller cysts than untreated mice as assessed by histology.

### 15.2 Evaluation in Treatment Models of ADPKD

For evaluation of constructs in treatment models of disease, PC2 or PC1 CTF or CTT encoding AAV constructs can be administered by the preferred systemic route or by direct delivery to the kidney at 10 weeks at a total dose ranging from 1×10¹⁰ to 2×10¹⁴ vgs per mouse. It is contemplated that the route of administration may vary with the AAV serotype used, and that administration at week 10 will enable expression of PC2 or PC1 CTF or CTT protein by week 13 pups. Littermates untreated with doxycycline can serve as a positive control (e.g., wild type mice), and doxycycline-induced non-AAV treated littermates can serve as the comparator. Multiples of each group can be included in the study to allow for assessment at 13, 16 and 19 weeks of age. In-life assessments include measurement of BUN, serum creatinine, and kidney volume by MRI. Post-sacrifice assessments include kw/bw % and histologic evaluation of the kidneys. Expression of PC2 or PC1 CTF or CTT within the kidneys by transduction with AAV coding for the respective transgene is anticipated to effectively slow the progression of or reverse PKD following conditional knock-out of PKD2, as demonstrated by a reduction in kw/bw %, BUN level, serum creatinine level, and kidney volume in treated versus untreated mice. The effect is dose-dependent with results at the highest dose of AAV administered approaching values observed in wild type mice. Differences between treatment and control groups are expected to be evident at 13, 16 and 19 weeks. Additionally, kidneys from treated mice are expected to contain substantially fewer and smaller cysts than untreated mice as assessed by histology.

In both the preventative and treatment models, biodistribution assessments of transduction and transgene expression can be conducted as follows.

### 15.3 Quantification of Vector Genomes and Biodistribution

Total genomic DNA can be extracted from mouse tissue samples after sacrifice. Number of vector genomes in cells can be evaluated using ddPCR. Vector genomes can be detected using a designed primer probe set against PKD2, PKD1 CTT, PKD1 CTF, or AAV ITRs and can be measured against a housekeeping gene (i.e., primer probe set for Beta Actin).

Quantifying vector genomes in kidney in combination with phenotypic analysis can allow for determination of the dose range needed to achieve a therapeutic effect.

15.4 Expression Analysis by mRNA Relative Quantification and Immunoblotting

Total RNA can be isolated from kidney tissues. RT-qPCR can be used to determine relative concentration of PC2 or PC1 CTT or CTF-encoding mRNA against a house keeping gene (PolR2A or GAPDH) using a specific primer probe set.

Total proteins can be isolated from homogenized and lysed tissue and Western blotting can be performed using recommended dilutions of anti-PC2, anti-CTT, anti-CTF, and anti-HA monoclonal antibodies.

Increase in specific mRNA expressed from the corresponding constructs is anticipated. Full size of protein of PC2 or PC1 CTT or CTF with or without HA tag is also anticipated in treated tissue samples.

### Example 16 (Prophetic): Evaluating of ADPKD Gene Therapeutic AAV Constructs in Healthy and ADPKD Diseased Pigs

Constructs which demonstrate expression levels ±50% of endogenous PC1 or PC2, molecular evidence of pathway engagement, or evidence of rescue or prevention of cellular phenotypes consistent with ADPKD pathology as described in Examples 11 and 12, and which may also prevent, slow, or revert disease progression in mouse models of ADPKD as described in Examples 13-15, can be further evaluated in pigs as follows.

Pigs can be used to evaluate the safety of LRP in combination with ADPKD gene therapy to support evaluation of exemplary embodiments in first-in-human clinical studies. Therefore, potential therapeutic constructs can be evaluated in the pig to identify those suitable for IND-enabling studies, and further development for ADPKD caused by mutations in the PKD1 or PKD2 genes.

For constructs intended to treat ADPKD caused by mutations in the PKD1 gene, additional studies may be conducted in the PKD1 deficient pig model to evaluate efficacy of the combination product in a large animal model of disease.

### 16.1 Evaluations of PKD1 and PKD2 AAV Constructs in Healthy Pigs

For constructs intended to treat ADPKD caused by mutations in PKD1, the purpose of this example is to evaluate AAV gene therapeutic dose versus transduction efficiency and expression when delivered via kidney LRP to (1) aid in selection of development candidates, (2) determine dose ranges for potential evaluation in nonclinical safety studies, (3) determine dose ranges for potential evaluation in the pig model of PKD1 deficient ADPKD, and (4) determine dose ranges for potential evaluation in humans.

For constructs intended to treat ADPKD caused by mutations in PKD2, the purpose of this example is to evaluate AAV gene therapeutic dose versus transduction efficiency and expression when delivered via kidney LRP to (1) aid in selection of development candidates, (2) determine dose ranges for potential evaluation nonclinical safety studies, and (3) determine dose ranges for potential evaluation in humans.

Healthy pigs such as Sus scrofa domesticus, adult Yucatan minipigs, or other pigs weighing from 50-90 kg to enable use of LRP catheter components suitable in size for use in humans can be used in these studies (i.e., especially arterial supply and venous return perfusion catheters).

### 16.2 AAV Serotype and Dose

A range of AAV doses for PKD1 CTF and CTT encoding and PKD2-encoding constructs can be administered in healthy pigs ranging from 1×10¹⁰ vg to 2×10¹⁵ vg total. Multiple AAV variants can be tested including main serotypes AAV1 to AAV13, preferably AAV5 as this has been experimentally demonstrated to be efficient for LRP-mediated gene delivery to the kidney in pigs (see Example 4), and to provide transduction of the variety of cells in which cysts develop in ADPKD. Moreover, other synthetic AAVs engineered for kidney delivery or demonstrated by us and others to be efficient vectors in kidney can be tested for efficient kidney transduction.

It is contemplated that this set of experiments will enable the determination of a highly efficient or most efficient construct and AAV variant based on transduction and expression data, which can be addressed by the studies described below. Effective vector, dose, and construct combinations are anticipated to provide sufficient expression of PC1 CTF or CTT or PC2, and in the case of PC1-CTF or CTT, to alleviate symptoms and achieve a therapeutic benefit in the PKD1 pig model.

### 16.3 Treatment

Pigs for this study can be pre-selected based on (1) immunological screening for the AAV variant used in the study, and (2) CT scans to determine the optimal physiological compatibility with the LRP procedure. PKD1 CTF or CTT encoding and PKD2-encoding constructs encapsidated in AAV5 as described in Example 10, or in alternative AAV serotypes suitable for administration by kidney LRP, can be administered to one or more pigs as described in Example 5. Animals will be followed for two to sixteen weeks post administration. In-life assessments, which may include blood chemistry parameters, measures of kidney injury biomarkers, and expression of PC1-CTF, CTT or PC2 in urinary exosomes can be assessed periodically, as described below. Post sacrifice assessments, which may include quantification of biodistribution, histological evaluation for safety and cell specific transgenic protein expression, and single-cell RNASeq and ATAC-seq to quantify level of expression and proportion of target cells transduced can be assessed as described below.

### 16.4 Blood Chemistry Parameters

Blood samples can be collected to assess the concentrations of blood creatinine (CRE), urea nitrogen (BUN), aspartate aminotransferase (AST), alanine aminotransferase (ALT), and lactate dehydrogenase, among other parameters.

General blood parameters can be assessed and compared to untreated animals when constructs are administered to healthy pigs. When PKD1 CTF and CTT constructs are administered to PKD1 disease pigs, general blood parameters can be assessed compared to healthy wild-type animals and improvement is contemplated in measurements of kidney function (e.g., BUN, serum creatinine) for treated versus untreated animals.

### 16.5 Kidney Injury Markers in Serum and Urine Samples

Kidney injury markers such as kidney injury molecule-1 (KIM-1), cystatin C (Cys C), and neutrophil gelatinase-associated lipocalin (NGAL) can be measured using ELISA kits.

No change in these biomarkers is expected with administration of PKD1 or PKD2 therapeutic constructs to healthy pigs. When PKD1 CTF or CTT therapeutic constructs are administered to PKD1 disease pigs, lower amounts of injury markers may be expected in treated versus untreated diseased animals.

### 16.6 Monitoring of PC1 and PC2 Urinary Exosomes as Biomarkers for Transgene Expression

Hogan et al., “Identification of Biomarkers for PKD1 Using Urinary Exosomes,” J. Am. Soc. Nephrol. 2015, 26(7), 1661-70, described a test for measuring the urine exosomal PC1/TMEM2 or PC2/TMEM2 ratio for diagnosing and monitoring polycystic kidney disease.

Urine samples can be collected from all pigs before and after administration of payload that can be followed until sacrifice. Crude exosomes can be isolated by one-step ultracentrifugation. Additionally, specific urinary exosome-like vesicles (ELVs) that are known to contain PC1 or PC2 can be isolated by running a 5-30% sucrose D₂O gradient. SDS-PAGE, slice recovery, and MS/MS label-free proteomics can be used to determine peptide intensity in samples collected in addition to quantitative Western blotting using specific probes for PC1, PC2, and TMEM2 as described in the aforementioned study.

Therapeutic constructs can be identified based on their ability to provide a dose-dependent increase in PC1 and PC2/TMEM2 ratios in healthy pigs. In PKD1 disease pigs, effective constructs is contemplated to demonstrate an increase in PC1/TMEM and/or PC2/TMEM as compared to untreated animals.

### 16.7 Quantification of Vector Genomes and Biodistribution

Total genomic DNA can be extracted from pig tissue samples after sacrifice. The number of vector genomes in cells can be evaluated using ddPCR. Vector genomes can be detected using a designed primer probe set against PKD1 CTF or CTT, PKD2, or AAV ITRs and can be measured against a housekeeping gene (i.e., primer probe set for Beta Actin).

Quantifying vector genomes in the kidneys of pigs is expected to enable determination of the adequate dose to achieve a therapeutic benefit.

16.8 Expression Analysis by mRNA Relative Quantification and Immunoblotting

Total RNA can be isolated from kidney tissues. RT-qPCR can be used to determine relative concentration of PKD1 mRNA encoding CTF or CTT, or PKD2 mRNA, and against a house keeping gene (PolR2A or GAPDH) using a specific primer probe set.

Total proteins can be isolated from homogenized and lysed tissue and Western blot or ELISA assays can be performed using recommended dilutions of anti-PC1 CTF, anti-PCI CTT, anti-PC2, and anti-HA monoclonal antibodies.

Increase in specific mRNA expressed from the corresponding constructs is expected. It contemplated that full-size PC1 CTT or CTF protein with or without HA tag, or PC2 protein can be expected in treated tissue samples. Increase in PC1 CTT and CTF protein is expected in the PKD1 models.

16.9 scRNAs-Seq and scATAC-Seq Analysis

Single-cell RNA sequencing (scRNAseq) and single-cell sequencing assay for transposase-accessible chromatin (scATAC-seq) can be performed on both treated and untreated pig kidney tissues. Nuclear isolation can be performed on freshly snap frozen kidney tissues. Here, cryopreserved tissue is dissociated into a single-cell suspension and subsequently cell count and viability are determined to ensure sufficient number of cells/nuclei with acceptable cell viability (preferably ≥70%). Dead cells are to be removed before library preparation. Library generation for each of the sequencing methods and dead cell removal can be performed using specific kits such as those recommended by 10× Genomics® and others. Data can be processed and analyzed using 10× Genomics® Cell Ranger™ and Loupe™ Cell Browser.

The generated set of data can allow for obtaining comparable clustering of treated and untreated kidney tissues. Each cluster can be determined based on differential gene expression of a subset of cells. Therefore, this will allow for identification of precise cell targets transduced by exemplary vectors and follow up any transcriptomic changes triggered by the exemplary constructs.

It is contemplated that these analyses will demonstrate expression of PKD1 mRNA encoding CTF or CTT or PC2 mRNA in ≥25% of renal tubular epithelial cells that would be sufficient to meaningfully slow cyst progression in a human or animal deficient in PC1 or PC2. Additionally, minimal or no expression of these transgenes is expected to be detectable by these methodologies in the samples from untreated kidneys.

### 16.10 Histological Evaluation

After euthanasia of pigs under anesthesia, treated and untreated pig kidneys are to be collected. Kidneys will be dissected covering all main sections including cortical, pyramidal, and papillary sections. Kidney tissues can be fixed in paraformaldehyde and can be embedded in paraffin or OCT. PC1 CTT or CTF can be detected using anti-CTT, anti-CTF, anti-HA antibodies, and PC2 protein can be detected using anti-PC2 or anti-HA antibodies for immunohistochemistry (IHC) of paraffin sections or immunofluorescence of OCT samples.

For IHC, paraffin sections can be subjected to Masson's trichrome staining and then incubated overnight at 4° C. with the recommended dilution of antibodies. Sections can be stained with Histofine Max PO and DAB chromogen after washing excess antibodies. After staining with a counterstain such as hematoxylin, slides can be visualized with microscopy.

For IF, other markers can be included to co-stain for specific type of cells as shown in Table 6 that will be visualized with fluorescence microscopy.

TABLE 6
Specific markers for IF studies

| Cell | Marker |
|---|---|
| Vascular endothelial cells | CD31+ |
| Mesangial cells | MEIS1/2/3+ |
| Podocytes | NPHS1+ or WT1+ |
| PT epithelial cells | LTL+ |
| Loop of Henle epithelial cells | UMOD+/SLC12A1+/LTL− |
| Macula densa | SLC12A1+/NNOS+ |
| DCT epithelial cells | UMOD+/SLC12A1−/ECAD+ |
| CD epithelial cells | ECAD+/GATA3+ |

It is contemplated that these methods will be able to detect PC1 CTT and CTF and PC2 in tubular epithelial cells of the treated kidneys, and an increase in total spotted protein of PC1 CTT and CTF and PC2 compared to the untreated group.

### 16.11 Evaluations of PKD1 AAV Constructs in PKD1 Pig Model of ADPKD

The purpose of this study is to evaluate PKD1 CTF and CTT-encoding constructs in a large animal model of ADPKD caused by mutations in PKD1 suitable for treatment with LRP-mediated delivery of AAV therapeutic constructs.

This study will utilize the PKD1 pig animal model (i.e., PKD1^{insG/+}) described by Watanabe et al., “Generation of heterozygous PKD1 mutant pigs exhibiting early-onset renal cyst formation,” Lab Invest. 2022, 102(5), 560-569, or other pig models of ADPKD should they become available.

The administered dose of PKD1 CTF or CTT-encoding constructs can be determined based on the above studies in healthy pigs. AAV therapeutic constructs can be administered to the left kidney as described in prior examples.

In-life and post-sacrifice assessments can be performed as described above. Additionally, specific assessments can be made periodically to monitor cyst growth in treated versus untreated pigs as follows.

Cyst formation can be imaged using ultrasound devices. Kidneys of pigs under anesthesia can be examined for cyst formation by a team of experts, which will include measurements of the kidney longitudinal and transverse diameters. Pigs will be followed up to 8-10 months of age. Further, post-sacrifice kidney weight and body weight % can be assessed.

Animals receiving therapeutically effective doses of AAV-delivered PKD1 CTF or CTT are advantageously expected to exhibit: (1) decreased serum and urine creatine; (2) decreased BUN; (3) increased urine exosomal PC1 CTF or CTT/TMEM2; (4) decreased kidney volume; and (5) decreased cyst size and decreased cyst number as compared to untreated PKD1 mutant pigs. Moreover, a decrease in interstitial fibrosis of the treated kidney is expected compared to the untreated diseased kidney.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A gene therapy vector adapted for transduction of renal cells of a human subject, the gene therapy vector comprising:
an adeno-associated virus (AAV) vector; and
a polynucleotide sequence packaged in the AAV vector, the polynucleotide sequence encoding a therapeutic protein having at least 80% sequence identity to a sequence selected from the group consisting of SEQ ID NO: 3, SEQ ID NO: 5, and SEQ ID NO: 7.

2. The gene therapy vector of claim 1, wherein the polynucleotide sequence encodes for a therapeutic protein having at least 80% sequence identity to SEQ ID NO: 3.

3. The gene therapy vector of claim 1, wherein the polynucleotide sequence encodes for a therapeutic protein having at least 80% sequence identity to SEQ ID NO: 5.

4. The gene therapy vector of claim 1, wherein the polynucleotide sequence encodes for a therapeutic protein having at least 80% sequence identity to SEQ ID NO: 7.

5. The gene therapy vector of claim 1, wherein the polynucleotide sequence further comprises a promoter sequence operatively linked to the polynucleotide sequence encoding for the therapeutic protein.

6. The gene therapy vector of claim 5, wherein the promoter sequence is selected from the group consisting of:
SEQ ID NO: 9,
SEQ ID NO: 10,
SEQ ID NO: 11,
SEQ ID NO: 12,
SEQ ID NO: 13, and
SEQ ID NO: 14.

7. The gene therapy vector of claim 5, wherein the promoter sequence is selected from the group consisting of:
SEQ ID NO: 17,
SEQ ID NO: 18,
SEQ ID NO: 19,
SEQ ID NO: 20,
SEQ ID NO: 21,
SEQ ID NO: 22,
SEQ ID NO: 23,
SEQ ID NO: 24,
SEQ ID NO: 25,
SEQ ID NO: 26,
SEQ ID NO: 27, and
SEQ ID NO: 28.

8. The gene therapy vector of claim 1, wherein a serotype of the AAV vector is selected from the group consisting of AAV1, AAV2, AAV3, AAV4, AAV5, AAV6, AAV7, AAV8, AAV9, AAV10, AAV11, AAV12, and AAV13.

9. The gene therapy vector of claim 1, wherein a serotype of the AAV vector is AAV5.

10. A gene therapy drug comprising:
the gene therapy vector of claim 1; and
a pharmaceutically acceptable carrier.

11. A method of treating a kidney-related disease comprising administering to a patient in need thereof a therapeutic dose of the gene therapy drug of claim 10.

12. A method of performing gene replacement of a mutated gene comprising administering to a patient in need thereof a therapeutic dose of the gene therapy drug of claim 10.

13. A method of treating autosomal dominant polycystic kidney disease (ADPKD) in a subject, the method comprising:
administering to the subject a therapeutic dose of a drug comprising the gene therapy vector of claim 1 and a pharmaceutically acceptable carrier.

14. A method of performing localized delivery of a polynucleotide sequence to renal cells in a kidney of a mammalian subject, the method comprising:
positioning a perfusion catheter in the renal artery of the kidney;
positioning a recovery catheter in the renal vein of the kidney, wherein the perfusion catheter and the recovery catheter together with a membrane oxygenation device form a closed perfusion circuit through the kidney; and
causing a perfusate to flow through the closed circuit, wherein the perfusate comprises the gene therapy drug of claim 10, and wherein the closed circuit substantially isolates perfusion through the kidney from the systemic circulation of the subject.

15. The method of claim 14, wherein the renal cells comprise tubular cells.

16. The method of claim 14, wherein a dose of the AAV vector is delivered via the closed circuit and maintained at a concentration of at least about 5×10⁹ of vector genome per milliliter (mL) of plasma) during perfusion, and wherein the vector present leaking into systemic circulation of the subject remains less than 5×10⁷ of vector genome per mL of plasma during perfusion, wherein the perfusion is maintained for a total of about 30 minutes to about 90 minutes.

17. The method of claim 14, wherein positioning the perfusion catheter in the renal artery comprises positioning the perfusion catheter via the arteria femoralis.

18. The method of claim 14, wherein positioning the recovery catheter in the renal vein comprises positioning the perfusion catheter via percutaneous access through the vena femoralis or via the jugular vein.

19-27. (canceled)

28. A method of delivering a therapeutic composition to a subject in need thereof, the method comprising locally delivering the therapeutic composition to a kidney of the subject while substantially avoiding introduction of the therapeutic composition into the systemic circulation or other organs, the therapeutic composition comprising the gene therapy drug of claim 10.


SEQUENCE LISTING (38 sequences)

SEQ ID NO: 1 - protein, 4303 aa, Homo sapiens
MPPAAPARLALALGLGLWLGALAGGPGRGCGPCEPPCLCGPAPGAACRVNCSGRGLRTLGPALRIPADATALDVSHNLLRALDVGLLANLSALAELDISNNKISTLEEGIFANLFNLSEINLSGNPFECDCGLAWLPRWAEEQQVRVVQPEAATCAGPGSLAGQPLLGIPLLDSGCGEEYVACLPDNSSGTVAAVSFSAAHEGLLQPEACSAFCFSTGQGLAALSEQGWCLCGAAQPSSASFACLSLCSGPPPPPAPTCRGPTLLQHVFPASPGATLVGPHGPLASGQLAAFHIAAPLPVTATRWDFGDGSAEVDAAGPAASHRYVLPGRYHVTAVLALGAGSALLGTDVQVEAAPAALELVCPSSVQSDESLDLSIQNRGGSGLEAAYSIVALGEEPARAVHPLCPSDTEIFPGNGHCYRLVVEKAAWLQAQEQCQAWAGAALAMVDSPAVQRFLVSRVTRSLDVWIGFSTVQGVEVGPAPQGEAFSLESCQNWLPGEPHPATAEHCVRLGPTGWCNTDLCSAPHSYVCELQPGGPVQDAENLLVGAPSGDLQGPLTPLAQQDGLSAPHEPVEVMVFPGLRLSREAFLTTAEFGTQELRRPAQLRLQVYRLLSTAGTPENGSEPESRSPDNRTQLAPACMPGGRWCPGANICLPLDASCHPQACANGCTSGPGLPGAPYALWREFLFSVPAGPPAQYSVTLHGQDVLMLPGDLVGLQHDAGPGALLHCSPAPGHPGPRAPYLSANASSWLPHLPAQLEGTWACPACALRLLAATEQLTVLLGLRPNPGLRLPGRYEVRAEVGNGVSRHNLSCSFDVVSPVAGLRVIYPAPRDGRLYVPTNGSALVLQVDSGANATATARWPGGSVSARFENVCPALVATFVPGCPWETNDTLFSVVALPWLSEGEHVVDVVVENSASRANLSLRVTAEEPICGLRATPSPEARVLQGVLVRYSPVVEAGSDMVFRWTINDKQSLTFQNVVFNVIYQSAAVFKLSLTASNHVSNVTVNYNVTVERMNRMQGLQVSTVPAVLSPNATLALTAGVLVDSAVEVAFLWTFGDGEQALHQFQPPYNESFPVPDPSVAQVLVEHNVMHTYAAPGEYLLTVLASNAFENLTQQVPVSVRASLPSVAVGVSDGVLVAGRPVTFYPHPLPSPGGVLYTWDFGDGSPVLTQSQPAANHTYASRGTYHVRLEVNNTVSGAAAQADVRVFEELRGLSVDMSLAVEQGAPVVVSAAVQTGDNITWTFDMGDGTVLSGPEATVEHVYLRAQNCTVTVGAASPAGHLARSLHVLVFVLEVLRVEPAACIPTQPDARLTAYVTGNPAHYLFDWTFGDGSSNTTVRGCPTVTHNFTRSGTFPLALVLSSRVNRAHYFTSICVEPEVGNVTLQPERQFVQLGDEAWLVACAWPPFPYRYTWDFGTEEAAPTRARGPEVTFIYRDPGSYLVTVTASNNISAANDSALVEVQEPVLVTSIKVNGSLGLELQQPYLFSAVGRGRPASYLWDLGDGGWLEGPEVTHAYNSTGDFTVRVAGWNEVSRSEAWLNVTVKRRVRGLVVNASRTVVPLNGSVSFSTSLEAGSDVRYSWVLCDRCTPIPGGPTISYTFRSVGTFNIIVTAENEVGSAQDSIFVYVLQLIEGLQVVGGGRYFPTNHTVQLQAVVRDGTNVSYSWTAWRDRGPALAGSGKGFSLTVLEAGTYHVQLRATNMLGSAWADCTMDFVEPVGWLMVAASPNPAAVNTSVTLSAELAGGSGVVYTWSLEEGLSWETSEPFTTHSFPTPGLHLVTMTAGNPLGSANATVEVDVQVPVSGLSIRASEPGGSFVAAGSSVPFWGQLATGTNVSWCWAVPGGSSKRGPHVTMVFPDAGTFSIRLNASNAVSWVSATYNLTAEEPIVGLVLWASSKVVAPGQLVHFQILLAAGSAVTFRLQVGGANPEVLPGPRFSHSFPRVGDHVVSVRGKNHVSWAQAQVRIVVLEAVSGLQVPNCCEPGIATGTERNFTARVQRGSRVAYAWYFSLQKVQGDSLVILSGRDVTYTPVAAGLLEIQVRAFNALGSENRTLVLEVQDAVQYVALQSGPCFTNRSAQFEAATSPSPRRVAYHWDFGDGSPGQDTDEPRAEHSYLRPGDYRVQVNASNLVSFFVAQATVTVQVLACREPEVDVVLPLQVLMRRSQRNYLEAHVDLRDCVTYQTEYRWEVYRTASCQRPGRPARVALPGVDVSRPRLVLPRLALPVGHYCFVFVVSFGDTPLTQSIQANVTVAPERLVPIIEGGSYRVWSDTRDLVLDGSESYDPNLEDGDQTPLSFHWACVASTQREAGGCALNFGPRGSSTVTIPRERLAAGVEYTFSLTVWKAGRKEEATNQTVLIRSGRVPIVSLECVSCKAQAVYEVSRSSYVYLEGRCLNCSSGSKRGRWAARTFSNKTLVLDETTTSTGSAGMRLVLRRGVLRDGEGYTFTLTVLGRSGEEEGCASIRLSPNRPPLGGSCRLFPLGAVHALTTKVHFECTGWHDAEDAGAPLVYALLLRRCRQGHCEEFCVYKGSLSSYGAVLPPGFRPHFEVGLAVVVQDQLGAAVVALNRSLAITLPEPNGSATGLTVWLHGLTASVLPGLLRQADPQHVIEYSLALVTVLNEYERALDVAAEPKHERQHRAQIRKNITETLVSLRVHTVDDIQQIAAALAQCMGPSRELVCRSCLKQTLHKLEAMMLILQAETTAGTVTPTAIGDSILNITGDLIHLASSDVRAPQPSELGAESPSRMVASQAYNLTSALMRILMRSRVLNEEPLTLAGEEIVAQGKRSDPRSLLCYGGAPGPGCHFSIPEAFSGALANLSDVVQLIFLVDSNPFPFGYISNYTVSTKVASMAFQTQAGAQIPIERLASERAITVKVPNNSDWAARGHRSSANSANSVVVQPQASVGAVVTLDSSNPAAGLHLQLNYTLLDGHYLSEEPEPYLAVYLHSEPRPNEHNCSASRRIRPESLQGADHRPYTFFISPGSRDPAGSYHLNLSSHFRWSALQVSVGLYTSLCQYFSEEDMVWRTEGLLPLEETSPRQAVCLTRHLTAFGASLFVPPSHVRFVFPEPTADVNYIVMLTCAVCLVTYMVMAAILHKLDQLDASRGRAIPFCGQRGRFKYEILVKTGWGRGSGTTAHVGIMLYGVDSRSGHRHLDGDRAFHRNSLDIFRIATPHSLGSVWKIRVWHDNKGLSPAWFLQHVIVRDLQTARSAFFLVNDWLSVETEANGGLVEKEVLAASDAALLRFRRLLVAELQRGFFDKHIWLSIWDRPPRSRFTRIQRATCCVLLICLFLGANAVWYGAVGDSAYSTGHVSRLSPLSVDTVAVGLVSSVVVYPVYLAILFLFRMSRSKVAGSPSPTPAGQQVLDIDSCLDSSVLDSSFLTFSGLHAEQAFVGQMKSDLFLDDSKSLVCWPSGEGTLSWPDLLSDPSIVGSNLRQLARGQAGHGLGPEEDGFSLASPYSPAKSFSASDEDLIQQVLAEGVSSPAPTQDTHMETDLLSSLSSTPGEKTETLALQRLGELGPPSPGLNWEQPQAARLSRTGLVEGLRKRLLPAWCASLAHGLSLLLVAVAVAVSGWVGASFPPGVSVAWLLSSSASFLASFLGWEPLKVLLEALYFSLVAKRLHPDEDDTLVESPAVTPVSARVPRVRPPHGFALFLAKEEARKVKRLHGMLRSLLVYMLFLLVTLLASYGDASCHGHAYRLQSAIKQELHSRAFLAITRSEELWPWMAHVLLPYVHGNQSSPELGPPRLRQVRLQEALYPDPPGPRVHTCSAAGGFSTSDYDVGWESPHNGSGTWAYSAPDLLGAWSWGSCAVYDSGGYVQELGLSLEESRDRLRFLQLHNWLDNRSRAVFLELTRYSPAVGLHAAVTLRLEFPAAGRALAALSVRPFALRRLSAGLSLPLLTSVCLLLFAVHFAVAEARTWHREGRWRVLRLGAWARWLLVALTAATALVRLAQLGAADRQWTRFVRGRPRRFTSFDQVAQLSSAARGLAASLLFLLLVKAAQQLRFVRQWSVFGKTLCRALPELLGVTLGLVVLGVAYAQLAILLVSSCVDSLWSVAQALLVLCPGTGLSTLCPAESWHLSPLLCVGLWALRLWGALRLGAVILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 2 - DNA, 12912 bp, Homo sapiens
  source 1..12912: mol_type = other DNA
atgccgcccgccgcgcccgcccgcctggcgctggccctgggcctgggcctgtggctcggggcgctggcggggggccccgggcgcggctgcgggccctgcgagcccccctgcctctgcggcccagcgcccggcgccgcctgccgcgtcaactgctcgggccgcgggctgcggacgctcggtcccgcgctgcgcatccccgcggacgccacagcgctagacgtctcccacaacctgctccgggcgctggacgttgggctcctggcgaacctctcggcgctggcagagctggatataagcaacaacaagatttctacgttagaagaaggaatatttgctaatttatttaatttaagtgaaataaacctgagtgggaacccgtttgagtgtgactgtggcctggcgtggctgccgcgatgggcggaggagcagcaggtgcgggtggtgcagcccgaggcagccacgtgtgctgggcctggctccctggctggccagcctctgcttggcatccccttgctggacagtggctgtggtgaggagtatgtcgcctgcctccctgacaacagctcaggcaccgtggcagcagtgtccttttcagctgcccacgaaggcctgcttcagccagaggcctgcagcgccttctgcttctccaccggccagggcctcgcagccctctcggagcagggctggtgcctgtgtggggcggcccagccctccagtgcctcctttgcctgcctgtccctctgctccggccccccgccacctcctgcccccacctgtaggggccccaccctcctccagcacgtcttccctgcctccccaggggccaccctggtggggccccacggacctctggcctctggccagctagcagccttccacatcgctgccccgctccctgtcactgccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagcctcgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccgggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggaggcagcttcgtggcggccgggtcctctgtgcccttttgggggcagctggccacgggcaccaatgtgagctggtgctgggctgtgcccggcggcagcagcaagcgtggccctcatgtcaccatggtcttcccggatgctggcaccttctccatccggctcaatgcctccaacgcagtcagctgggtctcagccacgtacaacctcacggcggaggagcccatcgtgggcctggtgctgtgggccagcagcaaggtggtggcgcccgggcagctggtccattttcagatcctgctggctgccggctcagctgtcaccttccgcctgcaggtcggcggggccaaccccgaggtgctccccgggccccgtttctcccacagcttcccccgcgtcggagaccacgtggtgagcgtgcggggcaaaaaccacgtgagctgggcccaggcgcaggtgcgcatcgtggtgctggaggccgtgagtgggctgcaggtgcccaactgctgcgagcctggcatcgccacgggcactgagaggaacttcacagcccgcgtgcagcgcggctctcgggtcgcctacgcctggtacttctcgctgcagaaggtccagggcgactcgctggtcatcctgtcgggccgcgacgtcacctacacgcccgtggccgcggggctgttggagatccaggtgcgcgccttcaacgccctgggcagtgagaaccgcacgctggtgctggaggttcaggacgccgtccagtatgtggccctgcagagcggcccctgcttcaccaaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtggcctcgatggcattccagacacaggccggcgcccagatccccatcgagcggctggcctcagagcgcgccatcaccgtgaaggtgcccaacaactcggactgggctgcccggggccaccgcagctccgccaactccgccaactccgttgtggtccagccccaggcctccgtcggtgctgtggtcaccctggacagcagcaaccctgcggccgggctgcatctgcagctcaactatacgctgctggacggccactacctgtctgaggaacctgagccctacctggcagtctacctacactcggagccccggcccaatgagcacaactgctcggctagcaggaggatccgcccagagtcactccagggtgctgaccaccggccctacaccttcttcatttccccggggagcagagacccagcggggagttaccatctgaacctctccagccacttccgctggtcggcgctgcaggtgtccgtgggcctgtacacgtccctgtgccagtacttcagcgaggaggacatggtgtggcggacagaggggctgctgcccctggaggagacctcgccccgccaggccgtctgcctcacccgccacctcaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgagcaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttag

SEQ ID NO: 3 - protein, 1256 aa, synthetic construct
MTAFGASLFVPPSHVRFVFPEPTADVNYIVMLTCAVCLVTYMVMAAILHKLDQLDASRGRAIPFCGQRGRFKYEILVKTGWGRGSGTTAHVGIMLYGVDSRSGHRHLDGDRAFHRNSLDIFRIATPHSLGSVWKIRVWHDNKGLSPAWFLQHVIVRDLQTARSAFFLVNDWLSVETEANGGLVEKEVLAASDAALLRFRRLLVAELQRGFFDKHIWLSIWDRPPRSRFTRIQRATCCVLLICLFLGANAVWYGAVGDSAYSTGHVSRLSPLSVDTVAVGLVSSVVVYPVYLAILFLFRMSRSKVAGSPSPTPAGQQVLDIDSCLDSSVLDSSFLTFSGLHAEQAFVGQMKSDLFLDDSKSLVCWPSGEGTLSWPDLLSDPSIVGSNLRQLARGQAGHGLGPEEDGFSLASPYSPAKSFSASDEDLIQQVLAEGVSSPAPTQDTHMETDLLSSLSSTPGEKTETLALQRLGELGPPSPGLNWEQPQAARLSRTGLVEGLRKRLLPAWCASLAHGLSLLLVAVAVAVSGWVGASFPPGVSVAWLLSSSASFLASFLGWEPLKVLLEALYFSLVAKRLHPDEDDTLVESPAVTPVSARVPRVRPPHGFALFLAKEEARKVKRLHGMLRSLLVYMLFLLVTLLASYGDASCHGHAYRLQSAIKQELHSRAFLAITRSEELWPWMAHVLLPYVHGNQSSPELGPPRLRQVRLQEALYPDPPGPRVHTCSAAGGFSTSDYDVGWESPHNGSGTWAYSAPDLLGAWSWGSCAVYDSGGYVQELGLSLEESRDRLRFLQLHNWLDNRSRAVFLELTRYSPAVGLHAAVTLRLEFPAAGRALAALSVRPFALRRLSAGLSLPLLTSVCLLLFAVHFAVAEARTWHREGRWRVLRLGAWARWLLVALTAATALVRLAQLGAADRQWTRFVRGRPRRFTSFDQVAQLSSAARGLAASLLFLLLVKAAQQLRFVRQWSVFGKTLCRALPELLGVTLGLVVLGVAYAQLAILLVSSCVDSLWSVAQALLVLCPGTGLSTLCPAESWHLSPLLCVGLWALRLWGALRLGAVILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 4 - DNA, 3771 bp, synthetic construct
  source 1..3771: mol_type = other DNA
atgaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgagcaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttag

SEQ ID NO: 5 - protein, 201 aa, synthetic construct
MVILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 6 - DNA, 606 bp, synthetic construct
  source 1..606: mol_type = other DNA
atggttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttag

SEQ ID NO: 7 - protein, 968 aa, Homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV

SEQ ID NO: 8 - DNA, 2907 bp, Homo sapiens
  source 1..2907: mol_type = other DNA
atggtgaactccagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatcttgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtgctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatga

SEQ ID NO: 9 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 10 - DNA, 1733 bp, synthetic construct
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 11 - DNA, 2589 bp, Homo sapiens
  source 1..2589: mol_type = genomic DNA
gccctcctatttagtctctctgccacctacaaattgagaaagtcaaatttagtaagtccttatagtttccagctctaaaacaacaggatttgtaacatcatgtagtgccaatcaattccagcctttccattaaataatccaaatgaatcagtattcatgcattcacttattcatatggatataatacttcaaaacactaactgggaataagtgtaatactttagaaagccttgcactctttcataatctttcaccttggaaacataaaagtaaacagctttaattgatcctccctttcttctagtaacccagttcccacagatgtcagtttcttaacaaatccaaacaaggagtaaagtattgagagtttcagaatgtgtgcatatcatattttcagcaaagggatgagcaacactttcaattaaaaaggaatctaaaagaaaacagcagcatccttgtaaaagtagatgaggacagccagctggacttagctgctaactcagcactcccttccccaactcctccaccttccagccatgctgcattgacctttttatcatggaatggtatgcagcttctctgattttgtcattgtcactcataatcccagcagacttgtgttataagcagctgcttatattgctgttatgtatagatatagaaatgtaacttgtcagtggtcaaagaagccaaggaaactgagcaaaggtgactgagaacaagtgctcagcatgggctccaacacccagtagggccttttctcattgatttttctcctacaaggaatttctctcttattgaaagacagtacagtttggggaatatcatttgaccagatatcctgctccattcttggaatgaaaaacaggtgcaattttagtataagagtctagctagggatgcggatgaaattaatttataacaggaccaggctgaggaaaaacaccaataaatgctacaaatagtttgtcactttttctaacagggaaatctctaaaggatgataaagatgctagctgtgtttaatagcaaatgttaatttgggggaatatgaaataatcatgatatctttctctctttgagccaaacgcagctttggaatttttctcactgtagtgttgtaggcagaaactatcaactgaggcaggcccttgcgaggaaatatttatcctccttagcccatgcaatgatcaggtttagacagtaaaaaatctatttaagtgactggattgctcatcttttatttactttactcccagtggcctaacctggaacatatagaagaaagtagtggaatggtttttccagacgcacttcagttacttcaggtcctcagtgtttaataaggttttgggagagaaaaaaagctattgctgtgtgaattcaataaatatttaaaaaatttaaaaaatcagtgagcattatttctgccatcatgtgatctgaactatctgagtagtatcataaatcttgggtgatgggttaccttgaccaacctccctttcagtaagaaacaaattattaacagaaagtgaaagagaaataaacctattcaatgtttcaagtccctcagaagagggtgaggcagctccccagttcgttgctagatccagcctggccaatgaaccctgaaaaagcccaactcctgctttcatcatggaagcacgggacaaagtgtctcttcctaaaaacagaagttagaccagaccccttcctgcctatgattcttcaagaagcattgcatcatcaacatcaggcataagcattaataaagaccctaaataataacagagatgaaacacatcgcaaagagagttttcttttatcccctttaaaatgtaaatactcccaggaggaatcagccaacatcattaggggttaatgcatatgtagaataactagggccaggatataaaataagaaatatgtagggaggagagaaaggcatccttgagacgactccaagaaggaaagttggggatgaggcgaaatttctgattttaccttaaagtgaccctaattcgatgaccttttgtggtttttttcttttttcttttttacttggccctgcccaagcaggacctaaaaacaaacagacaaaaaaggttactaacaactgttcctctccacgaaaatctgcagtaaaaggtaaaagatgtattcgttttgaagagaaaccagagcttgcgatgagcttctgtatctccgtcagccctctagcatgacattaggaaccctccaggagatgagtcttcacagcccgggttggcacctgcagacacgcacttttcaacgcccgcaccctgcccggggccggctctcccacccaggcctctctctgcttcagcgccgccccggccgtgggagtcggcgggcgcagtccacagctccaccaagacacagctgtcggggttccgggtgcgccccgcccgcggccccggtgtcccgcccctcgccctcagcccccacccgacggtctttagggtcccccgggcacgccacgcggacccgcagcgactccacagggactgcgctcccgtgcccctagcgctcccgcgctgctgctccagccgcccggcagctctgagg

SEQ ID NO: 12 - DNA, 265 bp, synthetic construct
  source 1..265: mol_type = other DNA
ggccctggggtcacggaggctggggaggcaccgaggaacgcgcctggcatgtgctgacagggaattttatgctccaggagcaagacagagagagacactcacagggaagaggggaagaggaaaacgagaaagggaggagagtaacggaaagagataaaaaagaaaagcaggtggcagagacacagagagagggacccagagaaagccagacagacgcaggtggctggcagcgggcgctgtgggggtcacagtagggggacctgtc

SEQ ID NO: 13 - DNA, 324 bp, synthetic construct
  source 1..324: mol_type = other DNA
aagtgggagccaagtctgaacacacacacacacacacacacacacacacacagaagttatgtctaagcccagagggccagcaagtgctcattgggctgtgtcaagggggcagtgacagaccaagagctgcccacctcctagggctggcagtcacggatgctgagcagatctggctctccaaagtcaataagtaacttggggggactaggcggggccaggcctgctcctgtgggccccggtggcattttccactcctgagcaagcacggccagaccgcctacctgctcaagtgtccaccttgcctcgccccacctaagccaaatt

SEQ ID NO: 14 - DNA, 1341 bp, synthetic construct
  source 1..1341: mol_type = other DNA
agcttgctctgccatgggaaggtccccaaacctgaaaaagaaacccaggcgccttgggcaaacggcttagcctctctgtaccccagaggggtatgacaaggggacagttccctcttcagaatgagtgggtgggatagcagcgatatgtgctcacagaagttctgggtgctggttagtaggcactcagtatgtgtgagacctccttcaacaaccccacatatagcactaacctaggctcagtgcctcagtgatcctaaaacagacactcagcttgtcccagtcccgcttccatctggaatgctgaccagatataccatgagcagtcctcgggggagaccccaaaacatggaaggaaagtgagaggagggagaccagggtcttcatactctgtaccctcatctgagattcccagaaaacaaggtttctgctgggtctgcctttcaggccttacagggggaggtgaggaggccctggtcagccctgtaactccctcaaagtcacctgagctggctcggtcttctcttttctgcttagagacatgagacaggtcagagcctcacccttatctgtgttcaggagaccatgtgaggctaggagagcttcagggggacctctaggcttctgtcccacccactggtctcactgatgaatgttctagagctccgaacacacacttggggtcagctgccctttgggttacactcccagtcctttccttagctccacgggccacttggggcaggacaaggtcatagcctactcagaggaactccgaagctaatacgtgagaaacaagctggtgtcttctgggcatccgtagaggcagttcatggtatggagtgggggagcctaatccagcctgtgaatgtaagggctttcctgaagaaggggttattagtgcttgttgctaaagcgtaagcaggtacttactgtcctgtagaaaggtgggaagagcattccaggcacaaggaacaatatctggagagatctggctacagatgccactgcacagaggagaagtgggagccaagtctgaacacacacacacacacacacacacacacacacagaagttatgtctaagcccagagggccagcaagtgctcattgggctgtgtcaagggggcagtgacagaccaagagctgcccacctcctagggctggcagtcacggatgctgagcagatctggctctccaaagtcaataagtaacttggggggactaggcggggccaggcctgctcctgtgggccccggtggcattttccactcctgagcaagcacggccagaccgcctacctgctcaagtgtccaccttgcctcgccccacctaagccaaatt

SEQ ID NO: 15 - DNA, 81 bp, Homo sapiens
  source 1..81: mol_type = genomic DNA
aggcatggtattttcagatcaagtgagggtccccaggccttcctggctctgtcacttattgactgtgtgaccctaggcaaa

SEQ ID NO: 16 - DNA, 191 bp, Homo sapiens
  source 1..191: mol_type = genomic DNA
aggcatggtattttcagatcaagtgagggtccccaggccttcctggctctgtcacttattgactgtgtgaccctaggcaaaccactcagcttctctgtgccccagcatgtgcccaatgccccttcagaacgcagtatgtggcataatgaaggtatgtgctcagagcagtgctgggcacagagcaggagctt

SEQ ID NO: 17 - DNA, 205 bp, Homo sapiens
  source 1..205: mol_type = genomic DNA
gtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagccc

SEQ ID NO: 18 - DNA, 241 bp, Homo sapiens
  source 1..241: mol_type = genomic DNA
gtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctctt

SEQ ID NO: 19 - DNA, 324 bp, Homo sapiens
  source 1..324: mol_type = genomic DNA
accaagtctggagccacagaaaaagtaacgtccagaaccagagggccaaggaatacctcctgggctgtgctggggtcgagggagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctctt

SEQ ID NO: 20 - DNA, 495 bp, Homo sapiens
  source 1..495: mol_type = genomic DNA
tgggaacataggggaagtttcctggaaaaaggaatatttgagctgagcactgaagcatgcatgagcaggtgcttaccacacagatgaatgagtgggaagggtgttccatgcaaaaggaacagagtgtgaaaaggtgtggctgcagacactggagcctgggagataagtcagaccaagtctggagccacagaaaaagtaacgtccagaaccagagggccaaggaatacctcctgggctgtgctggggtcgagggagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctctt

SEQ ID NO: 21 - DNA, 286 bp, synthetic construct
  source 1..286: mol_type = other DNA
aggcatggtattttcagatcaagtgagggtccccaggccttcctggctctgtcacttattgactgtgtgaccctaggcaaagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagccc

SEQ ID NO: 22 - DNA, 396 bp, synthetic construct
  source 1..396: mol_type = other DNA
aggcatggtattttcagatcaagtgagggtccccaggccttcctggctctgtcacttattgactgtgtgaccctaggcaaaccactcagcttctctgtgccccagcatgtgcccaatgccccttcagaacgcagtatgtggcataatgaaggtatgtgctcagagcagtgctgggcacagagcaggagcttgtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagccc

SEQ ID NO: 23 - DNA, 322 bp, synthetic construct
  source 1..322: mol_type = other DNA
aggcatggtattttcagatcaagtgagggtccccaggccttcctggctctgtcacttattgactgtgtgaccctaggcaaagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctctt

SEQ ID NO: 24 - DNA, 432 bp, synthetic construct
  source 1..432: mol_type = other DNA
aggcatggtattttcagatcaagtgagggtccccaggccttcctggctctgtcacttattgactgtgtgaccctaggcaaaccactcagcttctctgtgccccagcatgtgcccaatgccccttcagaacgcagtatgtggcataatgaaggtatgtgctcagagcagtgctgggcacagagcaggagcttgtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctctt

SEQ ID NO: 25 - DNA, 405 bp, synthetic construct
  source 1..405: mol_type = other DNA
aggcatggtattttcagatcaagtgagggtccccaggccttcctggctctgtcacttattgactgtgtgaccctaggcaaaaccaagtctggagccacagaaaaagtaacgtccagaaccagagggccaaggaatacctcctgggctgtgctggggtcgagggagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctctt

SEQ ID NO: 26 - DNA, 515 bp, synthetic construct
  source 1..515: mol_type = other DNA
aggcatggtattttcagatcaagtgagggtccccaggccttcctggctctgtcacttattgactgtgtgaccctaggcaaaccactcagcttctctgtgccccagcatgtgcccaatgccccttcagaacgcagtatgtggcataatgaaggtatgtgctcagagcagtgctgggcacagagcaggagcttaccaagtctggagccacagaaaaagtaacgtccagaaccagagggccaaggaatacctcctgggctgtgctggggtcgagggagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctctt

SEQ ID NO: 27 - DNA, 576 bp, synthetic construct
  source 1..576: mol_type = other DNA
aggcatggtattttcagatcaagtgagggtccccaggccttcctggctctgtcacttattgactgtgtgaccctaggcaaatgggaacataggggaagtttcctggaaaaaggaatatttgagctgagcactgaagcatgcatgagcaggtgcttaccacacagatgaatgagtgggaagggtgttccatgcaaaaggaacagagtgtgaaaaggtgtggctgcagacactggagcctgggagataagtcagaccaagtctggagccacagaaaaagtaacgtccagaaccagagggccaaggaatacctcctgggctgtgctggggtcgagggagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctctt

SEQ ID NO: 28 - DNA, 686 bp, synthetic construct
  source 1..686: mol_type = other DNA
aggcatggtattttcagatcaagtgagggtccccaggccttcctggctctgtcacttattgactgtgtgaccctaggcaaaccactcagcttctctgtgccccagcatgtgcccaatgccccttcagaacgcagtatgtggcataatgaaggtatgtgctcagagcagtgctgggcacagagcaggagctttgggaacataggggaagtttcctggaaaaaggaatatttgagctgagcactgaagcatgcatgagcaggtgcttaccacacagatgaatgagtgggaagggtgttccatgcaaaaggaacagagtgtgaaaaggtgtggctgcagacactggagcctgggagataagtcagaccaagtctggagccacagaaaaagtaacgtccagaaccagagggccaaggaatacctcctgggctgtgctggggtcgagggagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctctt

SEQ ID NO: 29 - DNA, 102 bp, synthetic construct
  source 1..102: mol_type = other DNA
ctgtgccttctagttgccagccatctgttgtttgcccctcccccgtgccttccttgaccctggaaggtgccactcccactgtcctttcctaataaaatgagg

SEQ ID NO: 30 - protein, 9 aa, synthetic construct
YPYDVPDYA

SEQ ID NO: 31 - DNA, 30 bp, synthetic construct
  source 1..30: mol_type = other DNA
atgtacccatacgatgttccagattacgct

SEQ ID NO: 32 - DNA, 5428 bp, synthetic construct
  source 1..5428: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagccccaagtttgtacaaaaaagcaggctgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 33 - DNA, 5464 bp, synthetic construct
  source 1..5464: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctcttcaagtttgtacaaaaaagcaggctgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 34 - DNA, 5545 bp, synthetic construct
  source 1..5545: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgaggcatggtattttcagatcaagtgagggtccccaggccttcctggctctgtcacttattgactgtgtgaccctaggcaaagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctcttcaagtttgtacaaaaaagcaggctgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 35 - DNA, 5655 bp, synthetic construct
  source 1..5655: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgaggcatggtattttcagatcaagtgagggtccccaggccttcctggctctgtcacttattgactgtgtgaccctaggcaaaccactcagcttctctgtgccccagcatgtgcccaatgccccttcagaacgcagtatgtggcataatgaaggtatgtgctcagagcagtgctgggcacagagcaggagcttgtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctcttcaagtttgtacaaaaaagcaggctgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtagacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 36 - DNA, 4812 bp, synthetic construct
  source 1..4812: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgaccaagtctggagccacagaaaaagtaacgtccagaaccagagggccaaggaatacctcctgggctgtgctggggtcgagggagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctcttcaagtttgtacaaaaaagcaggctgccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 37 - DNA, 4983 bp, synthetic construct
  source 1..4983: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtgggaacataggggaagtttcctggaaaaaggaatatttgagctgagcactgaagcatgcatgagcaggtgcttaccacacagatgaatgagtgggaagggtgttccatgcaaaaggaacagagtgtgaaaaggtgtggctgcagacactggagcctgggagataagtcagaccaagtctggagccacagaaaaagtaacgtccagaaccagagggccaaggaatacctcctgggctgtgctggggtcgagggagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctcttcaagtttgtacaaaaaagcaggctgccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 38 - DNA, 5174 bp, synthetic construct
  source 1..5174: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgaggcatggtattttcagatcaagtgagggtccccaggccttcctggctctgtcacttattgactgtgtgaccctaggcaaaccactcagcttctctgtgccccagcatgtgcccaatgccccttcagaacgcagtatgtggcataatgaaggtatgtgctcagagcagtgctgggcacagagcaggagctttgggaacataggggaagtttcctggaaaaaggaatatttgagctgagcactgaagcatgcatgagcaggtgcttaccacacagatgaatgagtgggaagggtgttccatgcaaaaggaacagagtgtgaaaaggtgtggctgcagacactggagcctgggagataagtcagaccaagtctggagccacagaaaaagtaacgtccagaaccagagggccaaggaatacctcctgggctgtgctggggtcgagggagtgccaggccaaccatctccccacctcctggagctggcagtcacggatgctgagcagctcctggctttccaaagtcaatgagtaacttggggggctgggcggggccaggcctgctgctgtgggcccagtggccttttccactcctgagcaagcgtggctgaaccctcctacctgctcaagtgccctgccttgccccacccagcccagcctggccagagccccctggagaaggagctctcttcaagtttgtacaaaaaagcaggctgccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaacccagctttcttgtacaaagtgggaattcgagcatcttaccgccatttatacccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag